(19) United States
(12) Patent Application Publication
(10) Pub. No.: US 2005/0032829 A1
(43) Pub. Date: Feb. 10, 2005

(54) Process for imidazo[4,5-c]pyridin-4-amines

(75) Inventors: Kyle J. Lindstrom, Houlton, WI (US); Luke T. Dressel, Somerset, WI (US); Jason D. Bonk, Hudson, WI (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/856,466

(22) Filed: May 28, 2004

Related U.S. Application Data
(60) Provisional application No. 60/476,662, filed on Jun. 6, 2003.

(51) Int. Cl. C07D 471/02; A61K 31/4745

(52) U.S. Cl. 514/303; 546/118

(57) ABSTRACT

A process and intermediates for preparing 1H-imidazo[4,5-c]pyridin-4-amines are disclosed. The process includes providing a 7H-imidazo[4,5-c]tetrazolo[1,5-a]pyridine and converting a 7H-imidazo[4,5-c]tetrazolo[1,5-a]pyridine to a 1H-imidazo[4,5-c]pyridin-4-amine.

### CROSS REFERENCE TO RELATED APPLICATIONS

This application claims the benefit of U.S. Provisional Application No. 60/476,662, filed Jun. 6, 2003.

### FIELD

This invention relates to processes for preparing 1H-imidazo[4,5-c]pyridin-4-amines and to intermediates for use in preparing 1H-imidazo[4,5-c]pyridin-4-amines.

### BACKGROUND

There has been a major effort in recent years to prepare and find compounds that modulate the immune system. Certain 1H-imidazopyridin-4-amine compounds useful as immune response modifiers and methods for their preparation are described, for example, in U.S. Pat. Nos. 5,446,153; 5,494,916; 5,644,063; 6,525,064; 6,545,016; and 6,545,017, International Publication No. WO 02/46194, and U.S. Patent Publication No. US 2004/0010007.

However, despite these developments, there is a continuing need for useful, alternative processes and intermediates for preparing immune response modifying imidazo[4,5-c]pyridin-4-amines.

### SUMMARY

It has now been found that 1H-imidazo[4,5-c]pyridin-4-amine compounds of the Formula I 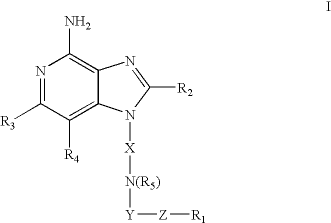
and pharmaceutically acceptable salts thereof, wherein
X is alkylene or alkenylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ is aryl, heteroaryl, heterocyclyl, alkyl or alkenyl, each of which may be unsubstituted or substituted by one or more substituents independently selected from:
   -alkyl;
   -alkenyl;
   -aryl;
   -heteroaryl;
   -heterocyclyl;
   -substituted cycloalkyl;
   -substituted aryl;
   -substituted heteroaryl;
   -substituted heterocyclyl;
   —O-alkyl;
   —O-(alkylene)₀₋₁-aryl;
   —O-(alkylene)₀₋₁-substituted aryl;
   —O-(alkylene)₀₋₁-heteroaryl;
   —O-(alkylene)₀₋₁-substituted heteroaryl;
   —O-(alkylene)₀₋₁-heterocyclyl;
   —O-(alkylene)₀₋₁-substituted heterocyclyl;
   —COOH;
   —CO—O-alkyl;
   —CO-alkyl;
   —S(O)₀₋₂-alkyl;
   —S(O)₀₋₂-(alkylene)₀₋₁-aryl;
   —S(O)₀₋₂-(alkylene)₀₋₁-substituted aryl;
   —S(O)₀₋₂-(alkylene)₀₋₁-heteroaryl;
   —S(O)₀₋₂-(alkylene)₀₋₁-substituted heteroaryl;
   —S(O)₀₋₂-(alkylene)₀₋₁-heterocyclyl;
   —S(O)₀₋₂-(alkylene)₀₋₁-substituted heterocyclyl;
   -(alkylene)₀₋₁-N(R₆)₂;
   -(alkylene)₀₋₁-NR₆—CO—O-alkyl;
   -(alkylene)₀₋₁-NR₆—CO-alkyl;
   -(alkylene)₀₋₁-NR₆—CO-aryl;
   -(alkylene)₀₋₁-NR₆—CO-substituted aryl;
   -(alkylene)₀₋₁-NR₆—CO-heteroaryl;
   -(alkylene)₀₋₁-NR₆—CO-substituted heteroaryl;
   —P(O)(O-alkyl)₂;
   —N₃;
   -halogen;
   -haloalkyl;
   -haloalkoxy;
   —CO-haloalkyl;
   —CO-haloalkoxy;
   —NO₂;
   —CN;
   —OH;
   —SH; and in the case of alkyl, alkenyl, and heterocyclyl, oxo;
R₂ is selected from:
   -hydrogen;
   -alkyl;
   -alkenyl;
   -aryl;
   -substituted aryl;
   -heteroaryl;
   -substituted heteroaryl;
   -alkylene-O-alkyl;
   -alkylene-S-alkyl;
   -alkylene-O-aryl;
   -alkylene-S-aryl;
   -alkylene-O-alkenyl;
   -alkylene-S-alkenyl; and
   -alkyl or alkenyl substituted by one or more substituents selected from:
      —OH;
      -halogen;
      —N (R₆)₂;
      —CO—N(R₆)₂;
      —CS—N(R₆)₂;
      —SO₂—N(R₆)₂;
      —NR₆—CO—C₁₋₁₀alkyl;
      —NR₆—CS—C₁₋₁₀alkyl;
      —NR₆—SO₂—C₁₋₁₀alkyl;
      —CO—C₁₋₁₀alkyl;
      —CO—O—C₁₋₁₀alkyl;
      —N₃;
      -aryl;
      -substituted aryl;
      -heteroaryl;
      -substituted heteroaryl;
      -heterocyclyl;
      -substituted heterocyclyl;
      —CO-aryl;
      —CO-(substituted aryl);
      —CO-heteroaryl; and
      —CO-(substituted heteroaryl);
R₃ and R₄ are independently selected from hydrogen, alkyl, alkenyl, halogen, alkoxy, amino, alkylamino, dialkylamino and alkylthio;
R₅ is H or C₁₋₁₀alkyl, or when R₅ is C₁₋₁₀alkyl, then R₅ can join with a carbon atom of X to form a ring having the structure 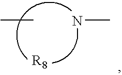
   or when R₅ is C₁₋₁₀alkyl, R₁ is alkyl, and Z is a bond, then R₅ and R₁ can join to form a ring having the structure 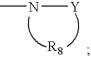
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 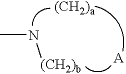
   wherein A is selected from —O—, —S(O)₀₋₂—, —N(R₆)—, and —CH₂—; and a and b are independently integers from 1 to 6 with the proviso that a+b is less than or equal to 7; and
R₈ is C₃₋₈alkylene;
   can be prepared by a process (I) comprising the steps of:
providing a compound of the Formula VIII 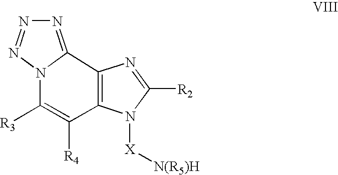
   wherein X, R₂, R₃, R₄, and R₅ are as defined above;
removing the tetrazolo ring from the compound of Formula VIII to provide a compound of Formula X 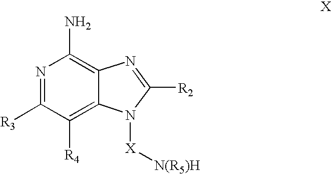
   wherein X, R₂, R₃, R₄, and R₅ are as defined above; and
reacting the compound of Formula X with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl, wherein R₁, R₇, and R₈ are as defined above, to provide a compound of Formula I; wherein the tetrazolo ring is removed from the compound of Formula VIII by (a) reductively removing the tetrazolo ring or (b) reacting the compound of Formula VIII with triphenylphosphine to provide an N-triphenylphosphinyl compound of Formula IX 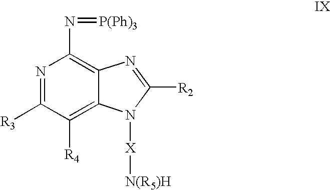
   wherein X, R₂, R₃, R₄, and R₅ are as defined above; and hydrolyzing the N-triphenylphosphinyl compound of Formula IX.

In some embodiments, the process (I) further comprises any one or more steps selected from steps (i), (ii), (iii), (iv), (v), and (vi):
(i) providing a compound of the Formula II 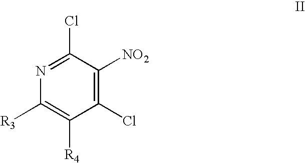
   wherein R₃ and R₄ are as defined above;
(ii) reacting the compound of Formula II(a) with an amine of the formula N(R₅)—X—NH₂ to provide a compound of Formula XIII 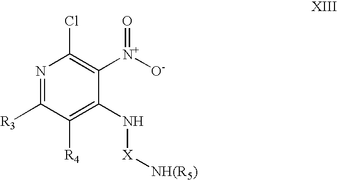
   wherein X, R₃, R₄, and R₅ are as defined above; and protecting the —N(R₅)— amino group with a protecting group B, or (b) with an amine of the formula B—N(R₅)—X—NH₂, to provide a compound of the Formula III 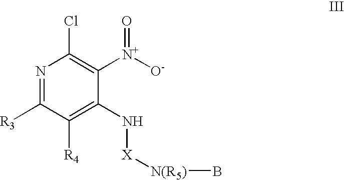
   wherein X, R₃, R₄, and R₅ are as defined above, and B is a protecting group for the —N(R₅)— amino group;
(iii) reacting the compound of Formula III with an alkali metal azide to provide a compound of Formula IV 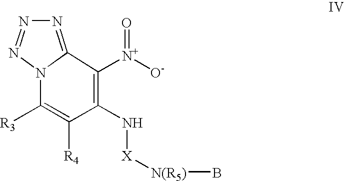
   wherein B, X, R₃, R₄, and R₅ are as defined above;
(iv) reducing the compound of Formula IV to provide a compound of the Formula V 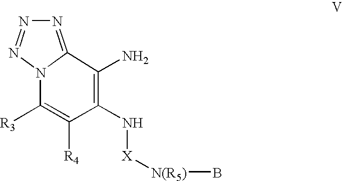
   wherein B, X, R₃, R₄, and R₅ are as defined above;
(v) reacting a compound of Formula V(a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein R₂ is as defined above and each alkyl contains 1 to 8 carbon atoms; to provide a compound of the Formula VII 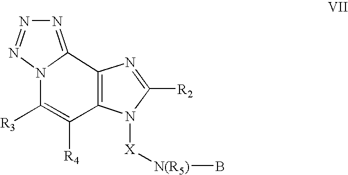
   wherein B, X, R₂, R₃, R₄, and R₅ are as defined above; and
(vi) removing the amine protecting group from the compound of Formula VII to provide a compound of the Formula VIII 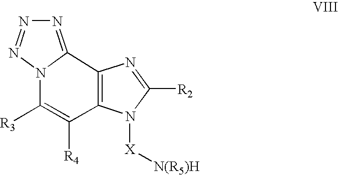
   wherein X, R₂, R₃, R₄, and R₅ are as defined above.

In another embodiment, 1H-imidazo[4,5-c]pyridin-4-amine compounds of the Formula I described above and pharmaceutically acceptable salts thereof, can be prepared by a process (II) comprising the steps of:
providing a compound of Formula XI 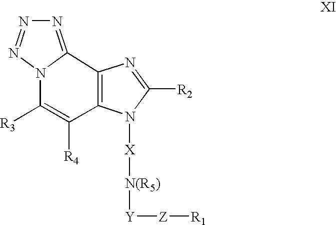
   wherein R₁, R₂, R₃, R₄, R₅, X, Y, and Z are as described above; and
removing the tetrazolo ring from the compound of Formula XI to provide a compound of Formula I; wherein the tetrazolo ring is removed from the compound of Formula XI by (a) reductively removing the tetrazolo ring or (b) reacting the compound of Formula XI with triphenylphosphine to provide an N-triphenylphosphinyl compound of Formula XII 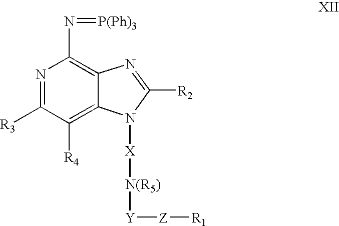
   wherein R₁, R₂, R₃, R₄, R₅, X, Y, and Z are as described above; and hydrolyzing the N-triphenylphosphinyl compound of Formula XII.

In some embodiments, the process (II) further comprises any one or more steps selected from steps (i), (ii), (iii), (iv), (v), and (vi) described above and (viii):
(viii) providing a compound of Formula VIII described above; and
reacting the compound of Formula VIII with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁-S(O)₂OS(O)₂—R₁, and R₁(R₇)N—S(O)₂Cl, wherein R₁, R₇, and R₈ are as defined above, to provide a compound of Formula XI 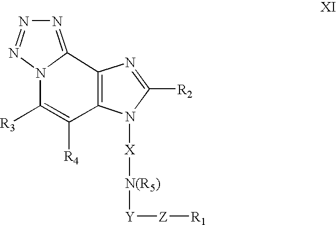
   wherein R₁, R₂, R₃, R₄, R₅, X, Y, and Z are as defined above.

In some embodiments, the process (II) further comprises any one or more steps selected from steps (i), (ii), and (iii) described above, and (x), (xi), (xii), and (xiii):
(x) providing a compound of Formula IV described above; and
removing the amine protecting group from the compound of Formula IV to provide a compound of the Formula XIV 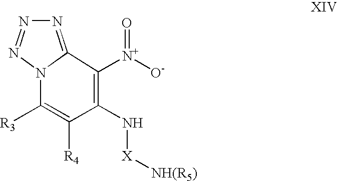
   wherein X, R₃, R₄, and R₅ are as defined above;
(xi) reacting the compound of Formula XIV with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl, wherein R₁, R₇, and R₈ are as defined above, to provide a compound of Formula XV 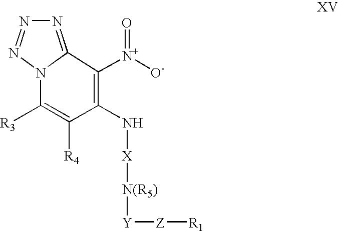
   wherein R₁, R₃, R₄, R₅, X, Y, and Z are as described above;
(xii) reducing the compound of Formula XV to provide a compound of the Formula XVI 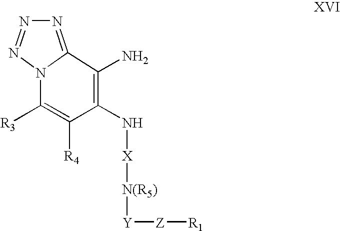
   wherein R₁, R₃, R₄, R₅, X, Y, and Z are as described above; and
(xiii) reacting a compound of Formula XVI (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein R₂ is as defined above and each alkyl contains 1 to 8 carbon atoms; to provide a compound of the Formula XI 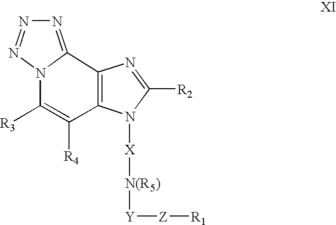
   wherein R₁, R₂, R₃, R₄, R₅, X, Y, and Z are as described above.

In some embodiments the above processes further comprise the step of isolating the compound of Formula I or a pharmaceutically acceptable salt thereof.

In another aspect this invention provides intermediates of the Formulas IV-VIII, XI, XIV, XV, and XVI, which are useful in the preparation of the compounds of Formula I, for example, in the processes described herein.

As used herein, “a,” “an,” “the,” “at least one,” and “one or more” are used interchangeably.

The terms “comprising” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. Guidance is also provided herein through lists of examples, which can be used in various combinations. In each instance, the recited list serves only as a representative group and should not be interpreted as an exclusive list.

### DETAILED DESCRIPTION OF ILLUSTRATIVE EMBODIMENTS OF THE INVENTION

Reaction Scheme I illustrates a process of the invention wherein R₁, R₂, R₃, R₄, R₅, X, Y and Z are as defined above.

In step (1) of Reaction Scheme I a 2,4-dichloro-3-nitropyridine of Formula II is reacted with an amine of formula B—N(R₅)—X—NH₂, wherein B is a protecting group for the —N(R₅)- amino group, examples of which include but are not limited to t-butoxycarbonyl, iso-butoxycarbonyl, benzyloxycarbonyl, ethoxycarbonyl, methoxycarbonyl, benzoyl, pivaloyl, propionyl, acetyl, and phthalimide; and R₅ and X are as defined above, to provide a 2-chloro-3-nitropyridine of Formula III. The reaction is preferably carried out by adding the amine to a solution of a compound of Formula II in a suitable solvent such as N,N-dimethylformamide in the presence of a tertiary amine such as triethylamine, and optionally heating. The product can be isolated from the reaction mixture using conventional methods.

Many 2,4-dichloro-3-nitropyridines of the Formula II are known and can be readily prepared using known synthetic methods. (See, for example, Dellaria et al, U.S. Pat. No. 6,525,064 and the references cited therein.)

Many amines of formula B—N(R₅)—X—NH₂ are known and others can be readily prepared using known synthetic methods. For example, a diamine, such as ethylenediamine, can be reacted with di-tert-butyl dicarbonate in a suitable solvent, such as ethyl acetate and the product isolated by conventional methods.

Alternatively, step 1 can be carried out by i) reacting a 2,4-dichloro-3-nitropyridine of Formula II with an amine of formula HN(R₅)—X—NH₂ and then ii) attaching the protecting group B to provide a 2-chloro-3-nitropyridine of Formula III. Step i) is preferably carried out by adding the amine to a solution of a compound of Formula II in a suitable solvent such as N,N-dimethylformamide in the presence of a tertiary amine such as triethylamine, and optionally heating. Step ii) is carried out using conventional methods for protecting an amine, for example, by reacting the amine intermediate with di-tert-butyl dicarbonate in a suitable solvent, such as tetrahydrofuran, in the presence of sodium hydroxide or reacting the amine intermediate with acetyl chloride in a suitable solvent, such as dichloromethane, in the presence of triethylamine.

In step (2) of Reaction Scheme I a 2-chloro-3-nitropyridine of Formula III is reacted with an alkali metal azide to provide an 8-nitrotetrazolo[1,5-a]pyridine of Formula IV. The reaction can be carried out by combining the compound of Formula III with an alkali metal azide, for example, sodium azide, in a suitable solvent such as anhydrous N,N-dimethylformamide, and heating to about 50-90° C., optionally in the presence of ammonium chloride. Alternatively, the reaction can be carried out by combining the compound of Formula III with an alkali metal azide, for example, sodium azide, in a suitable solvent such as 90/10 acetonitrile/H₂O in the presence of cerium III chloride, preferably cerium III chloride heptahydrate, optionally with heating, for example, at reflux. The product can be isolated from the reaction mixture using conventional methods.

In step (3) of Reaction Scheme I an 8-nitrotetrazolo[1,5-a]pyridine of Formula IV is reduced to provide a tetrazolo[1,5-a]pyridine-7,8-diamine of Formula V. The reduction can be carried out using a conventional heterogeneous hydrogenation catalyst, for example, platinum on carbon or palladium on carbon. The reaction can conveniently be carried out on a Parr apparatus in a suitable solvent such as ethanol, isopropanol, acetonitrile or toluene. Alternatively, Ni₂B can be generated in situ from sodium borohydride and NiCl₂ in the presence of methanol. The compound of Formula IV can be added to the reducing agent solution to effect reduction of the nitro group. When the compound of Formula IV contains an alkenyl or alkenylene moiety, the Ni₂B reducing agent can be used without reducing the alkenyl or alkenylene moiety. The product can be isolated from the reaction mixture using conventional methods.

In step (4) of Reaction Scheme I a tetrazolo[1,5-a]pyridine-7,8-diamine of Formula V is reacted with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof; or with an imidate of the formula alkyl-O—C(═N)—R₂; wherein R₂ is as defined above and each alkyl contains 1 to 8 carbon atoms, to provide a 7H-imidazo[4,5-c]tetrazolo[1,5-a]pyridine of Formula VII. When the carboxylic acid or equivalent thereof is used the reaction can be run in the absence of solvent or in an inert solvent such as, for example, toluene. The reaction may be run in the presence of cyclization conditions, which include sufficient heating (e.g., about 80-150° C.) to drive off any alcohol or water formed as a byproduct of the reaction, and optionally, in the presence of a catalyst such as pyridine hydrochloride. For example, an orthoester of the formula R₂C(O-alkyl)₃, (e.g., triethylorthoacetate) is combined with a tetrazolo[1,5-a]pyridine-7,8-diamine of Formula V in toluene in the presence of pyridine hydrochloride and heated at the reflux temperature. When the imidate is used the reaction can be run in a suitable solvent such as 1,2-dichloroethane at an elevated temperature, for example, about 60° C. The product can be isolated from the reaction mixture using conventional methods.

Alternatively, step 4 can include steps (4a) and (4b) of Reaction Scheme I. In step (4a) a tetrazolo[1,5-a]pyridine-7,8-diamine of Formula V is reacted with a carboxylic acid of the formula R₂CO₂H, the corresponding acyl halide, or a mixture thereof, wherein R₂ is as defined above, to provide an N-[tetrazolo[1,5-a]pyridin-8-yl]amide of Formula VI. The reaction can be run in an inert solvent such as toluene, dichloromethane, acetonitrile, or pyridine at a reduced temperature, for example about 0° C. For example, an acyl halide can be added to a solution of the compound of Formula V in dichloromethane at about 0° C. in the presence of triethylamine. The product can be isolated from the reaction mixture using conventional methods.

In step (4b) of Reaction Scheme I an N-[tetrazolo[1,5-a]pyridin-8-yl]amide of Formula VI is cyclized to provide a 7H-imidazo[4,5-c]tetrazolo[1,5-a]pyridine of Formula VII. The reaction can be run at an elevated temperature, such as a reflux temperature, sufficient to drive off any water formed as a by-product of the reaction. Optionally a catalyst such as pyridine hydrochloride can be included. The reaction can be run in the absence of a solvent or in an inert solvent, for example, a solvent having a boiling point of about 80° C. to about 150° C., preferably at least about 100° C., (e.g., toluene, pyridine). Alternatively, the reaction can be run in a mixture of water and a lower alkanol such as ethanol in the presence of a base such as sodium hydroxide. The product can be isolated from the reaction mixture using conventional methods.

In step (5) of Reaction Scheme I the amine protecting group of a 7H-imidazo[4,5-c]tetrazolo[1,5-a]pyridine of Formula VII is removed to provide a 7H-imidazo[4,5-c]tetrazolo[1,5-a]pyridine of Formula VIII. The reaction can be run by treating a solution of a 7H-imidazo[4,5-c]tetrazolo[1,5-a]pyridine of Formula VII in a suitable solvent such as, for example, dichloromethane or ethanol with an acid, for example, hydrochloric acid or trifluoroacetic acid, optionally with heating. The product can be isolated from the reaction mixture using conventional methods.

In step (6) of Reaction Scheme I, the tetrazolo ring is reductively removed from a 7H-imidazo[4,5-c]tetrazolo[1,5-a]pyridine of Formula VIII to provide a 1H-imidazo[4,5-c]pyridin-4-amine of Formula X. The reaction can be carried out by reacting the 7H-imidazo[4,5-c]tetrazolo[1,5-a]pyridine of Formula VIII with hydrogen in the presence of a catalyst and an acid. The reaction can be conveniently run in a Parr apparatus with a suitable catalyst, such as platinum IV oxide, and a suitable acid, such as trifluoroacetic acid or concentrated hydrochloric acid. The product can be isolated from the reaction mixture using conventional methods.

Alternatively, steps (6a) and (6b) can be used in place of step (6). In step (6a) of Reaction Scheme I a 7H-imidazo[4,5-c]tetrazolo[1,5-a]pyridine of Formula VIII is reacted with triphenylphosphine to form an N-triphenylphosphinyl compound of Formula IX. The reaction with triphenylphosphine can be run in a suitable solvent such as toluene or 1,2-dichlorobenzene under an atmosphere of nitrogen with heating, for example at the reflux temperature.

In step (6b) of Reaction Scheme I an N-triphenylphosphinyl compound of Formula IX is hydrolyzed to provide a 1H-imidazo[4,5-c]pyridin-4-amine of Formula X. The hydrolysis can be carried out by general methods well known to those skilled in the art, for example, by heating in a lower alkanol in the presence of an acid such as hydrochloric acid. The product can be isolated from the reaction mixture using conventional methods as the compound of Formula X or as a pharmaceutically acceptable salt thereof.

The use of (6a) and (6b) is preferred when protection of a 7H-imidazo[4,5-c]tetrazolo[1,5-a]pyridine of Formula VIII from the reductive conditions of step (6) is desired. For example, readily reducible moieties such as alkenyl and heteroaryl groups can be protected from reduction by using steps (6a) and (6b) in place of step (6).

In step (7) of Reaction Scheme I a 1H-imidazo[4,5-c]pyridin-4-amine of Formula X is reacted with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl to provide a compound of Formula I. For example, an acid chloride of formula R₁—C(O)Cl or R₁(R₇)N—C(O)Cl, or an acid anhydride of formula R₁—C(O)OC(O)—R₁ or R₁(R₇)N—C(O)OC(O)—N(R₇)R₁ is reacted with a 1H-imidazo[4,5-c]pyridin-4-amine of Formula X to provide a 1H-imidazo[4,5-c]pyridin-1-yl amide or urea, each being a subgenus of Formula I wherein Y is —CO— and Z is a bond or —N(R₇)—. The reaction is preferably carried out by adding the acid chloride or acid anhydride to a solution of a 1H-imidazo[4,5-c]pyridin-4-amine of Formula X in a suitable solvent such as dichloromethane or acetonitrile in the presence of a base such as triethylamine. The reaction can be run at a reduced temperature (for example, 0° C.) or at ambient temperature. The product or a pharmaceutically acceptable salt thereof can be isolated using conventional methods.

In another example, a 1H-imidazo[4,5-c]pyridin-4-amine of Formula X is reacted with an isocyanate of formula R₁—N═C═O, R₁—C(O)—N═C═O, or R₁—S(O)₂—N═C═O, or with an isothiocyanate of formula R₁—N═C═S, R₁—C(O)—N═C═S, or R₁—S(O)₂—N═C═S, to provide a 1H-imidazo[4,5-c]pyridin-1-yl urea or thiourea, each being a subgenus of Formula I wherein Y is —CO— or —CS—, and Z is —(NH)—, —NH—C(O)—, or —NH—S(O)₂—. The reaction is preferably carried out by adding the isocyanate or isothiocyanate to a solution of a compound of Formula X in a suitable solvent such as dichloromethane at a reduced temperature (for example, 0° C.). The product or a pharmaceutically acceptable salt thereof can be isolated using conventional methods.

In another example, a 1H-imidazo[4,5-c]pyridin-4-amine of Formula X is reacted with a sulfonyl chloride of formula R₁—S(O)₂Cl or R₁(R₇)N—S(O)₂Cl, or a sulfonic anhydride of formula R₁—S(O)₂OS(O)₂—R, to provide a 1H-imidazo[4,5-c]pyridin-1-yl sulfonamide or sulfamide, each being a subgenus of Formula I wherein Y is —SO₂—, and Z is a bond or —N(R₇)—. The reaction is preferably carried out by adding the sulfonyl chloride or sulfonic anhydride to a solution of a compound of Formula X in a suitable solvent such as dichloromethane in the presence of a base such as triethylamine.

In another example, a 1H-imidazo[4,5-c]pyridin-4-amine of Formula X is reacted with a chloroalkanoyl chloride or chloroalkanesulfonyl chloride compound of formula Cl—R₈—C(O)—Cl or Cl—R₈—S(O)₂Cl to provide a subgenus of compounds of Formula I wherein R₅ and R₁ join to form a ring having the structure 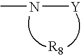
wherein Y is —C(O)— or —S(O)₂—. The reaction is preferably carried out by adding the chloroalkanoyl chloride or chloroalkanesulfonyl chloride compound to a solution of a compound of Formula X in a suitable solvent such as dichloromethane in the presence of a base such as triethylamine.

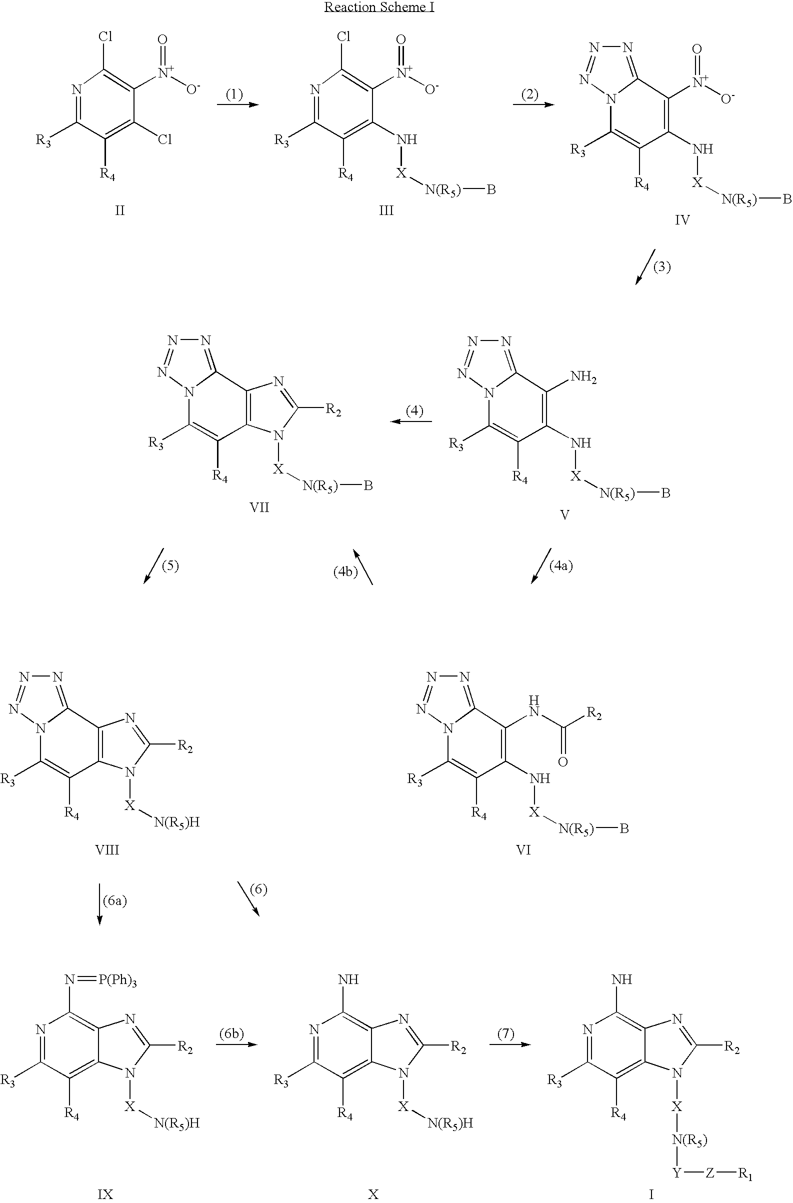

Reaction Scheme II illustrates another process of the invention wherein R₁, R₂, R₃, R₄, R₅, X, Y and Z are as defined above.

In step (1) of Reaction Scheme II a 7H-imidazo[4,5-c]tetrazolo[1,5-a]pyridine of Formula VIII is reacted with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl to provide a 7H-imidazo[4,5-c]tetrazolo[1,5-a]pyridine of Formula XI. This reaction can be carried out in the same manner as step (7) of Reaction Scheme I. The product can be isolated from the reaction mixture using conventional methods. The 7H-imidazo[4,5-c]tetrazolo[1,5-a]pyridine of Formula VIII may be provided using steps (1)-(6) of Reaction Scheme I.

In step (2) of Reaction Scheme II, the tetrazolo ring is reductively removed from a 7H-imidazo[4,5-c]tetrazolo[1,5-a]pyridine of Formula XI to provide a 1H-imidazo[4,5-c]pyridin-4-amine of Formula I or a pharmaceutically acceptable salt thereof. The reaction can be carried out by reacting the 7H-imidazo[4,5-c]tetrazolo[1,5-a]pyridine of Formula XI with hydrogen in the presence of a catalyst and an acid. The reaction can be conveniently run in a Parr apparatus with a suitable catalyst, such as platinum IV oxide, and a suitable acid, such as trifluoroacetic acid or concentrated hydrochloric acid. The product can be isolated from the reaction mixture using conventional methods.

Alternatively, steps (2a) and (2b) in Reaction Scheme II can be used in place of step (2) in Reaction Scheme II. In step (2a) of Reaction Scheme II a 7H-imidazo[4,5-c]tetrazolo[1,5-a]pyridine of Formula XI is reacted with triphenylphosphine to form an N-triphenylphosphinyl compound of Formula XII. The reaction with triphenylphosphine can be run in a suitable solvent such as toluene or 1,2-dichlorobenzene under an atmosphere of nitrogen with heating, for example at the reflux temperature.

In step (2b) of Reaction Scheme II an N-triphenylphosphinyl compound of Formula XII is hydrolyzed to provide a 1H-imidazo[4,5-c]pyridin-4-amine of Formula I. The hydrolysis can be carried out by general methods well known to those skilled in the art, for example, by heating in a lower alkanol in the presence of an acid such as hydrochloric acid. The product can be isolated from the reaction mixture using conventional methods as the compound of Formula I or as a pharmaceutically acceptable salt thereof.

The use of steps (2a) and (2b) is preferred when protection of a 7H-imidazo[4,5-c]tetrazolo[1,5-a]pyridine of Formula XI from the reductive conditions of step (2) is desired. For example, readily reducible moieties such as alkenyl and heteroaryl groups can be protected from reduction by using steps (2a) and (2b) in place of step (2).

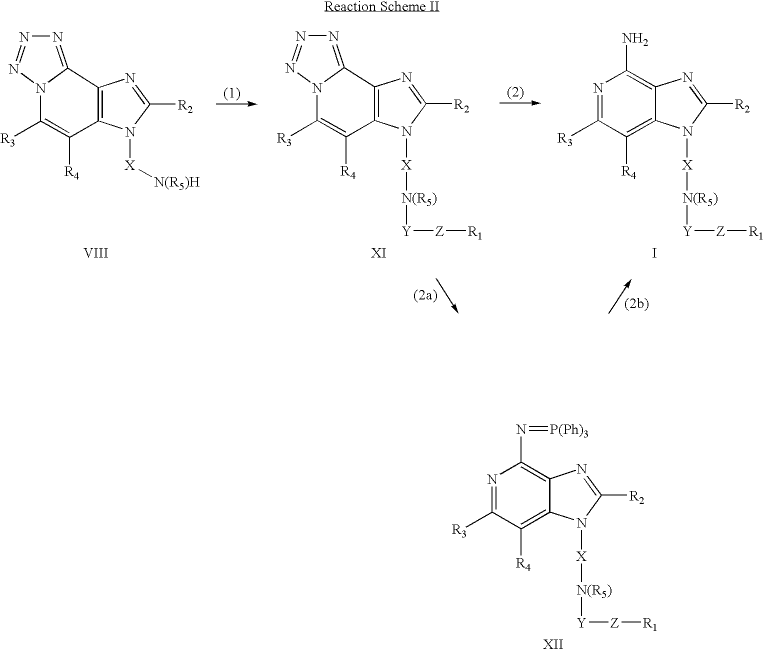

Reaction Scheme III illustrates a process of the invention wherein R₁, R₂, R₃, R₄, R₅, B, X, Y and Z are as defined above.

In step (1) of Reaction Scheme III the amine protecting group of an 8-nitrotetrazolo[1,5-a]pyridine of Formula IV is removed to provide an 8-nitrotetrazolo[1,5-a]pyridine of Formula XIV. The reaction can be carried out as in step (5) of Reaction Scheme I. The product can be isolated from the reaction mixture using conventional methods.

In step (2) of Reaction Scheme III an 8-nitrotetrazolo[1,5-a]pyridine of Formula XIV is reacted with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl to provide an 8-nitrotetrazolo[1,5-a]pyridine of Formula XV. The reaction can be carried out as in step (7) of Reaction Scheme I. The product can be isolated from the reaction mixture using conventional methods.

In step (3) of Reaction Scheme III an 8-nitrotetrazolo[1,5-a]pyridine of Formula XV is reduced to provide a tetrazolo[1,5-a]pyridine-7,8-diamine of Formula XVI. The reduction can be carried as in step (3) of Reaction Scheme I. The product can be isolated from the reaction mixture using conventional methods.

In step (4) of Reaction Scheme III a tetrazolo[1,5-a]pyridine-7,8-diamine of Formula XVI is reacted with an imidate of the formula alkyl-O—C(═N)—R₂ or with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, wherein R₂ is as defined above and each alkyl contains 1 to 8 carbon atoms to provide a 7H-imidazo[4,5-c]tetrazolo[1,5-a]pyridine of Formula XI. The reaction can be carried out as in step (4) or steps (4a) and (4b) of Reaction Scheme I. The product can be isolated from the reaction mixture using conventional methods.

In step (5) of Reaction Scheme III the tetrazolo ring is removed from a 7H-imidazo[4,5-c]tetrazolo[1,5-a]pyridine of Formula XI to provide a 1H-imidazo[4,5-c]pyridin-4-amine of Formula I. The reaction can be carried as in step (2) or steps (2a) and (2b) of Reaction Scheme II. The product can be isolated from the reaction mixture using conventional methods.

The use of steps (2a) and (2b) of Reaction Scheme II in step (5) of Reaction Scheme III is preferred when protection of a 7H-imidazo[4,5-c]tetrazolo[1,5-a]pyridine of Formula XI from the reductive conditions of step (2) of Reaction Scheme II is desired. For example, readily reducible moieties such as alkenyl and heteroaryl groups can be protected from reduction by using steps (2a) and (2b) in place of step (2) of Reaction Scheme II.

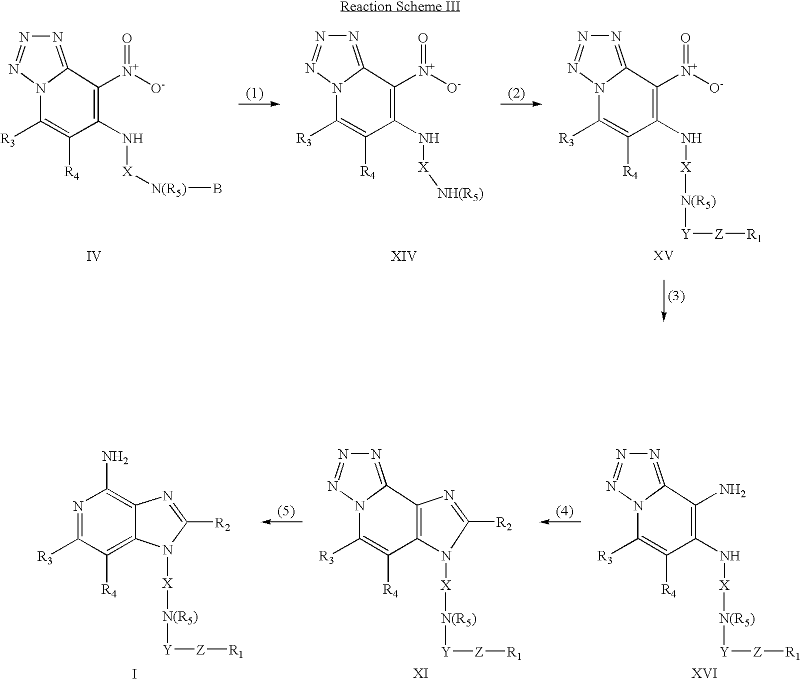

In one embodiment, the present invention provides a process (I-a) for preparing 1H-imidazo[4,5-c]pyridin-4-amine compounds of the Formula Ia 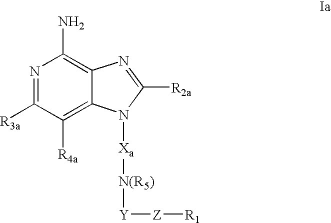
and pharmaceutically acceptable salts thereof, wherein Y, Z, R₁, and R₅ are as defined above; Xₐ is alkylene; R₂a is selected from:
-hydrogen,
-alkyl,
-alkylene-O-alkyl,
-alkylene-S-alkyl, and
-alkyl substituted by one or more substituents selected from:
   -OH,
   -halogen,
   —N(R₆)₂,
   —CO—N(R₆)₂,
   —CS—N(R₆)₂,
   —SO₂—N(R₆)₂,
   —NR₆—CO—C₁₋₁₀alkyl,
   —NR₆—CS—C₁₋₁₀alkyl,
   —NR₆—SO₂—C₁₋₁₀alkyl,
   —CO—C₁₋₁₀alkyl,
   —CO—O—C₁₋₁₀alkyl,
   —N₃,
   -heterocyclyl, and
   -substituted heterocyclyl; and
      R₃ₐ and R₄ₐ are independently selected from hydrogen, alkyl, halogen, alkoxy, amino, alkylamino, dialkylamino, and alkylthio; which process comprises the steps of:
providing a compound of the Formula VIIIa 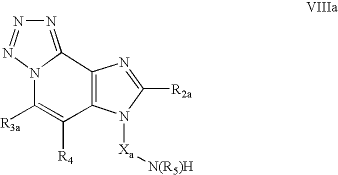
   wherein R₂ₐ, R₃ₐ, R₄ₐ, R₅, and Xₐ are as defined above;
reductively removing the tetrazolo ring from the compound of Formula VIIa to provide a compound of Formula Xa 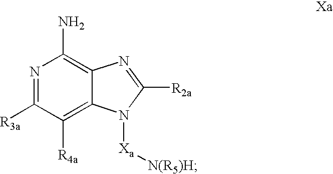
   and
reacting the compound of Formula Xa with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl, wherein R₁, R₇, and R₈ are as defined above; to provide a compound of the Formula Ia or a pharmaceutically acceptable salt thereof.

In another embodiment, the process (I-a) further comprises the step of isolating the compound of Formula Ia or a pharmaceutically acceptable salt thereof.

In some embodiments, a process (I-a-1) comprises the process (I-a) further comprising one or more steps selected from steps (i), (ii), (iii), (iv), (v), and (vi) described above wherein R₂, R₃, R₄, and X are R₂ₐ, R₃ₐ, R₄ₐ, and Xₐ, respectively, defined in process (I-a).

In one embodiment, the present invention provides a process (I-b) for preparing 1H-imidazo[4,5-c]pyridin-4-amine compounds of the Formula I and pharmaceutically acceptable salts thereof as defined above, which process comprises the steps of:
providing a compound of Formula VIII 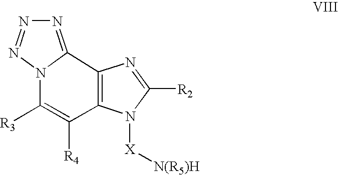
   wherein R₂, R₃, R₄, R₅, and X are as defined above;
reacting the compound of the Formula VIII with triphenylphosphine to provide an N-triphenylphosphinyl compound of Formula IX 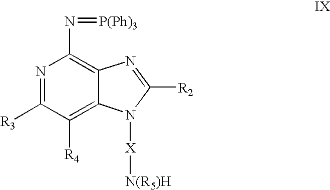
   wherein R₂, R₃, R₄, R₅, and X are as defined above;
hydrolyzing the N-triphenylphosphinyl compound of the Formula IX to provide a compound of Formula X 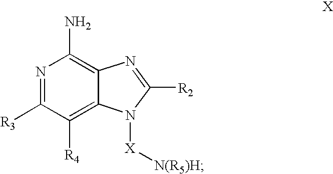
   and
reacting the compound of the Formula X with a compound selected from R₁—C(O)Cl, R₁(R₇)N—C(O)Cl, Cl—R₈—C(O)Cl, R—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl, wherein R₁, R₇, and R₈ are as defined above; to provide a compound of the Formula I or a pharmaceutically acceptable salt thereof as defined above.

In another embodiment, the process (I-b) further comprises the step of isolating the compound of Formula I or a pharmaceutically acceptable salt thereof.

In some embodiments, a process (I-b-1) comprises the process (I-b) further comprising one or more steps selected from (i), (ii), (iii), (iv), (v), and (vi) described above.

In one embodiment, the present invention provides a process (II-a) for preparing 1H-imidazo[4,5-c]pyridin-4-amine compounds of the Formula Ib 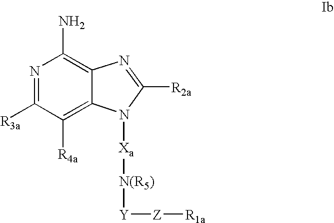
and pharmnaceutically acceptable salts thereof, wherein Xₐ, Y, Z, R₂ₐ, R₃ₐ, R₄ₐ, and R₅ are as defined above, and R₁ₐ is alkyl or heterocyclyl, each of which may be unsubstituted or substituted by one or more substituents independently selected from:
-alkyl;
-heterocyclyl;
-substituted cycloalkyl;
-substituted heterocyclyl;
—O-alkyl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₂-heterocyclyl;
—S(O)₀₋₂-(alkylene)₀₋₂-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl and heterocyclyl, oxo;
   which process comprises the steps of:
providing a compound of the Formula XIa 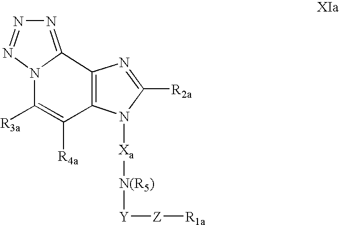
   wherein R₁ₐ, R₂ₐ, R₃ₐ, R₄ₐ, R₅, Xₐ, Y, and Z are as defined above; and
reductively removing the tetrazolo ring from the compound of Formula XIa to provide a compound of Formula Ib or a pharmaceutically acceptable salt thereof.

In another embodiment, the process (II-a) further comprises the step of isolating the compound of Formula Ib or a pharmaceutically acceptable salt thereof.

In another embodiment, a process (II-a-1) comprises the process (II-a) further comprising the steps of:
providing a compound of the Formula VIIIa 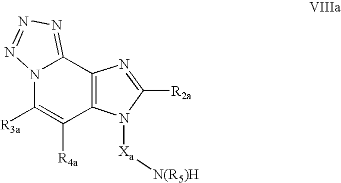
   wherein R₂ₐ, R₃ₐ, R₄ₐ, R₅, and Xₐ are as defined above; and
reacting the compound of Formula VIIIa with a compound selected from R₁ₐ—C(O)Cl, R₁ₐ(R₇)N—C(O)Cl, Cl—R₈—C(O)Cl, R₁ₐ—C(O)OC(O)—R₁ₐ, R₁ₐ(R₇)N—C(O)OC(O)—N(R₇)R₁ₐ, R₁ₐ—N═C═O, R₁ₐ—C(O)—N═C═O, R₁ₐ—S(O)₂—N═C═O, R₁ₐ—N═C═S, R₁ₐ—C(O)—N═C═S, R₁ₐ—S(O)₂—N═C═S, R_{1a—S(O)}₂Cl, Cl—R₈—S(O)₂Cl, R₁ₐ—S(O)₂OS(O)₂—R₁ₐ, and R₁ₐ(R₇)N—S(O)₂Cl, wherein R₁ₐ, R₇, and R₈ are as defined above, to provide a compound of Formula XIa defined above.

In some embodiments, a process (II-a-2) comprises the process (II-a-1) further comprising one or more steps selected from steps (i), (ii), (iii), (iv), (v), and (vi) described above wherein R₂, R₃, R₄, and X are R₂ₐ, R₃ₐ, R₄ₐ and Xₐ, respectively, defined in process (II-a-1).

In some embodiments, a process (II-a-3) comprises the process (II-a-1) further comprising one or more steps selected from steps (i), (ii), (iii), (x), (xi), (xii), and (xiii) described above wherein R₂, R₃, R₄, and X are R₂ₐ, R₃ₐ, R₄ₐ, and Xₐ, respectively, defined in process (II-a-1).

In one embodiment, the present invention provides a process (II-b) for preparing 1H-imidazo[4,5-c]pyridin-4-amine compounds of the Formula I and pharmaceutically acceptable salts thereof as defined above, which process comprises the steps of:
providing a compound of Formula XI 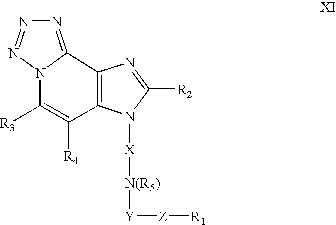
   wherein R₁, R₂, R₃, R₄, R₅, X, Y, and Z are as defined above;
reacting the compound of the Formula XI with triphenylphosphine to provide an N-triphenylphosphinyl compound of Formula XII 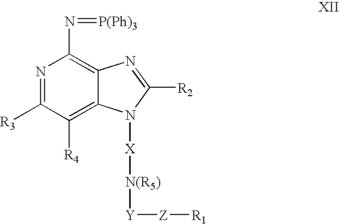
   wherein R₁, R₂, R₃, R₄, R₅, X, Y, and Z are as defined above; and
hydrolyzing the N-triphenylphosphinyl compound of the Formula XII to provide a compound of Formula I or a pharmaceutically acceptable salt thereof.

In another embodiment, the process (II-b) further comprises the step of isolating the compound of the Formula I or a pharmaceutically acceptable salt thereof.

In another embodiment, a process (I-b-1) comprises the process (II-b) further comprising the steps of:
providing a compound of the Formula VIII 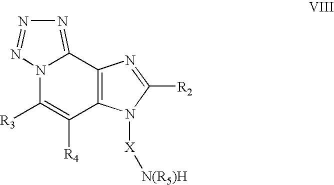
   wherein R₂, R₃, R₄, R₅, and X are as defined above; and
reacting the compound of Formula VIII with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)-N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl, wherein R₁, R₇, and R₈ are as defined above, to provide a compound of Formula XI defined above.

In some embodiments, a process (II-b-2) comprises the process (I-b-1) further comprising one or more steps selected from steps (i), (ii), (iii), (iv), (v), and (vi) described above.

In some embodiments, a process (II-b-3) comprises the process (I-b-1) further comprising one or more steps selected from steps (i), (ii), (iii), (x), (xi), (xii), and (xiii) described above.

In one embodiment, the present invention provides a process (III) for preparing a chemical compound comprising the steps of:
providing a compound of Formula III 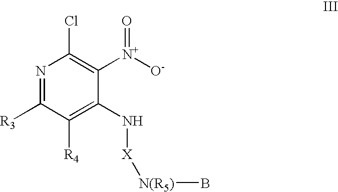
   wherein B, X, R₃, R₄, and R₅ are as defined above; and
reacting the compound of the Formula III with an alkali metal azide to provide a compound of Formula IV 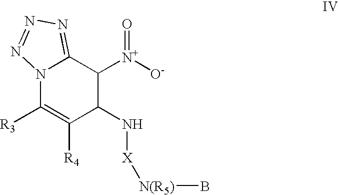
   wherein B, X, R₃, R₄, and R₅ are as defined above. In certain embodiments, the compound of Formula III is reacted with the alkali metal azide in the presence of cerium III chloride.

In another embodiment, the process (III) further comprises the step of isolating the compound of Formula IV.

In some embodiments, a process (III-a) comprises the process (III) further comprising one or more steps selected from steps (iv), (v), and (vi) described above and (vii-1) and (viii-a):
(vii-1) reductively removing the tetrazolo ring from a compound of the Formula VIIIa defined above to provide a compound of Formula Xa 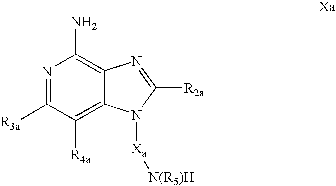
   wherein R₂ₐ, R₃ₐ, R₄ₐ, R₅, and Xₐ are as defined above; and
(viii-a) reacting a compound of the Formula Xa with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁C(O)—N═C═O, R₁—S(O)₂—N═C═O, R₁—N═C═S, R₁—C(O)—N═C═S, R₁—S(O)₂—N═C═S, R₁—S(O)₂—Cl, Cl—R₈—S(O)₂Cl, R₁—S(O)₂OS(O)₂—R₁, and R₁(R₇)N—S(O)₂Cl; wherein R₁, R₇, and R₈ are as defined above to provide a compound of Formula Ia or a pharmaceutically acceptable salt thereof defined above.

In some embodiments, a process (III-b) comprises the process (III) further comprising one or more steps selected from steps (iv), (v), and (vi) described above and (vii-a), (vii-b), and (viii-b):
(vii-a) reacting a compound of the Formula VIII defined above with triphenylphosphine to provide an N-triphenylphosphinyl compound of Formula IX 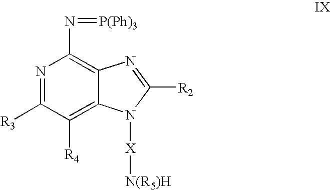
   wherein R₂, R₃, R₄, R₅, and X are as defined above;
(vii-b) hydrolyzing an N-triphenylphosphinyl compound of the Formula IX to provide a compound of Formula X 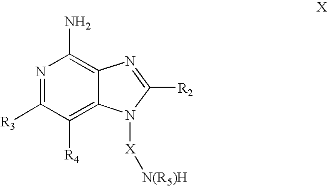
   wherein R₂, R₃, R₄, R₅, and X are as defined above; and
(viii-b) reacting a compound of the Formula X with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl; wherein R₁, R₇, and R₈ are as defined above to provide a compound of Formula I or a pharmaceutically acceptable salt thereof defined above.

In some embodiments, a process (III-c) comprises the process (III) further comprising one or more steps selected from steps (iv), (v), and (vi) described above and (viii-c) and (vii-2):
(viii-c) reacting a compound of the Formula VIIIa defined above with a compound selected from R₁ₐ—C(O)Cl, R₁ₐ(R₇)N—C(O)Cl, Cl—R₈—C(O)Cl, R₁ₐ—C(O)OC(O)—R₁ₐ, R₁ₐ(R₇)N—C(O)OC(O)—N(R₇)R₁ₐ, R₁ₐ—N═C═O, R₁ₐ—C(O)—N═C═O, R₁ₐ—S(O)₂—N═C═O, R₁ₐ—N═C═S, R₁ₐ—C(O)—N═C═S, R₁ₐ—S(O)₂—N═C═S, R₁ₐ—S(O)₂Cl, Cl—R₈—S(O)₂Cl, R₁ₐ—S(O)₂OS(O)₂—R₁ₐ, and R₁ₐ(R₇)N—S(O)₂Cl; wherein R₁ₐ, R₇, and R₈ are as defined above to provide a compound of Formula XIa defined above;
(vii-2) reductively removing the tetrazolo ring from the compound of Formula XIa to provide a 1H-imidazo[4,5-c]pyridin-4-amine compound of Formula Ib or a pharmaceutically acceptable salt thereof defined above.

In some embodiments, a process (III-d) comprises the process (III) further comprising one or more steps selected from steps (iv), (v), and (vi) described above and (viii-d), (vii-a-1), and (vii-b-1):
(viii-d) reacting a compound of the Formula VIII defined above with a compound selected from R₁—C(O)Cl, R₁(R₇)N—C(O)Cl, Cl—R₈—C(O)Cl, R—C(O)OC(O)—R₁, R₁(R₇)N—C(O)OC(O)—N(R₇)R₁, R₁—N═C═O, R₁—C(O)—N═C═O, R₁—S(O)₂-N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl; wherein R₁, R₇, and R₈ are as defined above; to provide a compound of Formula XI defined above;
(vii-a-1) reacting a compound of the Formula XI with triphenylphosphine to provide an N-triphenylphosphinyl compound of Formula XII defined above; and
(vii-b-1) hydrolyzing an N-triphenylphosphinyl compound of the Formula XII to provide a 1H-imidazo[4,5-c]pyridin-4-amine compound of Formula I or a pharmaceutically acceptable salt thereof defined above.

In certain embodiments, in step (iii) of processes (I-a-1), (I-b-1), (II-a-2), (II-b-2), (II-a-3), and (II-b-3) the compound of Formula III is reacted with an alkali metal azide in the presence of cerium III chloride.

In certain embodiments, the step (v) of processes (I-a-1), (I-b-1), (II-a-2), (II-b-2), (III-a), (II-b), (III-c), and (III-d) includes the steps of:
(v-a) reacting a compound of the Formula V with a carboxylic acid of the formula R₂CO₂H or the corresponding acyl halide to form a compound of the Formula VI 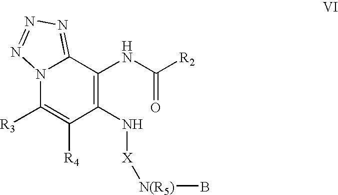
   wherein B, R₂, R₃, R₄, R₅, and X are as defined above; and
(v-b) subjecting the compound of the Formula VI to cyclization conditions, during step (v-a) or subsequent to the completion of step (v-a), to provide a compound of the Formula VII 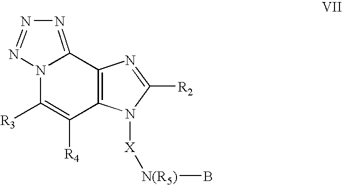
   wherein B, R₂, R₃, R₄, R₅, and X are as defined above.

In certain embodiments, in step (xiii) of processes (II-a-3) and (II-b-3) the compound of Formula XVI is reacted with an imidate of formula alkyl-O—C(═N)—R₂ as defined above to provide a compound of the Formula XI defined above.

In some embodiments of the above processes, R₁, R₂, R₃, R₄, R₁ₐ, R₂ₐ, R₃ₐ, R₄ₐ, R₅, B, X, Xₐ, Y and Z are independently selected as follows: R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇ is C₁₋₄ alkyl which may be interrupted by one or more heteroatoms, then R₁ and R₇ can join to form a ring having the structure 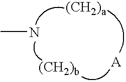
wherein A is selected from —O—, —S(O)₀₋₂—, —N(R₆)—, and —CH₂—; and a and b are independently integers from 1 to 4 with the proviso that a+b is less than or equal to 7; R₁ₐ is selected from C₁₋₄ branched alkyl, C₁₋₄ straight chain alkyl, cycloalkyl, and substituted cycloalkyl, or when R₁ₐ is C₁₋₄ straight chain alkyl, Z is —N(R₇)—, and R₇ is C₁₋₄ alkyl which may be interrupted by one or more heteroatoms, then R₁ₐ and R₇ can join to form a ring having the structure 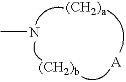
wherein A is selected from —O—, —S(O)₀₋₂—, —N(R₆)—, and —CH₂—; and a and b are independently integers from 1 to 4 with the proviso that a+b is less than or equal to 7; R₂ and R₂ₐ are selected from hydrogen, C₁₋₄ alkyl and C₁₋₄ alkylene-O—C₁₋₄ alkyl; R₃, R₃ₐ, R₄, and R₄ₐ are independently selected from hydrogen and methyl; R₅ is selected from hydrogen and C₁₋₄ alkyl; B is selected from t-butoxycarbonyl, iso-butoxycarbonyl, benzyloxycarbonyl, ethoxycarbonyl, methoxycarbonyl, benzoyl, pivaloyl, propionyl, acetyl, and phthalimide; X is selected from C₁₋₄ alkylene and C₁₋₄ alkenylene; Xₐ is C₁₋₄ alkylene; Y is selected from —CO— and —SO₂—; and Z is selected from a bond and —N(R₇)— wherein R₇ is hydrogen, C₁₋₄ alkyl which may be interrupted by one or more heteroatoms, or when R₇ is C₁₋₄ alkyl which may be interrupted by one or more heteroatoms R₇ is joined with R₁ or R₁ₐ as described above. In certain embodiments, R₁ is selected from methyl, phenyl, and cyclohexyl, or R₁ along with the nitrogen atom to which it is attached is joined with R₇ to form a morpholino ring; R₁ₐ is selected from methyl and cyclohexyl, or R₁ₐ along with the nitrogen atom to which it is attached is joined with R₇ to form a morpholino ring; R₂ and R₂ₐ are selected from hydrogen, methyl, ethyl, propyl, isopropyl, n-butyl, sec-butyl, isobutyl, tert-butyl, 2-methoxyethyl, ethoxymethyl, and cyclopropylmethyl; R₃, R₃ₐ, R₄, and R₄ₐ are methyl or R₃ and R₃ₐ are methyl and R₄ and R₄ₐ are hydrogen; R₅ is hydrogen; B is t-butoxycarbonyl; X and Xₐ are selected from ethylene, propylene (including straight chain and branched, for example, —CH₂—CH₂—CH₂—, —CH(CH₃)—CH₂—, —CH₂—CH(CH₃)—, —CH(CH₂CH₃)—, —C(CH₃)₂—), and butylene (including straight chain and branched, for example, —CH₂—CH₂—CH₂—CH₂—, —CH(CH₃)—CH₂—CH₂—, —CH₂—CH(CH₃)—CH₂—, —CH₂—CH₂—CH(CH₃)—, —C(CH₃)₂—CH₂—, —CH₂—C(CH₃)₂—, —CH(CH₂CH₃)—CH₂—, —CH₂—CH(CH₂CH₃)—, —C(CH₃)(CH₂CH₃)—, —CH(CH₂CH₂CH₃)—); Y is selected from —CO— and —SO₂—; Z is selected from a bond and —N(R₇)— wherein R₇ is hydrogen or R₇ along with the nitrogen atom to which it is attached is joined with R₁ or R₁ₐ to form a morpholino ring. In certain other embodiments, R₂ and R₂ₐ are methyl, ethyl, n-propyl, n-butyl, ethoxymethyl, or 2-methoxyethyl; and X and Xₐ are —CH₂CH₂—, or —CH₂C(CH₃)₂—.

The invention also provides novel compounds useful as intermediates in the preparation of the compounds of Formula I. These intermediates have the structural Formulas IV, V, VI, VII, VIII, XI, XIV, XV, and XVI.

One class of intermediate compounds has the Formula IV: 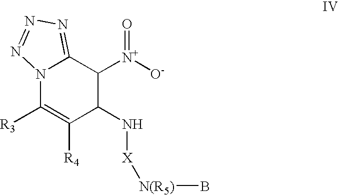
wherein
B is a protecting group for the —N(R₅)— amino group. Examples of B include but are not limited to t-butoxycarbonyl, iso-butoxycarbonyl, benzyloxycarbonyl, ethoxycarbonyl, methoxycarbonyl, benzoyl, pivaloyl, propionyl, acetyl, and phthalimide.
X is alkylene or alkenylene;
R₃ and R₄ are independently selected from hydrogen, alkyl, alkenyl, halogen, alkoxy, amino, alkylamino, dialkylamino, and alkylthio;
R₅ is H or C₁₋₁₀ alkyl, or when R₅ is C₁₋₁₀ alkyl, then R₅ can join with a carbon atom of X to form a ring having the structure 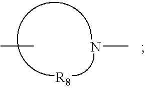
   and
R₈ is C₃₋₈ alkylene.

Another class of intermediate compounds has the Formula V 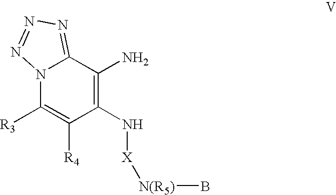
wherein B, X, R₃, R₄, and R₅ are as described above for the intermediate compound of Formula IV.

Another class of intermediate compounds has the Formula VI 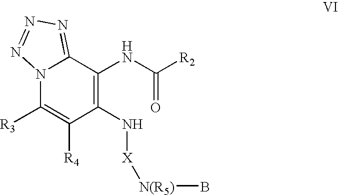
wherein
B, X, R₃, R₄, and R₅ are as described above for the intermediate compound of Formula IV;
R₂ is selected from:
   -hydrogen;
   -alkyl;
   -alkenyl;
   -aryl;
   -substituted aryl;
   -heteroaryl;
   -substituted heteroaryl;
   -alkylene-O-alkyl;
   -alkylene-S-alkyl;
   -alkylene-O-aryl;
   -alkylene-S-aryl;
   -alkylene-O-alkenyl;
   -alkylene-S- alkenyl; and
-alkyl or alkenyl substituted by one or more substituents selected from:
   —OH;
   -halogen;
   —CO—N(R₆)₂;
   —CS—N(R6)₂;
   —SO₂—N(R₆)₂;
   —NR₆—CO—C₁₋₁₀ alkyl;
   —NR₆—CS—C₁₋₁₀ alkyl;
   —NR₆—SO₂—C₁₋₁₀ alkyl;
   —CO—C₁₋₁₀ alkyl;
   —CO—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —CO-aryl;
   —CO-(substituted aryl);
   —CO-heteroaryl; and
   —CO-(substituted heteroaryl); and
each R₆ is independently H or C₁₋₁₀ alkyl.

Another class of intermediate compounds has the Formula VII 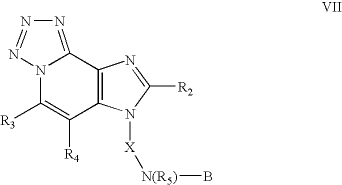
wherein B, X, R₂, R₃, R₄, and R₅ are as described above for the intermediate compound of Formula VI.

Another class of intermediate compounds has the Formula VIII 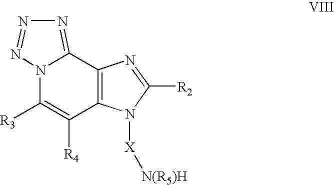
wherein X, R₂, R₃, R₄, and R₅ are as described above for the intermediate compound of Formula VI.

Another class of intermediate compounds has the Formula XI 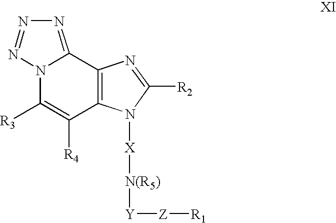
wherein
X, R₂, R₃, and R₄ are as described above for the intermediate compound of Formula VI;
Y is —CO—, —CS—, or —SO₂—;
Z is a bond, —N(R₇)—, —N(R₇)—CO—, or —N(R₇)—SO₂—; with the proviso that when Y is —SO₂—, then Z is a bond or —N(R₇)—;
R₁ is aryl, heteroaryl, heterocyclyl, alkyl or alkenyl, each of which may be unsubstituted or substituted by one or more substituents independently selected from:
   -alkyl;
   -alkenyl;
   -aryl;
   -heteroaryl;
   -heterocyclyl;
   -substituted cycloalkyl;
   -substituted aryl;
   -substituted heteroaryl;
   -substituted heterocyclyl;
   —O-alkyl;
   —O-(alkylene)₀₋₁-aryl;
   —O-(alkylene)₀₋₁-substituted aryl;
   —O-(alkylene)₀₋₁-heteroaryl;
   —O-(alkylene)₀₋₁-substituted heteroaryl;
   —O-(alkylene)₀₋₁-heterocyclyl;
   —O-(alkylene)₀₋₁-substituted heterocyclyl;
   —COOH;
   —CO—O-alkyl;
   —CO-alkyl;
   —S(O)₀₋₂-alkyl;
   —S(O)₀₋₂-(alkylene)₀₋₁-aryl;
   —S(O)₀₋₂-(alkylene)₀₋₁-substituted aryl;
   —S(O)₀₋₂-(alkylene)₀₋₁-heteroaryl;
   —S(O)₀₋₂-(alkylene)₀₋₁-substituted heteroaryl;
   —S(O)₀₋₂-(alkylene)₀₋₁-heterocyclyl;
   —S(O)₀₋₂-(alkylene)₀₋₁-substituted heterocyclyl;
   -(alkylene)₀₋₁-N(R₆)₂;
   -(alkylene)₀₋₁-NR₆—CO—O-alkyl;
   -(alkylene)₀₋₁-NR₆—CO-alkyl;
   -(alkylene)₀₋₁-NR₆—CO-aryl;
   -(alkylene)₀₋₁-NR₆—CO-substituted aryl;
   -(alkylene)₀₋₁-NR₆—CO-heteroaryl;
   -(alkylene)₀₋₁-NR₆—CO-substituted heteroaryl;
   —P(O)(O-alkyl)₂;
   —N₃;
   -halogen;
   -haloalkyl;
   -haloalkoxy;
   —CO-haloalkyl;
   —CO-haloalkoxy;
   —NO₂;
   —CN;
   —OH;
   —SH; and in the case of alkyl, alkenyl, and heterocyclyl, oxo;
R₅ is H or Cl₁₋₁₀ alkyl, or when R₅ is C₁₋₁₀ alkyl, then R₅ can join with a carbon atom of X to form a ring having the structure 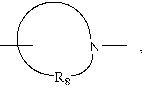
   or when R₅ is C₁₋₁₀ alkyl, R₁ is alkyl, and Z is a bond, then R₅ and R₁ can join to form a ring having the structure 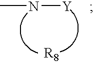
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 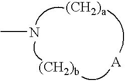
   wherein A is selected from —O—, —S(O)₀₋₂—, —N(R₆)—, and —CH₂—; and a and b are independently integers from 1 to 6 with the proviso that a+b is less than or equal to 7; and
R₈ is C₃₋₈ alkylene.

Another class of intermediate compounds has the Formula XIV 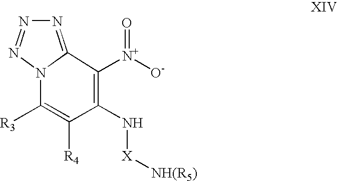
wherein
X is alkylene or alkenylene;
R₃ and R₄ are independently selected from hydrogen, alkyl, alkenyl, halogen, alkoxy, amino, alkylamino, dialkylamino, and alkylthio;
R₅ is H or C₁₋₁₀ alkyl, or when R₅ is C₁₋₁₀ alkyl, then R₅ can join with a carbon atom of X to form a ring having the structure 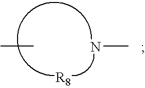
   and
R₈ is C₃₋₈ alkylene.

Another class of intermediate compounds has the Formula XV 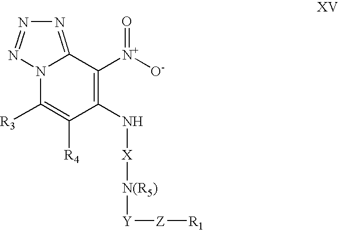
wherein
X, Y, Z, R₁, R₃, R₄, and R₅ are as described above for the intermediate compound of Formula XI.

Another class of intermediate compounds has the Formula XVI 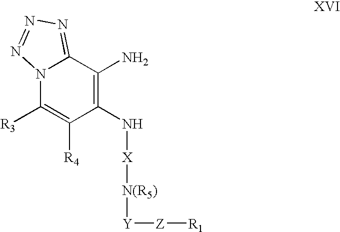
wherein
X, Y, Z, R₁, R₃, R₄, and R₅ are as described above for the intermediate compound of Formula XI;

In some embodiments of Formulas IV, V, VI, VII, VIII, XI, XIV, XV, and XVI, R₃ is methyl, R₄ is hydrogen or methyl, or R₃ and R4 are methyl. In certain embodiments of Formulas IV, V, VI, and VII, B is t-butoxycarbonyl.

In some embodiments of Formulas VI, VII, VIII, and XI, R₂ is selected from hydrogen, C₁₋₄ alkyl and C₁₋₄ alkylene-O—C₁₋₄ alkyl, and in certain other embodiments, R₂ is selected from hydrogen, methyl, ethyl, propyl, isopropyl, n-butyl, sec-butyl, isobutyl, tert-butyl, 2-methoxyethyl, ethoxymethyl, and cyclopropylmethyl.

In some embodiments of Formulas XI, XV, and XVI, R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇ is C₁₋₄ alkyl which may be interrupted by one or more heteroatoms, R₁ and R₇ can join to form a ring having the structure 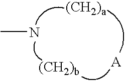
wherein A is selected from —O—, —S(O)₀₋₂—, —N(R₆)—, and —CH₂—; and a and b are independently integers from 1 to 4 with the proviso that a+b is less than or equal to 7. In certain other embodiments, R₁ is selected from methyl, phenyl, and cyclohexyl, or R₁ along with the nitrogen atom to which it is attached is joined with R₇ to form a morpholino ring.

As used herein, the terms “alkyl,” “alkenyl,” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene” and “alkenylene,” are the divalent forms of the “alkyl” and “alkenyl,” groups defined above. Likewise, “alkylenyl” and “alkenylenyl” are the divalent forms of the “alkyl” and “alkenyl” groups defined above. For example, an arylalkylenyl group comprises an alkylene moiety to which an aryl group is attached.

The term “haloalkyl” is inclusive of alkyl groups that are substituted by one or more halogen atoms, including perfluorinated groups. This is also true of other groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl.

The term “heteroatom” refers to the atoms O, S, or N.

The term “heteroaryl” includes aromatic rings or ring systems that contain at least one ring heteroatom (e.g., O, S, N). Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. Exemplary heterocyclic groups include pyrrolidinyl, tetrahydrofuranyl, morpholinyl, thiomorpholinyl, piperidinyl, piperazinyl, thiazolidinyl, imidazolidinyl, isothiazolidinyl, tetrahydropyranyl, quinuclidinyl, homopiperidinyl, homopiperazinyl, and the like.

Substituted cycloalkyl, substituted aryl, substituted heteroaryl, and substituted heterocyclyl groups can be substituted by one or more substituents independently selected from the group consisting of alkyl, alkoxy, methylenedioxy, ethylenedioxy, alkylthio, haloalkyl, haloalkoxy, haloalkylthio, halogen, nitro, hydroxy, mercapto, cyano, carboxy, formyl, aryl, aryloxy, arylthio, arylalkyleneoxy, arylalkylenethio, heteroaryl, heteroaryloxy, heteroarylthio, heteroarylalkyleneoxy, heteroarylalkylenethio, amino, alkylamino, dialkylamino, heterocyclyl, heterocyclylalkylenyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, aryloxycarbonyl, heteroaryloxycarbonyl, arylthiocarbonyl, heteroarylthiocarbonyl, alkanoyloxy, alkanoylthio, alkanoylamino, arylcarbonyloxy, arylcarbonylthio, alkylaminosulfonyl, alkylsulfonyl, arylsulfonyl, heteroarylsulfonyl, aryldiazinyl, alkylcarbonylamino, alkenylcarbonylamino, arylcarbonylamino, arylalkylenecarbonylamino, arylcarbonylaminoalkylenyl, heteroarylcarbonylamino, heteroarylalkylenecarbonylamino, alkylsulfonylamino, alkenylsulfonylamino, arylsulfonylamino, arylalkylenesulfonylamino, heteroarylsulfonylamino, heteroarylalkylenesulfonylamino, alkylaminocarbonylamino, alkenylaminocarbonylamino, arylaminocarbonylamino, arylalkyleneaminocarbonylamino, heteroarylaminocarbonylamino, heteroarylalkyleneaminocarbonylamino, and, in the case of heterocyclyl, oxo.

The tetrazoles of Formulas IV, V, VI, VII, VIII, XI, XIV, XV, and XVI can also exist in equilibrium with their 4-azido tautomeric form. The processes of the invention and Formulas IV, V, VI, VII, VIII, XI, XIV, XV, and XVI are inclusive of this tautomeric form.

The processes of the invention are useful, for example, for making compounds and salts of Formulas I, or for making intermediates which are useful for making such compounds and salts. Compounds and salts of Formula I are disclosed in U.S. Pat. Nos. 6,525,064; 6,545,016; and 6,545,017, and in International Publication WO 02/46194 as immune response modifiers due to their ability to induce cytokine biosynthesis and otherwise modulate the immune resonse when administered to animals. The compounds are useful in the treatment of a variety of conditions such as viral diseases and tumors that are responsive to such changes in the immune response.

Various aspects and embodiments of the invention are further described by the Examples, which are provided for illustration purposes only and are not intended to be limiting in any way.

### EXAMPLES

### Example 1

### tert-Butyl 2-[(5,6-dimethyl-8-nitrotetrazolo[1,5-a]pyridin-7-yl)amino]ethylcarbamate

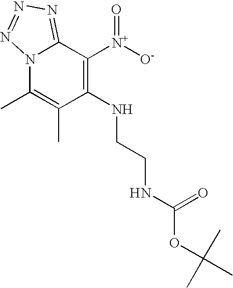 Part A

Under a nitrogen atmosphere, a solution of di-tert-butyl dicarbonate (100 g, 0.458 mol) in ethyl acetate (700 mL) was added to a solution of ethylenediamine (224 mL, 3.34 mol) in ethyl acetate (800 mL) over a period of one hour. During the addition, the temperature of the reaction rose from 20° C. to 34° C. The reaction was stirred overnight; a white precipitate formed, which was removed by filtration. The filtrate was concentrated under reduced pressure to provide a colorless oil, to which 750 mL of water was added. A white precipitate formed, which was removed by filtration and washed with water. The combined aqueous washings were extracted with dichloromethane (6×200 mL), and the combined extracts were dried over sodium sulfate, filtered, and concentrated under reduced pressure to provide 55.4 g of tert-butyl 2-aminoethylcarbamate as a colorless oil.

Part B

To a solution of 2,4-dichloro-5,6-dimethyl-3-nitropyridine (20.0 g, 90.5 mmol) in anhydrous N,N-dimethylformamide (200 mL) was added triethylamine (14.9 mL, 109 mmol) followed by tert-butyl 2-aminoethylcarbamate (17.4 g, 109 mmol) by dropwise addition under a nitrogen atmosphere. The reaction was heated at 60° C. overnight and became orange. The solvent was then removed under reduced pressure, and the resulting orange oil was dissolved in ethyl acetate (500 mL). The solution was washed with water (3×500 mL), dried over magnesium sulfate, filtered, and concentrated under reduced pressure to provide a yellow oil. The oil was triturated with methanol (100 mL) to form a bright yellow solid, which was isolated by filtration, washed with cold methanol, and dried under reduced pressure at room temperature to provide 22.7 g of tert-butyl 2-[(2-chloro-5,6-dimethyl-3-nitropyridin-4-yl)amino]ethylcarbamate as a bright yellow solid, m. p. 110-112° C.

Part C

Cerium (III) chloride heptahydrate (16.3 g, 43.8 mmol) was added to a solution of tert-butyl 2-[(2-chloro-5,6-dimethyl-3-nitropyridin-4-yl)amino]ethylcarbamate (30.2 g, 87.5 mmol) and sodium azide (11.4 g, 175 mmol) in a 9:1 solution of acetonitrile and water (600 mL), and the resulting mixture was heated at reflux for three days. The reaction was allowed to cool to room temperature, and a precipitate was removed by filtration and washed with additional acetonitrile. The solvents were removed under reduced pressure to provide an orange solid, which was partitioned between ethyl acetate (1.2 L) and water (500 mL). The organic layer was washed with water (3×400 mL), and the combined aqueous washings were extracted with ethyl acetate (200 mL). The combined organic solutions were dried over magnesium sulfate, filtered, and concentrated under reduced pressure to provide an orange solid. The solid was dissolved in a 10% solution of methanol in 2-propanol (550 mL) and cooled overnight in a refrigerator to provide yellow needles, which were isolated by filtration and washed with cold 2-propanol to yield 29.8 g of tert-butyl 2-[(5,6-dimethyl-8-nitrotetrazolo[1,5-a]pyridin-7-yl)amino]ethylcarbamate as yellow needles, m. p. 145-147° C.

### Example 2

### tert-Butyl 2-[(8-amino-5,6-dimethyltetrazolo[1,5-a]pyridin-7-yl)amino]ethylcarbamate

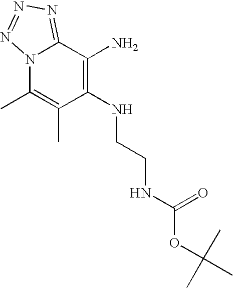

tert-Butyl 2-[(5,6-dimethyl-8-nitrotetrazolo[1,5-a]pyridin-7-yl)amino]ethylcarbamate (29.0 g, 82.5 mmol), anhydrous toluene (900 mL), and 2-propanol (100 mL) were added to a 2 L stainless steel Parr vessel. The starting material did not completely dissolve. The vessel was flushed with nitrogen, and 5% platinum on carbon (1.5 g) was added to the mixture. The vessel was placed under hydrogen pressure (30 psi, 2.1×10⁵ Pa) for four hours. The reaction mixture was filtered through a layer of CELITE filter aid, and the filter cake was washed with 2-propanol (500 mL) and ethanol (500 mL). The filtrate was concentrated under reduced pressure to yield 24.6 g of tert-butyl 2-[(8-amino-5,6-dimethyltetrazolo[1,5-a]pyridin-7-yl)amino]ethylcarbamate as a light green solid.

### Example 3

### tert-Butyl 2-(5,6,8-trimethyl-7H-imidazo[4,5-c]tetrazolo[1,5-a]pyridin-7-yl)ethylcarbamate

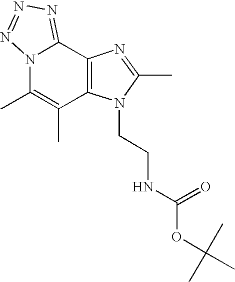

Under a nitrogen atmosphere, a mixture of tert-butyl 2-[(8-amino-5,6-dimethyltetrazolo[1,5-a]pyridin-7-yl)amino]ethylcarbamate (12.2 g, 38.0 mmol), triethyl orthoacetate (7.0 mL, 38.0 mmol), pyridine hydrochloride (1.2 g), and toluene (120 mL) was heated at reflux for 15 minutes. The reaction became homogeneous as it warmed to 60° C., and subsequently a white precipitate formed. The reaction was allowed to cool to room temperature, and the solvent was removed under reduced pressure. The resulting white solid was washed with water (500 mL), isolated by filtration, and dried in a vacuum oven overnight at 80° C. to provide 11.30 g of tert-butyl 2-(5,6,8-trimethyl-7H-imidazo[4,5-c]tetrazolo[1,5-a]pyridin-7-yl)ethylcarbamate, m.p. >250° C.

Example

### 2-(5,6,8-Trimethyl-7H-imidazo[4,5-c]tetrazolo[ 1,5-a]pyridin-7-yl)ethanamine trifluoroacetate

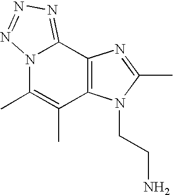

To a solution of trifluoroacetic acid (111 mL, 1.45 mol) in dichloromethane (200 mL) was slowly added a solution of tert-butyl 2-(5,6,8-trimethyl-7H-imidazo[4,5-c]tetrazolo[1,5-a]pyridin-7-yl)ethylcarbamate (10.0 g, 29.0 mmol) in dichloromethane (150 mL), and the reaction was stirred for 16 hours. The volatiles were then removed under reduced pressure, and additional dichloromethane (200 mL) was added and subsequently removed under reduced pressure to remove residual trifluoroacetic acid. The resulting white solid was stirred with methanol (200 mL) for one hour, isolated by filtration, washed with methanol, and dried in a vacuum oven overnight at 80° C. to provide 7.45 g of 2-(5,6,8-trimethyl-7H-imidazo[4,5-c]tetrazolo[1,5-a]pyridin-7-yl)ethanamine trifluoroacetate.

### Example 5

### N-[2-(5,6,8-Trimethyl-7H-imidazo[4,5-c]tetrazolo[1,5-a]pyridin-7-yl)ethyl]methanesulfonamide

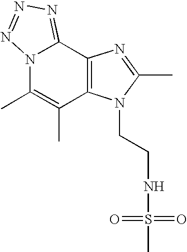

Under a nitrogen atmosphere, triethylamine (7.8 mL, 55.7 mmol) was slowly added to a mixture of 2-(5,6,8-trimethyl-7H-imidazo[4,5-c]tetrazolo[1,5-a]pyridin-7-yl)ethanamine trifluoroacetate (4.00 g, 11.1 mmol) and dichloromethane (320 mL). Methanesulfonyl chloride (0.90 mL, 11.7 mmol) was then added, and the reaction became homogeneous. The solution was stirred for one hour; a white precipitate formed. The solvent was removed under reduced pressure, and water (200 mL) and sodium carbonate (5 g) were added to the remaining white solid. The mixture was stirred for one hour, and the solid was isolated by filtration and dried in a vacuum oven at 80° C. to provide 3.16 g of N-[2-(5,6,8-trimethyl-7H-imidazo[4,5-c]tetrazolo [1,5-a]pyridin-7-yl)ethyl]methanesulfonamide as a white solid, m.p.>250° C.

### Example 6

### N-[2-(4-Amino-2,6,7-trimethyl-1H-imidazo[4,5-c]pyridin-1-yl)ethyl]methanesulfonamide

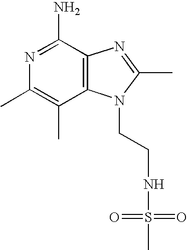

Under a nitrogen atmosphere, a solution of N-[2-(5,6,8-trimethyl-7H-imidazo[4,5-c]tetrazolo[1,5-a]pyridin-7-yl)ethyl]methanesulfonamide (3.00 g, 9.28 mmol) and triphenylphosphine (3.65 g, 13.9 mmol) in 1,2-dichlorobenzene (60 mL) was heated at reflux for 48 hours. The solvent was removed under reduced pressure to provide a brown oil, which was dissolved in methanol (60 mL). A solution of 1.0 M hydrochloric acid in diethyl ether (20 mL) was added to the methanol solution, and the reaction was heated at reflux for 24 hours. The reaction was allowed to cool to room temperature, and a brown solid formed, which was isolated by filtration and washed with cold methanol. The solid was dissolved in water (100 mL), and sodium carbonate (3 g) was added to the solution. A light brown solid was formed, which was isolated by filtration, washed with water, and dried in a vacuum oven overnight at 80° C. The crude product was purified by column chromatography on silica gel (eluting with 80:20 dichloromethane:methanol) and dried in a vacuum oven for two days at 90° C. to provide 1.02 g of N-[2-(4-amino-2,6,7-trimethyl-1H-imidazo[4,5-c]pyridin-1-yl)ethyl]methanesulfonamide as a white solid, m. p.>250° C. Analysis: Calculated for C₁₂H₁₉N₅O₂S: % C, 48.47; % H, 6.44; % N, 23.55. Found: % C, 48.17; % H, 6.35; % N, 23.26.

### Example 7

### tert-Butyl 2-({8-[(ethoxyacetyl)amino]-5,6-dimethyltetrazolo[1,5-a]pyridin-7-yl}amino)ethylcarbamate

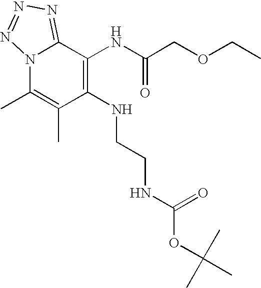

A solution of tert-butyl 2-[(8-amino-5,6-dimethyltetrazolo[1,5-a]pyridin-7-yl)amino]ethylcarbamate (24.3 g, 75.6 mmol) in anhydrous dichloromethane (650 mL) was cooled to 0° C. under a nitrogen atmosphere, and triethylamine (10.5 mL, 75.6 mmol) was added. A solution of ethoxyacetyl chloride (9.2 g, 75.6 mmol) in dichloromethane (100 mL) was added dropwise to the reaction. After the solution was stirred for two hours at 0° C., it was allowed to warm to room temperature overnight, washed with water (3×500 mL), dried over magnesium sulfate, filtered, and concentrated under reduced pressure to provide a colorless oil. The oil was triturated with diethyl ether (200 mL), and the resulting solid was isolated by filtration to yield 25.7 g of tert-butyl 2-({8-[(ethoxyacetyl)amino]-5,6-dimethyltetrazolo[1,5-a]pyridin-7-yl}amino)ethylcarbamate as a white solid, m. p. 138-142° C.

### Example 8

### tert-Butyl 2-[8-(ethoxymethyl)-5,6-dimethyl-7H-imidazo[4,5-c]tetrazolo[1,5-a]pyridin-7-yl]ethylcarbamate

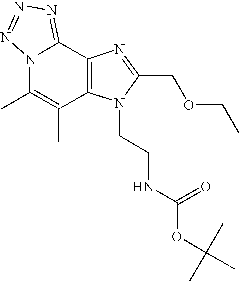

A solution of tert-butyl 2-({8-[(ethoxyacetyl)amino]-5,6-dimethyltetrazolo[1,5-a]pyridin-7-yl}amino)ethylcarbamate (25.5 g, 62.6 mmol) and pyridine hydrochloride (2.5 g) in pyridine (250 mL) was heated at reflux under a nitrogen atmosphere for 16 hours. The reaction was allowed to cool to room temperature; the solvent was then removed under reduced pressure. The resulting white solid was stirred with water (500 mL) for 30 minutes, isolated by filtration, and dried in a vacuum oven overnight at 80° C. to provide 21.2 g of tert-butyl 2-[8-(ethoxymethyl)-5,6-dimethyl-7H-imidazo[4,5-c]tetrazolo[1,5-a]pyridin-7-yl]ethylcarbamate as a white solid, m. p.197-199° C.

### Example 9

### 2-[8-(Ethoxymethyl)-5,6-dimethyl-7H-imidazo[4,5-c]tetrazolo[1,5-a]pyridin-7-yl]ethanamine trifluoroacetate

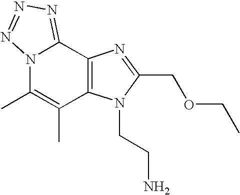

The reaction was carried out as described in Example 4 with the following exceptions. A solution of trifluoroacetic acid (150 mL, 1.95 mol) in dichloromethane (750 mL) was cooled to 0° C., and a solution of tert-butyl 2-[8-(ethoxymethyl)-5,6-dimethyl-7H-imidazo[4,5-c]tetrazolo[1,5-a]pyridin-7-yl]ethylcarbamate (21.0 g, 53.9 mmol) in dichloromethane (250 mL) was slowly added. The reaction was allowed to stir for four hours. After the volatiles were removed under reduced pressure, two additional portions of dichloromethane were added and subsequently removed under reduced pressure to provide a light brown solid. The solid was stirred with 2-propanol (400 mL) for one hour, isolated by filtration, and dried in a vacuum oven for four hours at 80° C. to provide 20.6 g of 2-[8-(ethoxymethyl)-5,6-dimethyl-7H-imidazo[4,5-c]tetrazolo[1,5-a]pyridin-7-yl]ethanamine trifluoroacetate as a white solid.

### Example 10

### N-{2-[8-(Ethoxymethyl)-5,6-dimethyl-7H-imidazo[4,5-c]tetrazolo[1,5-a]pyridin-7-yl]ethyl}methanesulfonamide

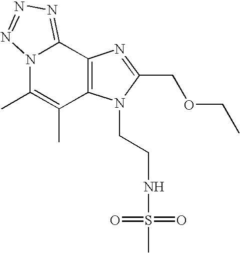

The reaction was carried out as described in Example 5 with the following exceptions. Triethylamine (4.73 mL, 34.1 mmol) was added to 2-[8-(ethoxymethyl)-5,6-dimethyl-7H-imidazo[4,5-c]tetrazolo[1,5-a]pyridin-7-yl]ethanamine trifluoroacetate (2.75 g, 6.82 mmol) in dichloromethane (275 mL), and the reaction became homogeneous after five minutes of stirring. Methanesulfonyl chloride (0.528 mL, 6.82 mmol) was then added. After the reaction was stirred for one hour, an analysis by high-performance liquid chromatography indicated the reaction was incomplete, and additional methanesulfonyl chloride (0.11 mL, 1.36 mmol) was added. The solution was stirred for 16 hours. The white solid isolated after removal of the solvent was stirred with water (150 mL) and sodium carbonate (5 g). After the solid was isolated, washed, and dried, 2.23 g of N-{2-[8-(ethoxymethyl)-5,6-dimethyl-7H-imidazo[4,5-c]tetrazolo[1,5-a]pyridin-7-yl]ethyl}methanesulfonamide was obtained as a white solid, m. p. 218-222° C.

### Example 11

### N-{2-[8-(Ethoxymethyl)-5,6-dimethyl-7H-imidazo[4,5-c]tetrazolo[1,5-a]pyridin-7-yl]ethyl}morpholine-4-carboxamide

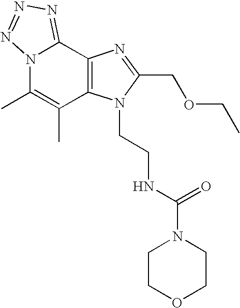

Under a nitrogen atmosphere, triethylamine (4.73 mL, 34.1 mmol) was added to a mixture of 2-[8-(ethoxymethyl)-5,6-dimethyl-7H-imidazo[4,5-c]tetrazolo[1,5-a]pyridin-7-yl]ethanamine trifluoroacetate (2.75 g, 6.82 mmol) in dichloromethane (275 mL), and the reaction became homogeneous. After the reaction was stirred for five minutes, 4-morpholinecarbonyl chloride (0.79 mL, 6.82 mmol) was added dropwise to the solution. The reaction was stirred for two days, and an analysis by high-performance liquid chromatography indicated that the reaction was incomplete. Additional 4-morpholinecarbonyl chloride (0.08 mL, 0.68 mmol) was added, and the reaction was stirred for three more days. The volatiles were removed under reduced pressure, and the resulting white solid was dissolved in dichloromethane (250 mL). The organic solution was washed with water (3×100 mL), dried over magnesium sulfate, filtered, and concentrated under reduced pressure. The resulting solid was dried in a vacuum oven overnight at 80° C. to provide 2.55 g of N-{2-[8-(ethoxymethyl)-5,6-dimethyl-7H-imidazo[4,5-c]tetrazolo[1,5-a]pyridin-7-yl]ethyl}morpholine-4-carboxamide as a white solid, m. p. 205-207° C.

### Example 12

### N-{2-[8-(Ethoxymethyl)-5,6-dimethyl-7H-imidazo[4,5-c]tetrazolo[1,5-a]pyridin-7-yl]ethyl}-N′-phenylurea

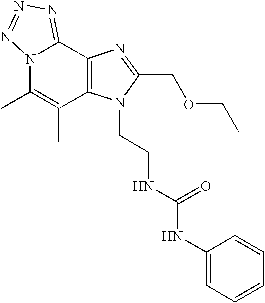

Under a nitrogen atmosphere, triethylamine (1.0 mL, 7.44 mmol) was added to a mixture of 2-[8-(ethoxymethyl)-5,6-dimethyl-7H-imidazo[4,5-c]tetrazolo[1,5-a]pyridin-7-yl]ethanamine trifluoroacetate (3.00 g, 7.44 mmol) in dichloromethane (300 mL), and the reaction became homogeneous. Phenyl isocyanate (0.85 mL, 7.81 mmol) was then added. The resulting solution was stirred for two hours, washed with water (3×500 mL), dried over magnesium sulfate, filtered, and concentrated under reduced pressure to provide 3.0 g of N-{2-[8-(ethoxymethyl)-5,6-dimethyl-7H-imidazo[4,5-c]tetrazolo[1,5-a]pyridin-7-yl]ethyl}-N′-phenylurea as a white solid, m. p.>250° C. MS(CI) m/z 409 (M+H).

### Example 13

### N-{2-[8-(Ethoxymethyl)-5,6-dimethyl-7H-imidazo [4,5 -c]tetrazolo[1,5-a]pyridin-7-yl]ethyl}-2-methylpropanamide

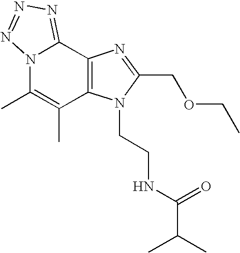

Under a nitrogen atmosphere, triethylamine (12.1 mL, 86.8 mmol) was added to a mixture of 2-[8-(ethoxymethyl)-5,6-dimethyl-7H-imidazo[4,5-c]tetrazolo[1,5-a]pyridin-7-yl]ethanamine trifluoroacetate (3.50 g, 8.68 mmol) in dichloromethane (175 mL), and the reaction became homogeneous after five minutes of stirring. 2-Methylpropanoyl chloride (0.95 mL, 9.11 mmol) was then added by dropwise addition, and the resulting slightly yellow solution was stirred for one hour. The volatiles were removed under reduced pressure, and the resulting yellow oil was partitioned between dichloromethane (250 mL) and 5% aqueous sodium carbonate (100 mL). The aqueous layer was extracted with dichloromethane (3×250 mL), and the combined organic extracts were dried over magnesium sulfate, filtered, and concentrated under reduced pressure to provide 3.0 g of a white solid. This solid was recrystallized from 2-propanol (6.7 mL/g) to provide 2.50 g of N-{2-[8-(ethoxymethyl)-5,6-dimethyl-7H-imidazo[4,5-c]tetrazolo[1,5-a]pyridin-7-yl]ethyl}-2-methylpropanamide as off-white needles, m. p. 179-181° C. Analysis: Calculated for C₁₇H₂₅N₇O₂: % C, 56.81; % H, 7.01; % N, 27.28. Found: % C, 56.73; % H, 6.99; % N, 27.47.

### Example 14

### N-{2-[4-Amino-2-(ethoxymethyl)-6,7-dimethyl-1H-imidazo[4,5-c]pyridin-1-yl]ethyl}methanesulfonamide

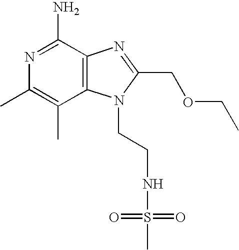

N-{2-[8-(Ethoxymethyl)-5,6-dimethyl-7H-imidazo[4,5-c]tetrazolo[1,5-a]pyridin-7-yl]ethyl}methanesulfonamide (2.00 g, 5.44 mmol), trifluoroacetic acid (20 mL), and platinum (IV) oxide (0.20 g) were added to a stainless steel Parr vessel, which was then placed under hydrogen pressure (50 psi, 3.4×10⁵ Pa). For the first six hours, the vessel was flushed with hydrogen every two hours and then maintained under hydrogen pressure (50 psi, 3.4×10⁵ Pa) for two days. The reaction mixture was filtered through a layer of CELITE filter aid, and the filter cake was washed with additional trifluoroacetic acid. The filtrate was concentrated under reduced pressure to yield a light brown oil, which was dissolved in water (25 mL). Sodium carbonate was added to this solution until it exhibited pH 12, and a white solid formed that was isolated by filtration and washed with water. The solid was dissolved in ethanol (50 mL) with heating, and the resulting solution was allowed to cool to room temerperature before a 1.0 M solution of hydrochloric acid in diethyl ether (5.4 mL) was added. After the solution was stirred for one hour, the salt precipitated, and the mixture was cooled to near 0° C. The salt was isolated by filtration, washed with cold ethanol, dried, and then dissolved in water (25 mL). Sodium carbonate (1.5 g) was added to the resulting solution until it exhibited pH 12, and a white solid formed that was isolated by filtration, washed with water, and dried in a vacuum oven for two hours at 80° C. to provide 0.87 g of N-{2-[4-amino-2-(ethoxymethyl)-6,7-dimethyl-1H-imidazo[4,5-c]pyridin-1-yl]ethyl}methanesulfonamide as a white solid, m. p. 185-187 ° C. Analysis: Calculated for C₁₄H₂₃N₅O₃S: % C, 49.25; % H, 6.79; % N, 20.51. Found: % C, 49.32; % H, 6.74; % N, 20.61.

### Example 15

### N-{2-[4-Amino-2-(ethoxymethyl)-6,7-dimethyl-1H-imidazo[4,5-c]pyridin-1-yl]ethyl}morpholine-4-carboxamide

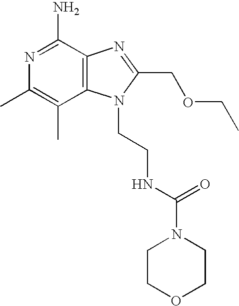

The reaction was carried out as described in Example 14 with the following exceptions. N-{2-[8-(Ethoxymethyl)-5,6-dimethyl-7H-imidazo[4,5-c]tetrazolo[1,5-a]pyridin-7-yl]ethyl}morpholine-4-carboxamide (1.85 g, 4.60 mmol), trifluoroacetic acid (18 mL), and platinum (IV) oxide (0.092 g) were added to the Parr vessel. The brown oil, isolated after concentration of the reaction mixture, was dissolved in 37% aqueous hydrochloric acid (30 mL), and the resulting solution was stirred for 15 minutes. The pH of the solution was adjusted to 12 with the addition of sodium carbonate, and the resulting solution was extracted with dichloromethane (3×300 mL). The combined extracts were dried over magnesium sulfate, filtered, and concentrated under reduced pressure to provide a white solid that was stirred with diethyl ether and isolated by filtration. The solid was dissolved in 2-propanol (50 mL) with heating, and the resulting solution was allowed to cool to room temperature before a 1.0 M solution of hydrochloric acid in diethyl ether (4.0 mL) was added. The salt that formed was isolated by filtration and dissolved in water (25 mL). The pH of the solution was adjusted to 12 with the addition of sodium carbonate, and the resulting white precipitate was isolated by filtration, washed with water, and dried in vacuum oven at 90° C. to provide 1.05 g of N-{2-[4-amino-2-(ethoxymethyl)-6,7-dimethyl-1H-imidazo[4,5-c]pyridin-1-yl]ethyl}morpholine-4-carboxamide as a white solid, m. p. 179-181° C. Analysis: Calculated for C₁₈H₂₈N₆O₃.0.25 H₂O: % C, 56.76; % H, 7.54; % N, 22.06. Found: % C, 56.74; % H, 7.60; % N, 22.18.

### Example 16

### N-{2-[4-Amino-2-(ethoxymethyl)-6,7-dimethyl-1H-imidazo[4,5-c]pyridin-1-yl]ethyl}-2-methylpropanamide

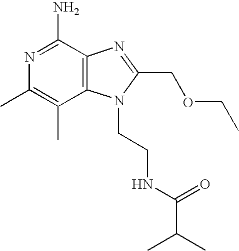

The reaction was carried out as described in Example 14 with the following exceptions. N-{2-[8-(Ethoxymethyl)-5,6-dimethyl-7H-imidazo[4,5-c]tetrazolo[1,5-a]pyridin-7-yl]ethyl}-2-methylpropanamide (1.85 g, 5.15 mmol), trifluoroacetic acid (20 mL), and platinum (IV) oxide (0.09 g) were added to the Parr vessel. The reaction was maintained under hydrogen pressure (50 psi, 3.4×10⁵ Pa) overnight. The light brown oil, isolated after concentration of the reaction mixture, was dissolved in 37% aqueous hydrochloric acid (30 mL). The pH of the solution was adjusted to 12 with the addition of sodium carbonate, and the resulting solution was extracted with dichloromethane (3×300 mL). The combined extracts were dried over magnesium sulfate, filtered, and concentrated under reduced pressure to provide a colorless oil, which was triturated with diethyl ether to form crystals. The crystals were recrystallized from toluene (60 mL) to provide 1.4 g of white needles, which were further purified by column chromatography on silica gel (eluting with 95:5 dichloromethane:methanol) and dried in a vacuum oven at 85° C. to provide 1.13 g of N-{2-[4-amino-2-(ethoxymethyl)-6,7-dimethyl-1H-imidazo[4,5-c]pyridin-1-yl]ethyl}-2-methylpropanamide as a white solid, m. p. 172-174° C. Analysis: Calculated for C₁₇H₂₇N₅O₂: % C, 61.24; % H, 8.16; % N, 21.00. Found: % C, 61.01; % H, 8.37; % N, 20.96.

### Example 17

### 1-(2-Aminoethyl)-2-(ethoxymethyl)-6,7-dimethyl-1H-imidazo[4,5-c]pyridin-4-amine

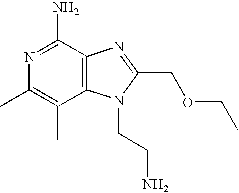

The reaction was carried out as described in Example 14 with the following exceptions. 2-[8-(Ethoxymethyl)-5,6-dimethyl-7H-imidazo[4,5-c]tetrazolo[1,5-a]pyridin-7-yl]ethanamine trifluoroacetate (14.2 g, 35.2 mmol), trifluoroacetic acid (105 mL), and platinum (IV) oxide (0.73 g) were added to the Parr vessel. For the first eight hours, the vessel was flushed with hydrogen every two hours and then maintained under hydrogen pressure (50 psi, 3.4×10⁵ Pa) overnight. The brown oil, isolated after concentration of the reaction mixture, was dissolved in 37% aqueous hydrochloric acid (25 mL). The pH of the solution was adjusted to 12 with the addition of sodium carbonate, and the resulting solution was extracted with chloroform for two days using a continuous extractor. The chloroform solution was dried over magnesium sulfate, filtered, and concentrated under reduced pressure to provide 8.45 g of 1-(2-aminoethyl)-2-(ethoxymethyl)-6,7-dimethyl-1H-imidazo[4,5-c]pyridin-4-amine as a white solid, m. p. 163-166° C.

### Example 18

### N-{2-[4-Amino-2-(ethoxymethyl)-6,7-dimethyl-1H-imidazo[4,5-c]pyridin-1-yl]ethyl}-N′-phenylurea

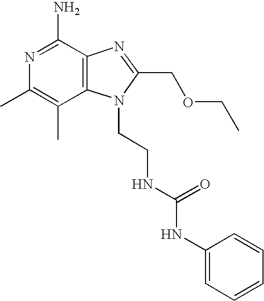

Under a nitrogen atmosphere, a solution of 1-(2-aminoethyl)-2-(ethoxymethyl)-6,7-dimethyl-1H-imidazo[4,5-c]pyridin-4-amine (1.75 g, 6.65 mmol) in anhydrous dichloromethane (175 mL) was cooled to 0° C. Phenyl isocyanate (0.73 mL, 6.65 mmol) was added dropwise to the solution, and the resulting solution was stirred for one hour. The solvent was removed under reduced pressure to provide a white solid (2.55 g), which was heated in methanol (50 mL). An insoluble impurity was removed by filtration, and the filtrate was concentrated under reduced pressure to provide 2.05 g of a white solid that was dissolved in 2-propanol (25 mL) with heating. To the resulting solution was added a 1.0 M solution of hydrochloric acid in diethyl ether (5.2 mL), and the resulting salt was isolated by filtration and washed sequentially with cold 2-propanol and diethyl ether. The salt was partitioned between 10% aqueous sodium carbonate and dichloromethane. The aqueous layer was extracted with dichloromethane (3×250 mL), and the combined organic solutions were dried over magnesium sulfate, filtered, and concentrated under reduced pressure to provide a colorless oil, which was triturated with diethyl ether to form crystals. The crystals were purified by column chromatography on silica gel (eluting with 90:10 dichloromethane:methanol) to provide 1.33 g of N-{2-[4-amino-2-(ethoxymethyl)-6,7-dimethyl-1H-imidazo[4,5-c]pyridin-1-yl]ethyl}-N′-phenylurea as a white solid, m. p. 173-175° C. The solid first melted at 110° C. and recrystallized. Analysis: Calculated for C₂₀H₂₆N₆O₂.0.25 H₂O: % C, 62.08; % H, 6.90; % N, 21.72. Found: % C, 62.27; % H, 6.61; % N, 21.80.

### Example 19

### N-{2-[4-Amino-2-(ethoxymethyl)-6,7-dimethyl-1H-imidazo[4,5-c]pyridin-1-yl]ethyl}-N-cyclohexylurea

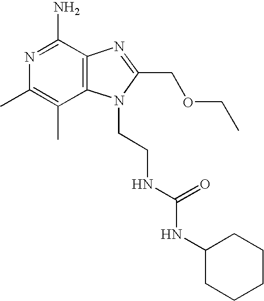

Under a nitrogen atmosphere, a solution of 1-(2-aminoethyl)-2-(ethoxymethyl)-6,7-dimethyl-1H-imidazo[4,5-c]pyridin-4-amine (1.75 g, 6.65 mmol) in anhydrous dichloromethane (175 mL) was cooled to 0° C. Cyclohexyl isocyanate (0.85 mL, 6.65 mmol) was added dropwise to the solution, and the resulting solution was stirred for one hour then allowed to warm to room temperature. An analysis by high-performance liquid chromatography indicated that the reaction was incomplete. Additional cyclohexyl isocyanate (0.085 mL, 0.66 mmol) was added, and the reaction was stirred overnight. The solvent was removed under reduced pressure, and the resulting oil was dissolved in 2-propanol (20 mL). A 1.0 M solution of hydrochloric acid in diethyl ether (6.6 mL) was added, and a white salt formed. The mixture was stirred for one hour, and the salt was then isolated by filtration and washed sequentially with cold 2-propanol and diethyl ether. The salt was dissolved in water (30 mL), and the pH of the resulting solution was adjusted to 12 using sodium carbonate. A white oil formed, which slowly solidified. The solid was isolated by filtration and dried in a vacuum oven overnight at 80° C. to provide 1.74 g of N-{2-[4-amino-2-(ethoxymethyl)-6,7-dimethyl-1H-imidazo[4,5-c]pyridin-1-yl]ethyl}-N′-cyclohexylurea as a white solid, m. p. 188-190° C. Analysis: Calculated for C₂₀H₃₂N₆O₂: % C, 61.83; % H, 8.30; % N, 21.63. Found: % C, 61.57; % H, 8.07; % N, 21.45.

### Example 20

### N-{2-[4-Amino-2-(ethoxymethyl)-6,7-dimethyl-1H-imidazo[4,5-c]pyridin-1-yl]ethyl}benzamide hydrochloride

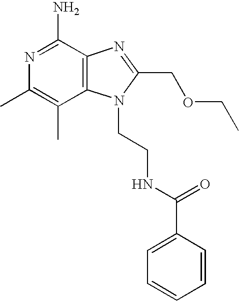

Under a nitrogen atmosphere, a solution of 1-(2-aminoethyl)-2-(ethoxymethyl)-6,7-dimethyl-1H-imidazo[4,5-c]pyridin-4-amine (1.50 g, 5.70 mmol) in anhydrous dichloromethane (150 mL) was cooled to 0° C.; triethylamine (0.78 mL, 5.60 mmol) was then added. Benzoyl chloride (0.66 mL, 5.70 mmol) was added dropwise, and the reaction was stirred for three hours. The reaction solution was washed with 10% aqueous sodium hydroxide (150 mL), and this solution was then extracted with dichloromethane (150 mL). The combined organic solutions were dried over magnesium sulfate, filtered, and concentrated under reduced pressure to provide a foamy solid, which was purified by column chromatography on silica gel (eluting with 85:15 dichloromethane:methanol). The resulting solid (1.4 g) was dissolved in ethanol (20 mL) and methanol (20 mL), and a 1.0 M solution of hydrochloric acid in diethyl ether (3.8 mL) was added to form a white salt. The mixture was stirred for one hour, and the mixture was stored in a freezer overnight. The salt was then isolated by filtration, washed sequentially with cold ethanol and diethyl ether, and dried in a vacuum oven overnight at 80° C. to provide 1.21 g of N-{2-[4-amino-2-(ethoxymethyl)-6,7-dimethyl-1H-imidazo[4,5-c]pyridin-1-yl]ethyl}benzamide hydrochloride as a white powder, m. p.>250° C. Analysis: Calculated for C₂₀H₂₅N₅O₂.HCl: % C, 59.47; % H, 6.49; % N, 17.34. Found: % C, 59.38; % H, 6.40; % N, 17.32.

### Example 21

### N-{2-[4-Amino-2-(ethoxymethyl)-6,7-dimethyl-1H-imidazo[4,5-c]pyridin-1-yl]ethyl}cyclohexanecarboxamide hydrochloride

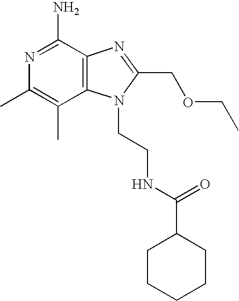

Under a nitrogen atmosphere, a solution of 1-(2-aminoethyl)-2-(ethoxymethyl)-6,7-dimethyl-1H-imidazo[4,5-c]pyridin-4-amine (1.50 g, 5.70 mmol) in anhydrous dichloromethane (150 mL) was cooled to 0° C.; triethylamine (0.79 mL, 5.70 mmol) was then added. Cyclohexylcarbonyl chloride (0.76 mL, 5.70 mmol) was added dropwise, and the reaction was stirred overnight. An analysis by high-performance liquid chromatography indicated that the reaction was not complete, and additional cyclohexylcarbonyl chloride (0.076, 0.57 mmol) was added. The resulting solution was stirred for one hour. The reaction solution was washed with 10% aqueous sodium hydroxide (200 mL), and this solution was then extracted with dichloromethane (200 mL). The combined organic solutions were dried over magnesium sulfate, filtered, and concentrated under reduced pressure to provide 2.04 g of a foamy solid. The solid was dissolved in 2-propanol (25 mL) with heating, and a 1.0 M solution of hydrochloric acid in diethyl ether (5.6 mL) was added to form a white salt. The mixture was stirred for one hour, and the salt was then isolated by filtration, washed sequentially with cold 2-propanol and diethyl ether, and dried in a vacuum oven overnight at 80° C. to provide 1.76 g of N-{2-[4-amino-2-(ethoxymethyl)-6,7-dimethyl-1H-imidazo[4,5-c]pyridin-1-yl]ethyl}cyclohexanecarboxamide hydrochloride as a white solid, m. p. 244-246° C. Analysis: Calculated for C₂₀H₃₁N₅O₂.HCl: % C, 58.60; % H, 7.87; % N, 17.03. Found: % C, 58.46; % H, 8.00; % N, 17.01.

### Example 22

### tert-Butyl 2-[(5,6-dimethyl-8-nitrotetrazolo[1,5-a]pyridin-7-yl)amino]-1,1-dimethylethylcarbamate

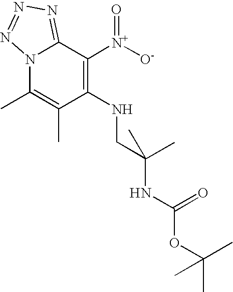 Part A

1,2-Diamino-2-methylpropane (11.4 mL, 108 mmol) was added dropwise to a cooled solution of 2,4-dichloro-5,6-dimethyl-3-nitropyridine (20.0 g, 90.4 mmol) and triethylamine (15.2 mL, 108 mmol) in anhydrous N,N-dimethylformamide (200 mL). The reaction mixture was heated at 60° C. overnight. The reaction mixture was concentrated under reduced pressure to provide an orange oil. The oil was partitioned between ethyl acetate and aqueous sodium bicarbonate (500 mL). The aqueous layer was extracted with ethyl acetate (3×500 mL). The organics were combined, dried over magnesium sulfate, filtered, and concentrated under reduced pressure to provide an orange oil. The oil was triturated with isopropanol (50 mL) and then chilled overnight in a freezer. The resulting solid was isolated by filtration and rinsed with a minimum amount of cold isopropanol to provide 14.0 g of N¹-(2-chloro-5,6-dimethyl-3-nitropyridin-4-yl)-2-methylpropane-1,2-diamine as a light orange crystalline solid, mp 89.0-94.0° C.

Part B

Sodium hydroxide (18.5 mL of 2N) was added dropwise to a chilled (ice bath) mixture of N¹-(2-chloro-5,6-dimethyl-3-nitropyridin-4-yl)-2-methylpropane-1,2-diamine (10 g, 36.7 mmol) and tetrahydrofuran (50 mL). A solution of di-tert-butyl dicarbonate (8.0 g, 36.7 mmol) in tetrahydrofuran (50 mL) was added dropwise over 30 minutes. After 2 hours the reaction mixture was allowed to warm to ambient temperature and then left overnight. Analysis by high performance liquid chromatography (HPLC) indicated that the reaction was not complete so additional di-tert-butyl dicarbonate (0.05 eq) was added. After 2 hours the reaction mixture was concentrated under reduced pressure. The residue was partitioned between water (100 mL) and ethyl acetate (100 mL). The aqueous layer was extracted with ethyl acetate (2×100 mL). The organics were combined, dried over magnesium sulfate, filtered, and concentrated under reduced pressure to provide a yellow oil. The oil was dissolved with heating in hexane (75 mL), cooled to ambient temperature, and then placed in a freezer overnight. The resulting solid was isolated by filtration to provide 12.0 g of tert-butyl 2-[(2-chloro-5,6-dimethyl-3-nitropyridin-4-yl)amino]-1,1-dimethylethylcarbamate as a yellow crystalline solid, mp 97.0-100.0° C.

Part C

Using the general method of Example 1 Part C, tert-butyl 2-[(2-chloro-5,6-dimethyl-3-nitropyridin-4-yl)amino]-1,1-dimethylethylcarbamate (70.0 g, 188 mmol) was reacted with sodium azide (24.4 g, 375 mmol).in the presence of cerium (III) chloride heptahydrate (35.0 g, 94 mmol). The crude product was recrystallized from toluene to provide 66 g of tert-butyl 2-[(5,6-dimethyl-8-nitrotetrazolo[1,5-a]pyridin-7-yl)amino]-1,1-dimethylethylcarbamate as a yellow powder, mp 153.0-155.0° C.

### Example 23

### tert-Butyl 2-[(8-amino-5,6-dimethyltetrazolo[1,5-a]pyridin-7-yl)amino]-1,1-dimethylethylcarbamate

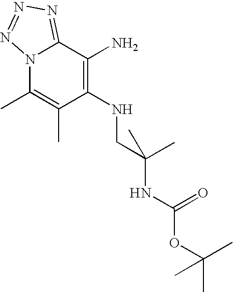

Using the general method of Example 2, tert-butyl 2-[(5,6-dimethyl-8-nitrotetrazolo[1,5-a]pyridin-7-yl)amino]-1,1-dimethylethylcarbamate (40.0 g, 105 mmol) was reduced to provide 35.6 g of tert-butyl 2-[(8-amino-5,6-dimethyltetrazolo[1,5-a]pyridin-7-yl)amino]-1,1-dimethylethylcarbamate as a light brown solid.

### Example 24

### tert-Butyl 2-(8-butyl-5,6-dimethyl-7H-imidazo[4,5-c]tetrazolo[1,5-a]pyridin-7-yl)-1,1-dimethylethylcarbamate

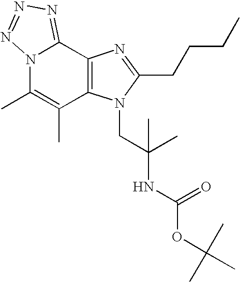

Trimethyl orthovalerate (18.4 mL, 107 mmol) and pyridine hydrochloride (3.5 g) were added sequentially to a mixture of tert-butyl 2-[(8-amino-5,6-dimethyltetrazolo[1,5-a]pyridin-7-yl)amino]-1,1-dimethylethylcarbamate (35.5 g, 102 mmol) and anhydrous toluene (700 mL) and the reaction mixture was heated to reflux. After about 1 hour additional pyridine hydrochloride (6 g ) was added and the reaction mixture was heated at reflux overnight. The reaction mixture was allowed to cool to ambient temperature and then concentrated under reduced pressure to provide a brown oil. The oil was partitioned between dichloromethane (500 mL) and 5% sodium hydroxide (400 mL). The aqueous layer was extracted with dichloromethane (2×300 mL). The organics were combined, dried over magnesium sulfate, filtered, and concentrated under reduced pressure to provide about 40 g of a foamy brown solid. The solid was triturated with 30/70 ethyl acetate/hexanes (300 mL). The resulting solid was isolated by filtration and washed with cold 30/70 ethyl acetate/ hexanes to provide 10.6 g of tert-butyl 2-(8-butyl-5,6-dimethyl-7H-imidazo[4,5-c]tetrazolo[1,5-a]pyridin-7-yl)-1,1-dimethylethylcarbamate as a white solid, mp 179.0-181.0° C.

### Example 25

### 1-(8-Butyl-5,6-dimethyl-7H-imidazo[4,5-c]tetrazolo[1,5-a]pyridin-7-yl)-2-methylpropan-2-amine

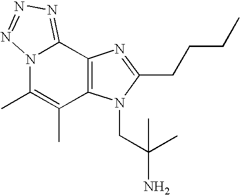

Tert-butyl 2-(8-butyl-5,6-dimethyl-7H-imidazo[4,5-c]tetrazolo[1,5-a]pyridin-7-yl)-1,1-dimethylethylcarbamate (10.4 g, 25.0 mmol) was added in portions over a period of 5 minutes to a chilled (ice bath) solution of hydrochloric acid in ethanol (50 mL of 3.6M). The reaction mixture was heated at reflux for 1 hour, allowed to cool to ambient temperature, and then concentrated under reduced pressure to provide a clear oil. The oil was partitioned between dichloromethane (200 mL) and 10% sodium hydroxide (200 mL). The aqueous layer was extracted with dichloromethane (2×200 mL). The organics were combined, dried over magnesium sulfate, filtered, and concentrated under reduced pressure to provide 7.76 g of 1-(8-butyl-5,6-dimethyl-7H-imidazo[4,5-c]tetrazolo[1,5-a]pyridin-7-yl)-2-methylpropan-2-amine as a white solid, mp 144.0-147.0° C.

### Example 26

### N-[2-(8-Butyl-5,6-dimethyl-7H-imidazo[4,5-c]tetrazolo[1,5-a]pyridin-7-yl)-1,1-dimethylethyl]methanesulfonamide

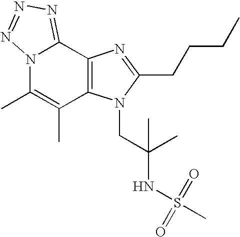

Triethylamine (0.93 mL, 6.66 mmol) was added dropwise to a chilled (ice bath) mixture of 1-(8-butyl-5,6-dimethyl-7H-imidazo[4,5-c]tetrazolo[1,5-a]pyridin-7-yl)-2-methylpropan-2-amine (2.00 g, 6.34 mmol) and anhydrous dichloromethane (200 mL). Methanesulfonyl chloride (0.52 mL, 6.66 mmol) was added dropwise. The reaction mixture was allowed to warm to ambient temperature and then was left overnight. Analysis by HPLC indicated that the reaction was only 50% complete. Another equivalent of both triethylamine and methanesulfonyl chloride were added. After 2 hours analysis by HPLC indicated that the reaction was 80% complete. Another equivalent of both triethylamine and methanesulfonyl chloride were added. After 2 hours analysis by HPLC indicated that the reaction was complete. The reaction mixture was diluted with 10% sodium hydroxide (200 mL). The layers were separated and the aqueous layer was extracted with dichloromethane (2×200 mL). The organics were combined, dried over magnesium sulfate, filtered, and concentrated under reduced pressure to provide about 2.5 g of a clear oil. The oil was purified by column chromatography (silica gel eluting with 95/5 dichloromethane/methanol) to provide 1.66 g of N-[2-(8-butyl-5,6-dimethyl-7H-imidazo[4,5-c]tetrazolo[1,5-a]pyridin-7-yl)-1,1-dimethylethyl]methanesulfonamide as a white solid, mp 75.0-90.0° C.

### Example 27

### N-[2-(4-Amino-2-butyl-6,7-dimethyl-1H-imidazo[4,5-c]pyridin-1-yl)-1,1-dimethylethyl]methanesulfonamide

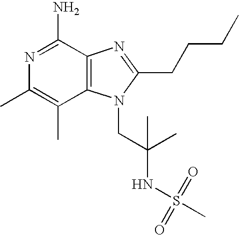

N-[2-(8-Butyl-5,6-dimethyl-7H-imidazo[4,5-c]tetrazolo[1,5-a]pyridin-7-yl)-1,1-dimethylethyl]methanesulfonamide (1.66 g, 4.22 mmol), trifluoroacetic acid (10 mL), and platinum (IV) oxide (0.16 g) were added to a Parr vessel, which was then placed under hydrogen pressure (50 psi, 3.4×10⁵ Pa). For the first six hours, the vessel was flushed with hydrogen every two hours; then it was maintained under hydrogen pressure (50 psi, 3.4×10⁵ Pa) for two days. The reaction mixture was filtered through a layer of CELITE filter aid, and the filter cake was washed with additional trifluoroacetic acid. The filtrate was concentrated under reduced pressure to provide a clear oil. The oil was dissolved in concentrated hydrochloric acid (25 mL) and allowed to stir overnight. The pH of the solution was adjusted to 14 with 10% sodium hydroxide. The resulting mixture was extracted with chloroform (3×200 mL). The organics were combined, dried over magnesium sulfate, filtered, and concentrated under reduced pressure to provide 1.51 g of a white solid. The solid was dissolved in isopropanol (20 mL), combined with 1 M hydrochloric acid in diethyl ether (4.1 mL), and stirred. After 5 minutes a precipitate formed. The mixture was cooled in an ice bath for 1 hour and filtered. The isolated solid was rinsed with diethyl ether to provide 1.2 g of the hydrochloride salt of the desired product. The salt was dissolved in water. The pH of the solution was adjusted to 14 with sodium hydroxide. The resulting mixture was extracted with dichloromethane (3×200 mL). The organics were combined, dried over magnesium sulfate, filtered, and concentrated under reduced pressure to provide about 1 g of a light orange solid. This material was purified by column chromatography (silica gel eluting with 90/10 dichloromethane/methanol) to provide 0.85 g of N-[2-(4-amino-2-butyl-6,7-dimethyl-1H-imidazo[4,5-c]pyridin-1-yl)-1,1-dimethylethyl]methanesulfonamide as a white solid, mp 90.0-92.0° C. Anal. calcd for C₁₇H₂₉N₅O₂S.0.25 H₂O: C, 54.89; H, 7.99; N, 18.83, Found: C, 54.98: H, 7.95; N, 18.46.

### Example 28

### N-[2-(8-Butyl-5,6-dimethyl-7H-imidazo[4,5-c]tetrazolo[1,5-a]pyridin-7-yl)-1,1-dimethylethyl]-N′-cyclohexylurea

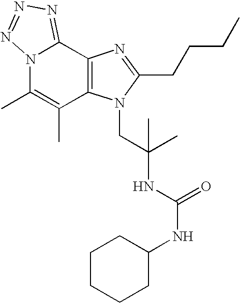

Cyclohexyl isocyanate (4.0 mL, 31.7 mmol) was added dropwise to a chilled (ice bath) mixture of 1-(8-butyl-5,6-dimethyl-7H-imidazo[4,5-c]tetrazolo[1,5-a]pyridin-7-yl)-2-methylpropan-2-amine (2.00 g, 6.34 mmol) and anhydrous dichloromethane (40 mL). The reaction was allowed to warm to ambient temperature and then monitored by HPLC. After 3 days the reaction was complete. The reaction was washed with water. The aqueous was extracted with dichloromethane. The organics were combined, dried over magnesium sulfate, filtered, and concentrated under reduced pressure to provide 2.62 g of N-[2-(8-butyl-5,6-dimethyl-7H-imidazo[4,5-c]tetrazolo[1,5-a]pyridin-7-yl)-1,1-dimethylethyl]-N′-cyclohexylurea as an off white solid, mp 194.0-197.0° C.

### Example 29

### N-[2-(4-Amino-2-butyl-6,7-dimethyl-1H-imidazo[4,5-c]pyridin-1-yl)-1,1-dimethylethyl]-N′-cyclohexylurea

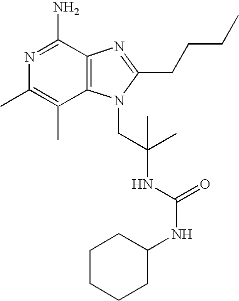

N-[2-(8-Butyl-5,6-dimethyl-7H-imidazo[4,5-c]tetrazolo[1,5-a]pyridin-7-yl)-1,1-dimethylethyl]-N′-cyclohexylurea (2.52 g), trifluoroacetic acid (40 mL), and platinum (IV) oxide (0.25 g) were added to a Parr vessel, which was then placed under hydrogen pressure (50 psi, 3.4×10⁵ Pa). For the first six hours, the vessel was flushed with hydrogen every two hours; then it was maintained under hydrogen pressure (50 psi, 3.4×10⁵ Pa) for two days. The reaction mixture was filtered through a layer of CELITE filter aid, and the filter cake was washed with additional trifluoroacetic acid. The filtrate was concentrated under reduced pressure to provide a light amber oil. The oil was dissolved in concentrated hydrochloric acid (40 mL) and allowed to stir overnight. The pH of the solution was adjusted to 14 with 10% sodium hydroxide. The resulting mixture was extracted with dichloromethane (3×150 mL). The organics were combined, dried over magnesium sulfate, filtered, and concentrated under reduced pressure to provide 2.28 g of a light brown solid. This material was purified by column chromatography (silica gel eluting with 90/10 dichloromethane/methanol) and then recrystallized from isopropanol to provide 1.3 g of a white crystalline solid. The white solid was dissolved in concentrated hydrochloric acid (10 mL) and cooled in an ice bath. Sodium carbonate (7 g) was dissolved in water (20 mL) and then added to the acid solution. A precipitate formed. 50% sodium hydroxide (1 mL) was added to adjust the pH to 14. The solid was isolated by filtration and washed with water. Analysis indicated that this material was a mixture of the free base and the hydrochloride salt. A precipitate formed in the filtrate. This material was isolated by filtration and washed sequentially with 0.5% sodium hydroxide and a very small amount of water to provide 0.25 g of N-[2-(4-amino-2-butyl-6,7-dimethyl-1H-imidazo[4,5-c]pyridin-1-yl)-1,1-dimethylethyl]-N′-cyclohexylurea as a white solid, mp 128.0-130.0° C. Anal. calcd for C₂₃H₃₈N₆O.0.25 H₂O: C, 65.92; H, 9.26; N, 20.05. Found: C, 65.89; H, 9.44; N, 20.07.

### Example 30

### N-[2-(8-Butyl-5,6-dimethyl-7H-imidazo[4,5-c]tetrazolo[1,5-a]pyridin-7-yl)-1,1-dimethylethyl]benzamide

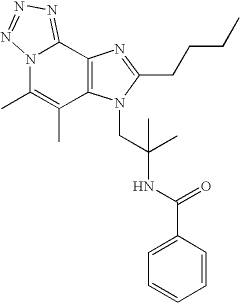

Benzoyl chloride (0.73 mL, 6.32 mmol) was added dropwise to a chilled (ice bath) mixture of 1-(8-butyl-5,6-dimethyl-7H-imidazo[4,5-c]tetrazolo[1,5-a]pyridin-7-yl)-2-methylpropan-2-amine (1.90 g, 6.02 mmol), triethylamine (0.88 mL, 6.32 mmol), and anhydrous dichloromethane (40 mL). The reaction was monitored by thin layer chromatography (silica gel, 90/10 dichloromethane/methanol). When the reaction was complete, the reaction mixture was washed with 5% sodium hydroxide (50 mL). The aqueous was extracted with dichloromethane (2×50 mL). The organics were combined, dried over magnesium sulfate, filtered, and concentrated under reduced pressure to provide about 2.6 g of a light pink solid. This material was purified by column chromatography (silica gel eluting with 95/5 dichloromethane/methanol) to provide 2.40 g of N-[2-(8-butyl-5,6-dimethyl-7H-imidazo[4,5-c]tetrazolo[1,5-a]pyridin-7-yl)-1,1-dimethylethyl]benzamide as a white solid, mp 201.0-204.0° C.

### Example 31

### N-[2-(4-Amino-2-butyl-6,7-dimethyl-1H-imidazo[4,5-c]pyridin-1-yl)-1,1-dimethylethyl]benzamide

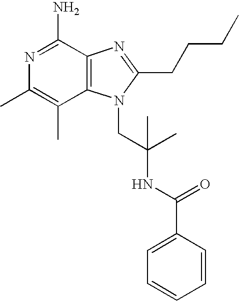

N-[2-(8-Butyl-5,6-dimethyl-7H-imidazo[4,5-c]tetrazolo[1,5-a]pyridin-7-yl)-1,1-dimethylethyl]benzamide (0.50 g, 1.19 mmol), triphenylphosphine (0.47 g, 1.79 mmol), and toluene (10 mL) were combined and heated at reflux until analysis by HPLC indicated that the reaction was complete. The reaction was repeated using 1.89 g of the starting benzamide. The two reaction mixtures were combined and concentrated under reduced pressure. The residue was dissolved in a mixture of methanol (50 mL) and 1 M hydrochloric acid in diethyl ether (12 mL) and heated at reflux for 2 days. The reaction mixture was allowed to cool to ambient temperature and was then concentrated under reduced pressure to provide 5 g of brown oil. The oil was combined with water (100 mL) and heated. The mixture was allowed to cool to ambient temperature and then it was filtered to remove a brown solid (1.8 g) which was discarded. The filtrate was made basic with sodium hydroxide and then extracted with dichloromethane (3×75 mL). The organics were combined, dried over magnesium sulfate, filtered, and concentrated under reduced pressure to provide 2.60 g of light brown solid. The solid was dissolved in isopropanol (25 mL) with heating. The solution was allowed to cool to ambient temperature and then was combined with 1 M hydrochloric acid in diethyl ether (5.70 mL). Diethyl ether (20 mL) was added dropwise and the mixture was cooled in an ice bath to precipitate out the hydrochloride salt. The salt was isolated by filtration and washed sequentially with cold isopropanol and with diethyl ether. The salt was dissolved in water (50 mL) with heating. The solution was allowed to cool to ambient temperature and then it was diluted with a solution of sodium carbonate (2 g) in water (10 mL). The pH was adjusted to 14 with sodium hydroxide. The resulting precipitate was isolated by filtration, washed with water, and then dried under vacuum at 40° C. for 4 hours to provide 1.54 g of N-[2-(4-amino-2-butyl-6,7-dimethyl-1H-imidazo[4,5-c]pyridin-1-yl)-1,1-dimethylethyl]benzamide as a white solid, mp 150.0-152.0° C.. Anal. calcd for C₂₃H₃₁N₅O.0.25 H₂O: C, 69.41; H, 7.98; N, 17.60. Found: C, 69.21; H, 8.10; N, 17.57.

### Example 32

### N-{2-[(5,6-Dimethyl-8-nitrotetrazolo[1,5-a]pyridin-7-yl)amino]-1,1-dimethylethyl}acetamide

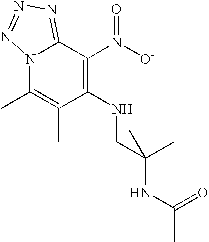 Part A

Acetyl chloride (0.94 mL, 13.2 mmol) was added dropwise to a chilled (ice bath) mixture of N¹-(2-chloro-5,6-dimethyl-3-nitropyridin-4-yl)-2-methylpropane-1,2-diamine (3.0 g, 11.0 mmol), triethylamine (1.8 mL, 13.2 mmol), and dichloromethane (30 mL). After the addition was complete the ice bath was removed. After 1 hour analysis by HPLC indicated that the reaction was complete. The reaction mixture was partitioned between dichloromethane (50 mL) and 5% sodium carbonate. The aqueous was extracted with dichloromethane (2×50 mL). The organics were combined, dried over magnesium sulfate, filtered, and concentrated under reduced pressure to provide about 3.4 g of an orange solid. This material was recrystallized from acetonitrile (15 mL) to provide 2.50 g of N-{2-[(2-chloro-5,6-dimethyl-3-nitropyridin-7-yl)amino]-1,1-dimethylethyl}acetamide as a yellow crystalline solid, mp 93.0-96.0° C. The reaction was scaled up using 50.7 g of N¹-(2-chloro-5,6-dimethyl-3 -nitropyridin-4-yl)-2-methylpropane-1,2-diamine. The crude product was recrystallized from acetonitrile (90 mL) to provide 53.4 g of N-{2-[(2-chloro-5,6-dimethyl-3-nitropyridin-7-yl)amino]-1,1-dimethylethyl}acetamide.

Part B

Using the general method of Example 1 Part C, N-{2-[(2-chloro-5,6-dimethyl-3-nitropyridin-7-yl)amino]-1,1-dimethylethyl}acetamide (53.0 g, 168 mmol) was reacted with sodium azide (21.9 g, 337 mmol) in the presence of cerium (III) chloride heptahydrate (31.4 g, 84 mmol). The crude product was partitioned between dichloromethane (1 L) and water (700 mL). The aqueous was extracted with dichloromethane (2×700 mL). The organics were combined, dried over magnesium sulfate, filtered, and concentrated under reduced pressure to provide about 60 g of a yellow solid. This material was recrystallized from acetonitrile (2 L) to provide 47 g of N-{2-[(5,6-dimethyl-8-nitrotetrazolo[1,5-a]pyridin-7-yl)amino]-1,1-dimethylethyl}acetamide as a yellow crystalline solid, mp 200.0-202.0° C.

### Example 33

### N-{2-[(8-Amino-5,6-dimethyltetrazolo[1,5-a]pyridin-7-yl)amino]-1,1-dimethylethyl}acetamide

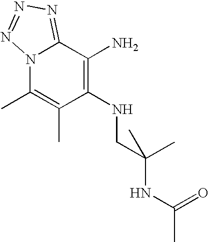

N-{2-[(5,6-Dimethyl-8-nitrotetrazolo[1,5-a]pyridin-7-yl)amino]-1,1-dimethylethyl}acetamide (46.5 g) and acetonitrile (900 mL) were added to a 4 L stainless steel Parr vessel. The starting material did not completely dissolve. The vessel was flushed with nitrogen and 5% platinum on carbon (4.0 g) was added to the mixture. The vessel was placed under hydrogen pressure (50 psi, 3.4×10⁵ Pa) for 16 hours. A white precipitate formed during the reduction. The reaction mixture was flushed with nitrogen for 10 minutes and then diluted with 10% methanol in dichloromethane (500 mL). The reaction mixture was filtered through a layer of CELITE filter aid, and the filter cake was washed sequentially with 10% methanol in dichloromethane (4×500 mL) and methanol (3×250 mL). The filtrate was concentrated under reduced pressure to provide 38.4 g of N-{2-[(8-amino-5,6-dimethyltetrazolo[1,5-a]pyridin-7-yl)amino]-1,1-dimethylethyl}acetamide as a light green solid.

### Example 34

### N-[7-(2-Acetylamino-2-methylpropylamino)-5,6-dimethyltetrazolo[1,5-a]pyridin-8-yl]butyramide

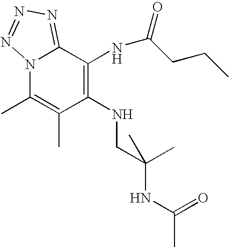

Under a nitrogen atmosphere a solution of butyryl chloride (1.2 mL, 11.7 mmol) in anhydrous dichloromethane (10 mL) was added dropwise over a period of 5 minutes to a chilled (ice bath) mixture of N-{2-[(8-amino-5,6-dimethyltetrazolo[1,5-a]pyridin-7-yl)amino]-1,1-dimethylethyl}acetamide (3.1 g, 10.6 mmol), triethylamine (1.6 mL, 11.7 mmol), and anhydrous dichloromethane (90 mL). The reaction mixture was allowed to warm to ambient temperature and was monitored by TLC (silica gel, 80/20 ethyl acetate/hexanes). After 16 hours additional triethylamine (1 eq) and butyryl chloride (1 eq) were added. Two hours later the reaction was complete. The reaction mixture was partitioned between dichloromethane (250 mL) and 10% sodium hydroxide (250 mL). The aqueous was extracted with dichloromethane (2×250 mL). The organics were combined, dried over magnesium sulfate, filtered, and concentrated under reduced pressure to provide a clear oil. The oil was triturated with toluene (about 100 mL) to provide 2.6 g of N-[7-(2-acetylamino-2-methylpropylamino)-5,6-dimethyltetrazolo[1,5-a]pyridin-8-yl]butryamide as a white solid. The reaction was scaled up using 32.8 g of N-{2-[(8-amino-5,6-dimethyltetrazolo[1,5-a]pyridin-7-yl)amino]-1,1-dimethylethyl}acetamide to provide 36 g of product as a white solid.

### Example 35

### N-[2-(5,6-Dimethyl-8-propyl-7H-imidazo[4,5-c]tetrazolo[1,5-a]pyridin-7-yl)-1,1-dimethylethyl]acetamide

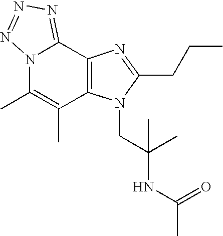

Under a nitrogen atmosphere a mixture of N-[7-(2-acetylamino-2-methylpropylamino)-5,6-dimethyltetrazolo[1,5-a]pyridin-8-yl]butryamide (32.7 g, 90.5 mmol), sodium hydroxide (5.4 g; 136 mmol), ethanol (290 mL), and water (66 mL) was heated at reflux for 17 hours. The reaction mixture was cooled in an ice bath. A white solid (about 20 g) was isolated by filtration. This material was recrystallized from methanol (550 mL) to provide 11.5 g of N-[2-(5,6-dimethyl-8-propyl-7H-imidazo[4,5-c]tetrazolo[1,5-a]pyridin-7-yl)-1,1-dimethylethyl]acetamide as a white crystalline solid, mp 237.0-240.0° C. A second crop (5.0 g) was obtained by reducing the volume of the mother liquor to about 200 mL and then chilling it in a freezer.

### Example 36

### 1-(5,6-Dimethyl-8-propyl-7H-imidazo[4,5-c]tetrazolo[1,5-a]pyridin-7-yl)-2-methylpropan-2-amine

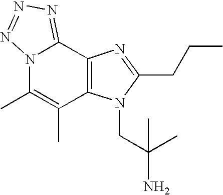

Under a nitrogen atmosphere concentrated hydrochloric acid (144 mL) was slowly added to a mixture of N-[2-(5,6-dimethyl-8-propyl-7H-imidazo[4,5-c]tetrazolo[1,5-a]pyridin-7-yl)-1,1-dimethylethyl]acetamide (16.5 g, 48.0 mmol) and ethanol (220 mL). The reaction mixture was heated at reflux for 15 days. The reaction was allowed to cool to ambient temperature and a precipitate formed. The precipitate was isolated by filtration and then partitioned between dichloromethane (150 mL) and 10% sodium hydroxide (150 mL). The aqueous was extracted with dichloromethane (2×150 mL). The organics were combined, dried over magnesium sulfate, filtered, and concentrated under reduced pressure to provide 8.1 g of 1-(5,6-dimethyl-8-propyl-7H-imidazo[4,5-c]tetrazolo[1,5-a]pyridin-7-yl)-2-methylpropan-2-amine as a white solid, mp 154.0-156.0° C.

### Example 37

### N-[2-(5,6-Dimethyl-8-propyl-7H-imidazo[4,5-c]tetrazolo[1,5-a]pyridin-7-yl)-1,1-dimethylethyl]methanesulfonamide

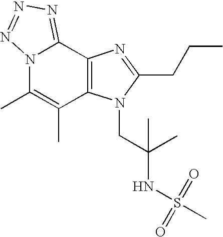

Methanesulfonyl chloride (0.45 mL, 5.81 mmol) was added dropwise to a chilled (ice bath) mixture of 1-(5,6-dimethyl-8-propyl-7H-imidazo[4,5-c]tetrazolo[1,5-a]pyridin-7-yl)-2-methylpropan-2-amine (1.75 g, 5.81 mmol), triethylamine (1.62 mL, 11.6 mmol), and dichloromethane (50 mL). The reaction was allowed to warm to ambient temperature and was monitored by HPLC. After 2 hours additional methanesulfonyl chloride (0.25 eq) was added. Two hours later the reaction was complete and the reaction mixture was concentrated under reduced pressure. The residue was stirred with water and a solid was isolated by filtration. The solid was recrystallized from isopropanol and then purified by column chromatography (silica gel eluting with 80/20 ethyl acetate/methanol) to provide 1.30 g of N-[2-(5,6-dimethyl-8-propyl-7H-imidazo[4,5-c]tetrazolo[1,5-a]pyridin-7-yl)-1,1-dimethylethyl]methanesulfonamide as a white solid, mp 190.0-192.0° C.

### Example 38

### N-[2-(4-Amino-6,7-dimethyl-2-propyl-1H-imidazo[4,5-c]pyridin-1-yl)-1,1-dimethylethyl]methanesulfonamide

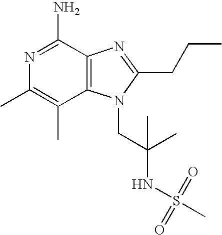

N-[2-(5,6-Dimethyl-8-propyl-7H-imidazo[4,5-c]tetrazolo[1,5-a]pyridin-7-yl)-1,1-dimethylethyl]methanesulfonamide (1.28 g, 3.37 mmol), trifluoroacetic acid (30 mL), and platinum (IV) oxide (0.13 g) were added to a Parr vessel, which was then placed under hydrogen pressure (50 psi, 3.4×10⁵ Pa) for 3 days. The reaction mixture was filtered through a layer of CELITE filter aid, and the filter cake was washed with additional trifluoroacetic acid. The filtrate was concentrated under reduced pressure to provide a light amber oil. The oil was dissolved in concentrated hydrochloric acid (25 mL) and allowed to stir for 2 hours. The pH of the solution was adjusted to 14 with 10% sodium hydroxide. The resulting mixture was extracted with 90/10 dichloromethane/methanol (6×150 mL). The organics were combined, dried over magnesium sulfate, filtered, and concentrated under reduced pressure to provide about 1.15 g of a light pink frothy solid. This material was purified by column chromatography (silica gel eluting with 80/20 chloroform/methanol) to provide 1.05 g of a white frothy solid. The solid was stirred with 10% sodium carbonate (150 mL) at 50° C. for 2 hours and then allowed to cool to ambient temperature. The mixture was extracted as described above to provide 0.80 g of a clear oil. The oil was dissolved in water (100 mL) with heat. The solution was allowed to cool to ambient temperature and then was concentrated under reduced pressure to provide a glassy solid. This material was dried under vacuum at 40° C. overnight to provide 0.80 g of N-[2-(4-amino-6,7-dimethyl-2-propyl-1H-imidazo[4,5-c]pyridin-1-yl)-1,1-dimethylethyl]methanesulfonamide, mp 85.0-93.0° C. Anal. calcd for C₁₆H₂₇N₅O₂S.0.50 H₂O: C, 53.02; H, 7.79; N, 19.32. Found: C, 53.30; H, 7.48; N, 19.30.

### Example 39

### N-[2-(5,6-Dimethyl-8-propyl-7H-imidazo[4,5-c]tetrazolo[1,5-a]pyridin-7-yl)-1,1-dimethylethyl]-N′-cyclohexylurea

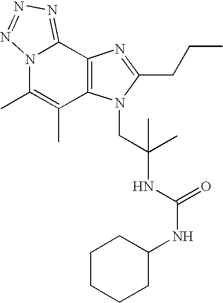

Under a nitrogen atmosphere cyclohexyl isocyanate (1.48 mL, 11.6 mmol) was added dropwise to a chilled (ice bath) mixture of 1-(5,6-dimethyl-8-propyl-7H-imidazo[4,5-c]tetrazolo[1,5-a]pyridin-7-yl)-2-methylpropan-2-amine (1.75 g, 5.81 mmol) and anhydrous dichloromethane (50 mL). The reaction was allowed to warm to ambient temperature and then monitored by HPLC. After 16 hours the reaction was complete. The reaction mixture was concentrated under reduced pressure. The resulting solid was stirred with diethyl ether and then isolated by filtration to provide 2.11 g of N-[2-(5,6-dimethyl-8-propyl-7H-imidazo[4,5-c]tetrazolo[1,5-a]pyridin-7-yl)-1,1-dimethylethyl]-N′-cyclohexylurea as a white solid, mp 196.0-198.0° C.

### Example 40

### N-[2-(4-Amino-6,7-dimethyl-8-propyl-1H-imidazo[4,5-c]pyridin-1-yl)-1,1-dimethylethyl]-N′-cyclohexylurea

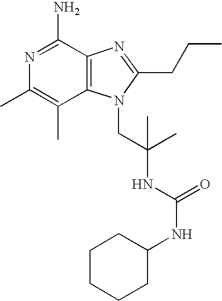

N-[2-(5,6-Dimethyl-8-propyl-7H-imidazo[4,5-c]tetrazolo[1,5-a]pyridin-7-yl)-1,1-dimethylethyl]-N′-cyclohexylurea (2.09 g), trifluoroacetic acid (40 mL), and platinum (IV) oxide (0.21 g) were added to a Parr vessel, which was then placed under hydrogen pressure (50 psi, 3.4×10⁵ Pa) for 2 days. The reaction mixture was filtered through a layer of CELITE filter aid, and the filter cake was washed with additional trifluoroacetic acid. The filtrate was concentrated under reduced pressure to provide a light amber oil. The oil was dissolved in concentrated hydrochloric acid (35 mL) and allowed to stir for 2 hours. The pH of the solution was adjusted to 14 with 10% sodium hydroxide. The resulting mixture was extracted with dichloromethane (3×100 mL). The organics were combined, dried over magnesium sulfate, filtered, and concentrated under reduced pressure to provide about 2.0 g of a light brown solid. The solid was recrystallized from isopropanol (50 mL) to provide 1.5 g of a white crystalline solid. The solid was dissolved in ethanol (50 mL) with heating. The solution was allowed to cool to ambient temperature and then combined with 1 M hydrochloric acid in diethyl ether (4 mL). The solution was allowed to stir for 30 minutes and then it was concentrated under reduced pressure to provide a clear oil. The oil was dissolved in water (100 mL) and the solution was made basic with 50% sodium hydroxide. The resulting precipitate was isolated by filtration and dried. The solid was combined with 10% sodium carbonate (100 mL). The mixture was stirred at 50° C. for 2 hours and then allowed to cool to ambient temperature. A white solid was isolated by filtration and then dried at 50° C. for 2 days to provide 1.09 g of N-[2-(4-amino-6,7-dimethyl-8-propyl-1H-imidazo[4,5-c]pyridin-1-yl)-1,1-dimethylethyl]-N′-cyclohexylurea as a white solid, mp softens at about 125° C. and then melts at 216.0-218.0° C. Anal. calcd for C₂₂H₃₆N₆O.0.75 H₂O: C, 63.81; H, 9.13; N, 20.30. Found: C, 64.00; H, 9.10; N, 20.43

### Example 41

### N-[2-(5,6-Dimethyl-8-propyl-7H-imidazo[4,5-c]tetrazolo[1,5-a]pyridin-7-yl)-1,1-dimethylethyl]benzamide

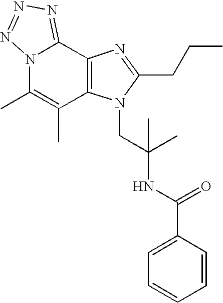

Under a nitrogen atmosphere benzoyl chloride (0.67 mL, 5.81 mmol) was added dropwise to a chilled (ice bath) mixture of 1-(5,6-dimethyl-8-propyl-7H-imidazo[4,5-c]tetrazolo[1,5-a]pyridin-7-yl)-2-methylpropan-2-amine (1.75 g, 5.81 mmol), triethylamine (0.81 mL, 5.81 mmol), and anhydrous dichloromethane (50 mL). The reaction was monitored by HPLC. After 4 hours additional benzoyl chloride (0.25 eq) was added. After an additional 16 hours the reaction mixture was quenched with methanol and then concentrated under reduced pressure to provide a white solid. This material was stirred in water, isolated by filtration, and then dried in a vacuum oven at 50° C. for 16 hours to provide 2.26 g of N-[2-(5,6-dimethyl-8-propyl-7H-imidazo[4,5-c]tetrazolo[1,5-a]pyridin-7-yl)-1,1-dimethylethyl]benzamide as a white solid, mp 221.0-223.0° C.

### Example 42

### N-[2-(4-Amino-6,7-dimethyl-2-propyl-1H-imidazo[4,5-c]pyridin-1-yl)-1,1-dimethylethyl]benzamide

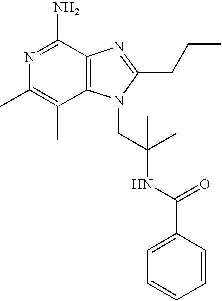

Using the method of Example 31, N-[2-(5,6-dimethyl-8-propyl-7H-imidazo[4,5-c]tetrazolo[1,5-a]pyridin-7-yl)-1,1-dimethylethyl]benzamide (2.20 g) was reacted with triphenylphosphine, hydrolyzed, and then purified to provide 1.16 g of N-[2-(4-amino-6,7-dimethyl-2-propyl-1H-imidazo[4,5-c]pyridin-1-yl)-1,1-dimethylethyl]benzamide as a white solid, mp 208.0-210.0° C. Anal. Calcd for C₂₂H₂₉N₅O: C, 69.63; H, 7.70; N, 18.45. Found: C, 69.60; H, 7.93; N, 18.24.

### Example 43

### N¹-[(5,6-Dimethyl-8-nitrotetrazolo[1,5-a]pyridin-7-yl)-2-methylpropane-1,2-diamine

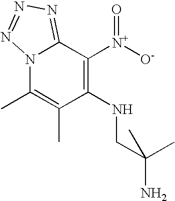

tert-Butyl 2-[(5,6-dimethyl-8-nitrotetrazolo[1,5-a]pyridin-7-yl)amino]-1,1-dimethylethylcarbamate (60.0 g, 158 mmol) was added in portions over a period of 5 minutes to a chilled (ice bath) solution of hydrochloric acid in ethanol (260 mL of 3.6 M). The reaction mixture was allowed to warm to ambient temperature and then was heated at 60° C. for 3 hours. The reaction mixture was allowed to cool to ambient temperature and then it was concentrated under reduced pressure to provide a light yellow solid. The solid was slurried with isopropanol (300 mL), cooled in an ice bath, isolated by filtration, washed sequentially with cold isopropanol and diethyl ether, and then dried under vacuum at 80° C. for 2 hours to provide 53.8 g of N¹-[(5,6-dimethyl-8-nitrotetrazolo[1,5-a]pyridin-7-yl)-2-methylpropane-1,2-diamine, mp 215° C. (decomposes).

### Example 44

### N-Cyclohexyl-N′-{2-[(5,6-dimethyl-8-nitrotetrazolo[1,5-a]pyridin-7-yl)amino-1,1-dimethylethyl}urea

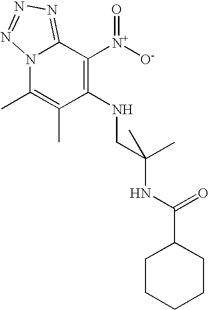

Cyclohexyl isocyanate (22.6 mL, 177 mmol) was added dropwise to a chilled (ice bath) solution of N¹-[(5,6-dimethyl-8-nitrotetrazolo[1,5-a]pyridin-7-yl)-2-methylpropane-1,2-diamine (50.7 g, 161 mmol) and triethylamine (67.1 mL, 482 mmol) in dichloromethane (507 mL). The reaction was allowed to warm to ambient temperature. After 2 hours the reaction mixture was washed with water. The aqueous was extracted with dichloromethane. The organics were combined, dried over magnesium sulfate, filtered, and concentrated under reduced pressure. The residue was stirred with hot acetonitrile (400 mL) and then allowed to cool to ambient temperature. A yellow solid was isolated by filtration and dried to provide 57.3 g of crude product. This material was stirred with isopropanol (400 mL) and then isolated by filtration to provide 51.1 g of N-cyclohexyl-N′-{2-[(5,6-dimethyl-8-nitrotetrazolo[1,5-a]pyridin-7-yl)amino-1,1-dimethylethyl}urea as a yellow solid, mp 180.0-183.0° C.

### Example 45

### N-Cyclohexyl-N′-{2-[(8-amino-5,6-dimethyltetrazolo[1,5-a]pyridin-7-yl)amino]-1,1-dimethylethyl}urea

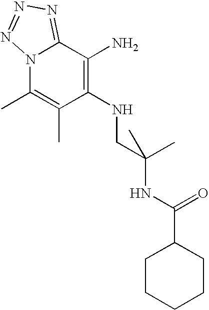

N-Cyclohexyl-N′-{2-[(5,6-dimethyl-8-nitrotetrazolo[1,5-a]pyridin-7-yl)amino]-1,1-dimethylethyl}urea (49.4 g) and acetonitrile (1000 mL) were added to a 2 L Parr vessel. The starting material did not completely dissolve. The vessel was flushed with nitrogen and 5% platinum on carbon (5.0 g) was added to the mixture. The vessel was placed under hydrogen pressure (50 psi, 3.4×10⁵ Pa) for 4 hours. A white precipitate formed during the reduction. The reaction mixture was diluted with dichloromethane (750 mL) and filtered through a layer of CELITE filter aid. The filter cake was washed with 10% methanol in dichloromethane (1000 mL). The filtrate was concentrated under reduced pressure to provide 50.5 g of N-cyclohexyl-N′-{2-[(8-amino-5,6-dimethyltetraazolo[1,5-a]pyridine-7-yl)amino]-1,1-dimethylethyl}urea as a light green frothy solid.

### Example 46

### N-Cyclohexyl-N′-{2-[8-(2-methoxyethyl)-5,6-dimethyl-7H-imidazo[4,5-c]tetrazolo[1,5-a]pyridin-7-yl]-1,1-dimethylethyl}urea

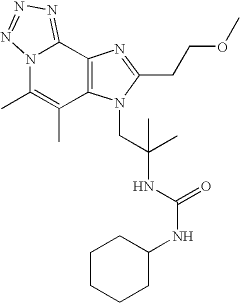 Part A

Hydrochloric acid gas was slowly added to a chilled (ice bath) mixture of 3-methoxypropionitrile (15 mL, 165 mmol), ethanol (9.6 mL of 200 proof, 165 mmol), and anhydrous toluene (100 mL) until the solution was saturated. The reaction mixture was stirred overnight at ambient temperature. Nitrogen was bubbled through the reaction mixture for 10 minutes. The reaction mixture was diluted with diethyl ether (150 mL). The resulting white precipitate was isolated by filtration to provide 20.1 g of ethyl 3-methoxypropionimidate hydrochloride as a white solid.

Part B

Under a nitrogen atmosphere N-cyclohexyl-N′-{2-[(8-amino5,6-dimethyl-tetrazolo[1,5-a]pyridin-7-yl)amino]-1,1-dimethylethyl}urea (1.0 g, 2.67 mmol), ethyl 3-methoxypropionimidate hydrochloride (0.67 g, 4.0 mmol), and 1,2-dichloroethane (10 mL) were heated at 60° C. for 20 hours at which time analysis by HPLC indicated that the reaction was bout 90% complete. The reaction was rerun on a larger scale (×4 for all reagents). When the large scale reaction was complete, the two reaction mixtures were combined and washed with water (100 mL). The aqueous was extracted with dichloromethane (2×100 mL). The organics were combined, dried over sodium sulfate, filtered, and concentrated under reduced pressure to provide a brown oil. The oil was triturated with diethyl ether (150 mL) to provide a brown solid. This material was purified by column chromatography (silica gel eluting with 80/20 ethyl acetate/hexanes) to provide 3.6 g of N-cyclohexyl-N′-{2-[8-(2-methoxyethyl)-5,6-dimethyl-7H-imidazo[4,5-c]tetrazolo[1,5-a]pyridin-7-yl]-1,1-dimethylethyl}urea as a light tan solid.

### Example 47

### N-{2-[4-Amino-2-(2-methoxyethyl)-6,7-dimethyl-1H-imidazo[4,5-c]pyridin-1-yl]-1,1-dimethylethyl}-N′-cyclohexylurea

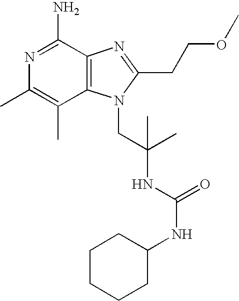

A Parr vessel was charged with N-cylohexyl-N′-{2-[8-(2-methoxyethyl)-5,6-dimethyl-7H-imidazo[4,5-c]tetrazolo[1,5-a]pyridin-7-yl]-1,1-dimethylethyl}urea (3.3 g, 7.46 mmol), concentrated hydrochloric acid (40 mL), and platinum (IV) oxide (0.66 g). The vessel was placed under hydrogen pressure (50 psi, 3.4×10⁵ Pa) for 5 days. The reaction mixture was filtered through a layer of CELITE filter aid. The filter cake was washed with concentrated hydrochloric acid. The pH of the filtrate was adjusted to 12 with saturated sodium carbonate. The mixture was extracted with dichloromethane (3×100 mL). The organics were combined, dried over magnesium sulfate, filtered, and concentrated under reduced pressure to provide about 2.66 g of a white solid. This material was recrystallized from isopropanol (35 mL) to provide 1.55 g of a white crystalline solid. The solid was dissolved in 50/50 water/acetone (100 mL) and then the acetone was allowed to evaporate from the mixture. A precipitate was isolated by filtration and then dried under vacuum at 50° C. overnight to provide 1.06 g of N-{2-[4-amino-2-(2-methoxyethyl)-6,7-dimethyl-1H-imidazo[4,5-c]pyridin-1-yl]-1,1-dimethylethyl}-N′-cyclohexylurea monohydrate as a white crystalline solid, mp 163.0-165.0° C. Anal. calcd for: C₂₂H₃₆N₆O₂.1.00 H₂O: C, 60.80; H, 8.81; N, 19.34. Found: C, 61.13; H, 9.18; N, 19.68.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A process for preparing a 1H-imidazo[4,5-c]pyridin-4-amine compound of the Formula Ia 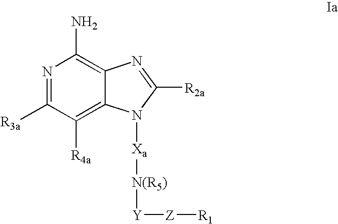
or a pharmaceutically acceptable salt thereof, wherein
Xₐ is alkylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₂ₐ is selected from:
-hydrogen,
-alkyl,
-alkylene-O-alkyl,
-alkylene-S-alkyl, and
-alkyl substituted by one or more substituents selected from:
—OH,
-halogen,
—N(R₆)₂,
—CO—N(R₆)₂,
—CS—N(R₆)₂,
—SO₂—N(R₆)₂,
—NR₆—CO—C₁₋₁₀ alkyl,
—NR₆—CS—C₁₋₁₀ alkyl,
—NR₆—SO₂—C₁₋₁₀ alkyl,
—CO—C₁₋₁₀alkyl,
—CO—O—C₁₋₁₀alkyl,
—N₃,
-heterocyclyl, and
-substituted heterocyclyl; and
R₃ₐ and R₄ₐ are independently selected from hydrogen, alkyl, halogen, alkoxy, amino, alkylamino, dialkylamino, and alkylthio;
R₅ is H or C₁₋₁₀ alkyl, or when R₅ is C₁₋₁₀ alkyl, then R₅ can join with a carbon atom of X to form a ring having the structure 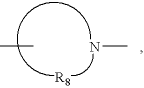
or when R₅ is C₁₋₁₀ alkyl, R₁ is alkyl, and Z is a bond, then R₅ and R₁ can join to form a ring having the structure 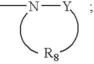
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 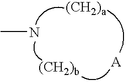
wherein A is selected from —O—, —S(O)₀₋₂—, —N(R₆)—, and —CH₂—; and a and b are independently integers from 1 to 6 with the proviso that a+b is less than or equal to 7;
and
R₈ is C₃₋₈ alkylene;
which process comprises the steps of:
providing a compound of the Formula VIIIa 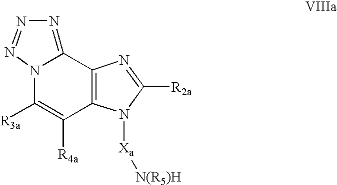
wherein R₂ₐ, R₃ₐ, R₄ₐ, R₅, and Xₐ are as defined above;
reductively removing the tetrazolo ring from the compound of Formula VIIIa to provide a compound of Formula Xa 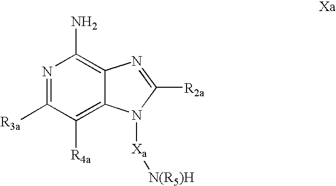
wherein R₂ₐ, R₃ₐ, R₄ₐ, R₅, and Xₐ are as defined above; and
reacting the compound of Formula Xa with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl, wherein R₁, R₇, and R₈ are as defined above; to provide a compound of the Formula Ia or a pharmaceutically acceptable salt thereof.

2. The process of claim 1, further comprising the step of isolating the compound of Formula Ia or a pharmaceutically acceptable salt thereof.

3. The process of claim 1, wherein R₁, R₂ₐ, R₃ₐ, R₄ₐ, R₅, Xₐ, Y and Z are independently selected as follows:
R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇ is C₁₋₄ alkyl which may be interrupted by one or more heteroatoms, R₁ and R₇ can join to form a ring having the structure 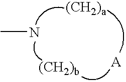
wherein A is selected from —O—, —S(O)₀₋₂—, —N(R₆)—, and —CH₂—; and a and b are independently integers from 1 to 4 with the proviso that a+b is less than or equal to 7;
R₂ₐ is selected from hydrogen, C₁₋₄ alkyl and C₁₋₄ alkylene-O—C₁₋₄ alkyl;
R₃ₐ and R₄ₐ are independently selected from hydrogen and methyl;
R₅ is selected from hydrogen and C₁₋₄ alkyl;
Xa is C₁₋₄ alkylene;
Y is selected from —CO— and —SO₂—; and
Z is selected from a bond and —N(R₇)— wherein R₇ is hydrogen, C₁₋₄ alkyl which may be interrupted by one or more heteroatoms, or R₇ is joined with R₁ as described above.

4. The process of claim 3, wherein
R₁ is selected from methyl, phenyl, and cyclohexyl, or R₁ along with the nitrogen atom to which it is attached is joined with R₇ to form a morpholino ring;
R₂ₐ is selected from hydrogen, methyl, ethyl, propyl, isopropyl, n-butyl, sec-butyl, isobutyl, tert-butyl, 2-methoxyethyl, ethoxymethyl, and cyclopropylmethyl;
R₃ₐ and R₄ₐ are methyl or R₃ₐ is methyl and R₄ₐ is hydrogen;
R₅ is hydrogen;
Xa is selected from ethylene, propylene, and butylene;
Y is selected from —CO— and —SO₂—; and
Z is selected from a bond and —N(R₇)— wherein R₇ is hydrogen, or R₇ along with the nitrogen atom to which it is attached is joined with R₁ to form a morpholino ring.

5. The process of claim 1, further comprising one or more steps selected from steps (i), (ii), (iii), (iv), (v), and (vi) wherein R₂, R₃, R₄, R₅, and X are R₂ₐ, R₃ₐ, R₄ₐ, R₅, and Xₐ, respectively, as defined in claim 1:
(i) providing a compound of the Formula II 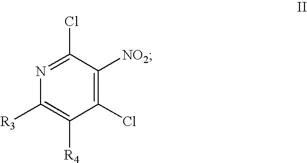
(ii) reacting the compound of Formula II (a) with an amine of the formula N(R₅)—X—NH₂ to provide a compound of Formula XIII 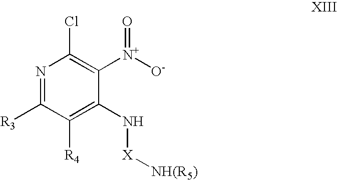
and protecting the —N(R₅)— amino group with a protecting group B, or (b) with an amine of the formula B—N(R₅)—X—NH₂; wherein B is a protecting group for the —N(R₅)— amino group; to provide a compound of the Formula III 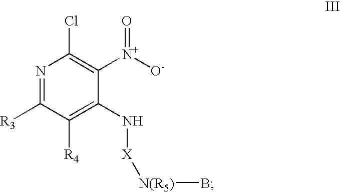
(iii) reacting a compound of the Formula III with an alkali metal azide to provide a compound of Formula IV 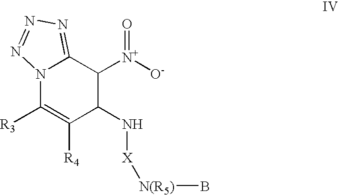
wherein B is as defined above;
(iv) reducing a compound of the Formula IV to provide a compound of Formula V 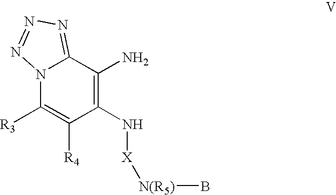
wherein B is as defined above;
(v) reacting a compound of Formula V (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein R₂ is as defined above and each alkyl contains 1 to 8 carbon atoms; to provide a compound of the Formula VII 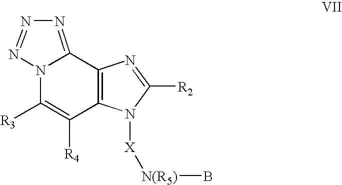
wherein B is as defined above; and
(vi) removing the amine protecting group from a compound of the Formula VII to provide a compound of Formula VIII

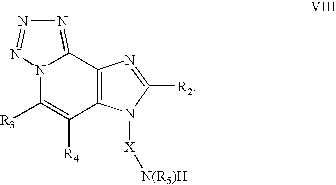

6. The process of claim 5, wherein in step (iii) the compound of Formula III is reacted with the alkali metal azide in the presence of cerium III chloride.

7. The process of claim 5, wherein step (v) includes the steps of
(v-a) reacting a compound of the Formula V with a carboxylic acid of the formula R₂CO₂H or the corresponding acyl halide to form a compound of Formula VI 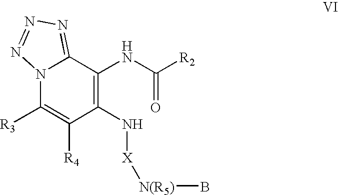
wherein B, R₂, R₃, R₄, R₅, and X are as defined in claim 5; and
(v-b) subjecting the compound of the Formula VI to cyclization conditions, during step (v-a) or subsequent to the completion of step (v-a), to provide a compound of Formula VII 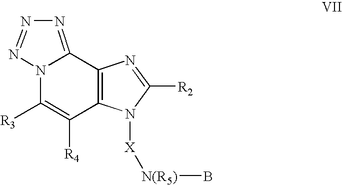
wherein B, R₂, R₃, R₄, R₅, and X are as defined in claim 5.

8. A process for preparing a 1H-imidazo[4,5-c]pyridin-4-amine compound of the Formula I 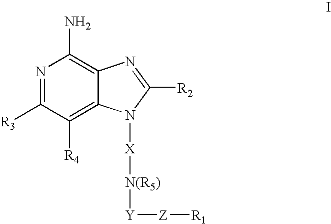
or a pharmaceutically acceptable salt thereof, wherein
X is alkylene or alkenylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl);
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 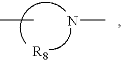
or when R₅ is C₁₋₁₀ alkyl, R₁ is alkyl, and Z is a bond, then R₅ and R₁ can join to form a ring having the structure 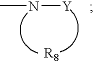
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 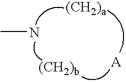
wherein A is selected from —O—, —S(O)₀₋₂—, —N(R₆)—, and —CH₂—; and a and b are independently integers from 1 to 6 with the proviso that a+b is less than or equal to 7;
and
R₈ is C₃₋₈ alkylene;
which process comprises the steps of:
providing a compound of Formula VIII 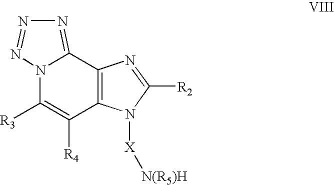
wherein R₂, R₃, R₄, R₅, and X are as defined above;
reacting the compound of the Formula VIII with triphenylphosphine to provide an N-triphenylphosphinyl compound of Formula IX 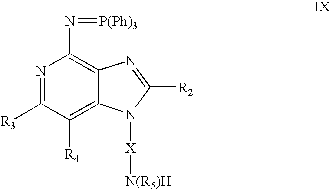
wherein R₂, R₃, R₄, R₅, and X are as defined above;
hydrolyzing the N-triphenylphosphinyl compound of the Formula IX to provide a compound of Formula X 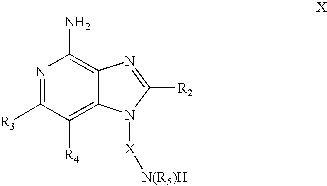
wherein R₂, R₃, R₄, R₅, and X are as defined above; and
reacting the compound of the Formula X with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl; wherein R₁, R₇, and R₈ are as defined above; to provide a compound of the Formula I.

9. The process of claim 8, further comprising the step of isolating the compound of Formula I or a pharmaceutically acceptable salt thereof.

10. The process of claim 8, wherein R₁, R₂, R₃, R₄, R₅, X, Y and Z are independently selected as follows:
R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇ is C₁₋₄ alkyl which may be interrupted by one or more heteroatoms, R₁ and R₇ can join to form a ring having the structure 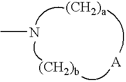
wherein A is selected from —O—, —S(O)₀₋₂—, —N(R₆)—, and —CH₂—; and a and b are independently integers from 1 to 4 with the proviso that a+b is less than or equal to 7;
R₂ is selected from hydrogen, C₁₋₄ alkyl and C₁₋₄ alkylene-O—C₁₋₄ alkyl;
R₃ and R₄ are independently selected from hydrogen and methyl;
R₅ is selected from hydrogen and C₁₋₄ alkyl;
X is selected from C₁₋₄ alkylene and C₁₋₄ alkenylene;
Y is selected from —CO— and —SO₂—; and
Z is selected from a bond and —N(R₇)— wherein R₇ is hydrogen, C₁₋₄ alkyl which may be interrupted by one or more heteroatoms, or R₇ is joined with R₁ as described above.

11. The process of claim 10, wherein
R₁ is selected from methyl, phenyl, and cyclohexyl, or R₁ along with the nitrogen atom to which it is attached is joined with R₇ to form a morpholino ring;
R₂ is selected from hydrogen, methyl, ethyl, propyl, isopropyl, n-butyl, sec-butyl, isobutyl, tert-butyl, 2-methoxyethyl, ethoxymethyl, and cyclopropylmethyl;
R₃ and R₄ are methyl or R₃ is methyl and R₄ is hydrogen;
R₅ is hydrogen;
X is selected from ethylene, propylene, and butylene;
Y is selected from —CO— and —SO₂—; and
Z is selected from a bond and —N(R₇)— wherein R₇ is hydrogen, or R₇ along with the nitrogen atom to which it is attached is joined with R₁ to form a morpholino ring.

12. The process of claim 8, further comprising one or more steps selected from (i), (ii), (iii), (iv), (v), and (vi) wherein R₂, R₃, R₄, R₅, and X are as defined in claim 8:
(i) providing a compound of the Formula II 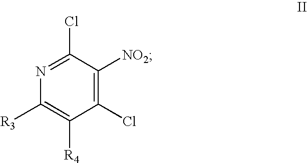
(ii) reacting the compound of Formula II (a) with an amine of the formula N(R₅)—X—NH₂ to provide a compound of Formula XIII 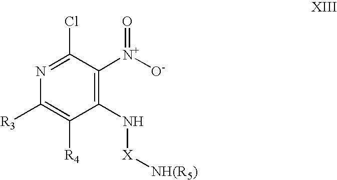
and protecting the —N(R₅)— amino group with a protecting group B, or (b) with an amine of the formula B—N(R₅)—X—NH₂; wherein B is a protecting group for the —N(R₅)— amino group; to provide a compound of the Formula III 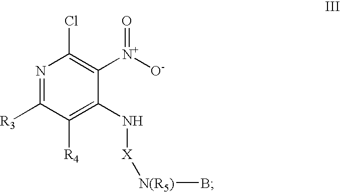
(iii) reacting a compound of the Formula III with an alkali metal azide to provide a compound of Formula IV 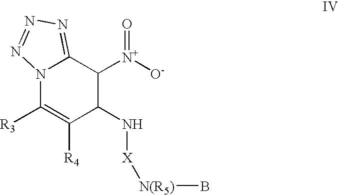
wherein B is as defined above;
(iv) reducing a compound of the Formula IV to provide a compound of Formula V 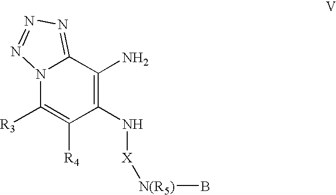
wherein B is as defined above;
(v) reacting a compound of Formula V (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein R₂ is as defined in claim 8, and each alkyl contains 1 to 8 carbon atoms; to provide a compound of Formula VII 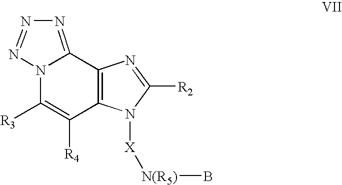
wherein B is as defined above; and
(vi) removing the amine protecting group from a compound of the Formula VII to provide a compound of Formula VIII

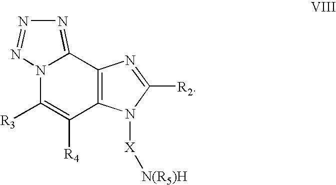

13. The process of claim 12, wherein in step (iii) the compound of Formula III is reacted with the alkali metal azide in the presence of cerium III chloride.

14. The process of claim 12, wherein step (v) includes the steps of
(v-a) reacting a compound of the Formula V with a carboxylic acid of the formula R₂CO₂H or the corresponding acyl halide to form a compound of Formula VI 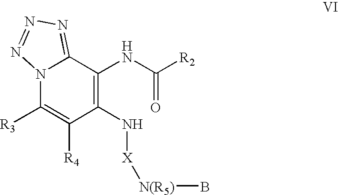
wherein B, R₂, R₃, R₄, R₅, and X are as defined in claim 12; and
(v-b) subjecting the compound of the Formula VI to cyclization conditions, during step (v-a) or subsequent to the completion of step (v-a), to provide a compound of Formula VII 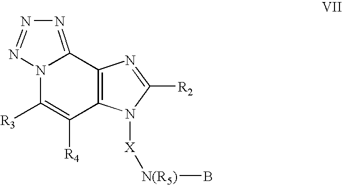
wherein B, R₂, R₃, R₄, R₅, and X are as defined in claim 12.

15. A process for preparing a 1H-imidazo[4,5-c]pyridin-4-amine compound of the Formula Ib 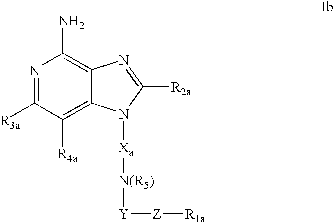
or a pharmaceutically acceptable salt thereof, wherein
Xₐ is alkylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ₐ is alkyl or heterocyclyl, each of which may be unsubstituted or substituted by one or more substituents independently selected from:
-alkyl;
-heterocyclyl;
-substituted cycloalkyl;
-substituted heterocyclyl;
—O-alkyl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl and heterocyclyl, oxo;
R₂ₐ is selected from:
-hydrogen,
-alkyl,
-alkylene-O-alkyl,
-alkylene-S-alkyl, and
-alkyl substituted by one or more substituents selected from:
—OH,
-halogen,
—N(R₆)₂,
—CO—N(R₆)₂,
—CS—N(R₆)₂,
—SO₂—N(R₆)₂,
—NR₆—CO—C₁₋₁₀ alkyl,
—NR₆—CS—C₁₋₁₀ alkyl,
—NR₆—SO₂—C₁₋₁₀ alkyl,
—CO—C₁₋₁₀ alkyl,
—CO—O—C₁₋₁₀ alkyl,
—N₃,
-heterocyclyl, and
-substituted heterocyclyl; and
R₃ₐ and R₄ₐ are independently selected from hydrogen, alkyl, halogen, alkoxy, amino, alkylamino, dialkylamino, and alkylthio;
R₅ is H or C₁₋₁₀ alkyl, or when R₅ is C₁₋₁₀ alkyl, then R₅ can join with a carbon atom of X to form a ring having the structure 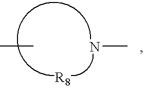
or when R₅ is C₁₋₁₀ alkyl, R₁ is alkyl, and Z is a bond, then R₅ and R₁ can join to form a ring having the structure 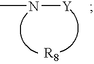
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 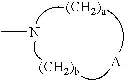
wherein A is selected from —O—, —S(O)₀₋₂—, —N(R₆)—, and —CH₂—; and a and b are independently integers from 1 to 6 with the proviso that a+b is less than or equal to 7;
and
R₈ is C₃₋₈ alkylene;
which process comprises the steps of:
providing a compound of the Formula XIa 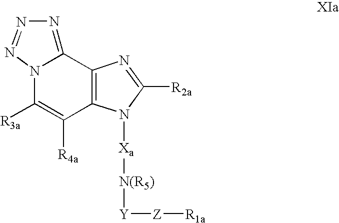
wherein R₁ₐ, R₂ₐ, R₃ₐ, R₄ₐ, R₅, Xₐ, Y, and Z are as defined above; and
reductively removing the tetrazolo ring from the compound of Formula XIa to provide a compound of Formula Ib or a pharmaceutically acceptable salt thereof.

16. The process of claim 15, further comprising the step of isolating the compound of Formula Ib or a pharmaceutically acceptable salt thereof.

17. The process of claim 15, wherein R₁ₐ, R₂ₐ, R₃ₐ, R₄ₐ, R₅, Xₐ, Y and Z are independently selected as follows:
R₁ₐ is selected from C₁₋₄ branched alkyl, C₁₋₄ straight chain alkyl, cycloalkyl, and substituted cycloalkyl, or when R₁ₐ is C₁₋₄ straight chain alkyl, Z is —N(R₇)—, and R₇ is C₁₋₄ alkyl which may be interrupted by one or more heteroatoms, R₁ₐ and R₇ can join to form a ring having the structure 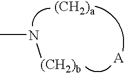
wherein A is selected from —O—, —S(O)₀₋₂—, —N(R₆)—, and —CH₂—; and a and b are independently integers from 1 to 4 with the proviso that a+b is less than or equal to 7;
R₂ₐ is selected from hydrogen, C₁₋₄ alkyl and C₁₋₄ alkylene-O—C₁₋₄ alkyl;
R₃ₐ and R₄ₐ are independently selected from hydrogen and methyl;
R₅ is selected from hydrogen and C₁₋₄ alkyl;
Xa is C₁₋₄ alkylene;
Y is selected from —CO— and —SO₂—; and
Z is selected from a bond and —N(R₇)— wherein R₇ is hydrogen, C₁₋₄ alkyl which may be interrupted by one or more heteroatoms, or R₇ is joined with R₁ₐ as described above.

18. The process of claim 17, wherein
R₁ₐ is selected from methyl, and cyclohexyl, or R₁ₐ along with the nitrogen atom to which it is attached is joined with R₇ to form a morpholino ring;
R₂ₐ is selected from hydrogen, methyl, ethyl, propyl, isopropyl, n-butyl, sec-butyl, isobutyl, tert-butyl, 2-methoxyethyl, ethoxymethyl, and cyclopropylmethyl;
R₃ₐ and R₄ₐ are methyl or R₃ₐ is methyl and R₄ₐ is hydrogen;
R₅ is hydrogen;
Xₐ is selected from ethylene, propylene, and butylene;
Y is selected from —CO— and —SO₂—; and
Z is selected from a bond and —N(R₇)— wherein R₇ is hydrogen or R₇ along with the nitrogen atom to which it is attached is joined with R₁ₐ to form a morpholino ring.

19. The process of claim 15, further comprising the steps of:
providing a compound of the Formula VIIIa 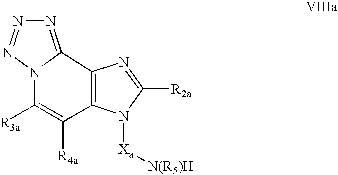
wherein R₂ₐ, R₃ₐ, R₄ₐ, R₅, and Xₐ are as defined in claim 15; and
reacting the compound of Formula VIIIa with a compound selected from R₁ₐ—C(O)Cl, R₁ₐ(R₇)N—C(O)Cl, Cl—R₈—C(O)Cl, R₁ₐ—C(O)OC(O)—R₁ₐ, R₁ₐ(R₇)N—C(O)OC(O)—N(R₇)R₁ₐ, R₁ₐ—N═C═O, R₁ₐ—C(O)—N═C═O, R₁ₐ—S(O)₂—N═C═O, R₁ₐN═C═S, R₁ₐ—C(O)—N═C═S, R₁ₐ—S(O)₂—N═C═S, R₁ₐ—S(O)₂Cl, Cl—R₈—S(O)₂Cl, R₁ₐ—S(O)₂OS(O)₂—R₁ₐ, and R₁ₐ(R₇)N—S(O)₂Cl, wherein R₁ₐ, R₇, and R₈ are as defined in claim 15, to provide a compound of Formula XIa defined in claim 15.

20. The process of claim 19, further comprising one or more steps selected from steps (i), (ii), (iii), (iv), (v), and (vi) wherein R₂, R₃, R₄, R₅, and X are R₂ₐ, R₃ₐ, R₄ₐ, R₅, and Xₐ, respectively, as defined in claim 19:
(i) providing a compound of Formula II 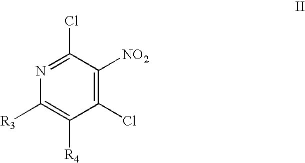
(ii) reacting the compound of Formula II (a) with an amine of the formula N(R₅)—X—NH₂ to provide a compound of Formula XIII 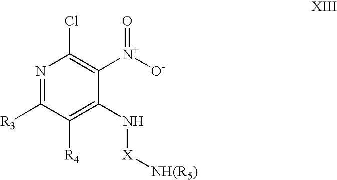
and protecting the —N(R₅)— amino group with a protecting group B, or (b) with an amine of the formula B—N(R₅)—X—NH₂; wherein B is a protecting group for the —N(R₅)— amino group; to provide a compound of the Formula III 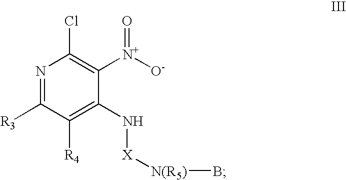
(iii) reacting a compound of the Formula III with an alkali metal azide to provide a compound of Formula IV 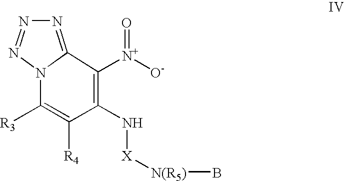
wherein B is as defined above;
(iv) reducing a compound of the Formula IV to provide a compound of Formula V 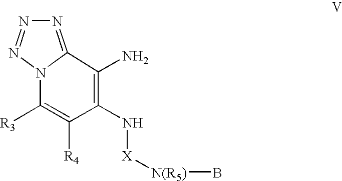
wherein B is as defined above;
(v) reacting a compound of Formula V (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein R₂ is as defined in claim 19 and each alkyl contains 1 to 8 carbon atoms; to provide a compound of the Formula VII 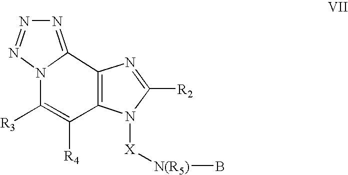
wherein B is as defined above; and
(vi) removing the amine protecting group from a compound of the Formula VII to provide a compound of the Formula VIII

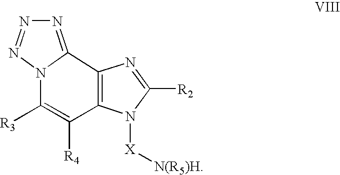

21. The process of claim 20, wherein in step (iii) the compound of Formula III is reacted with the alkali metal azide in the presence of cerium III chloride.

22. The process of claim 20, wherein step (v) includes the steps of
(v-a) reacting a compound of the Formula V with a carboxylic acid of the formula R₂CO₂H or the corresponding acyl halide to form a compound of the Formula VI 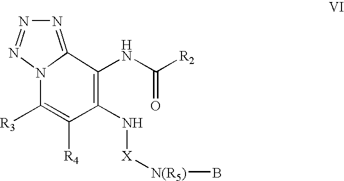
wherein B, R₂, R₃, R₄, R₅, and X are as defined in claim 20; and
(v-b) subjecting the compound of the Formula VI to cyclization conditions, during step (v-a) or subsequent to the completion of step (v-a), to provide a compound of Formula VII 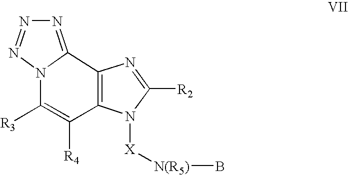
wherein B, R₂, R₃, R₄, R₅, and X are as defined in claim 20.

23. The process of claim 15, further comprising one or more steps selected from steps (i), (ii), (iii), (x), (xi), (xii), and (xiii) wherein R₁, R₂, R₃, R₄, R₅, X, Y, and Z are R₁ₐ, R₂ₐ, R₃ₐ, R₄ₐ, R₅, Xₐ, Y, and Z, respectively, as defined in claim 15:
(i) providing a compound of Formula II 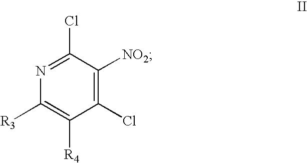
(ii) reacting the compound of Formula II (a) with an amine of the formula N(R₅)—X—NH₂ to provide a compound of Formula XIII 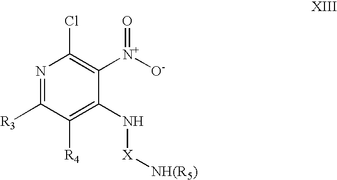
and protecting the —N(R₅)— amino group with a protecting group B, or (b) with an amine of the formula B—N(R₅)—X—NH₂; wherein B is a protecting group for the —N(R₅)— amino group; to provide a compound of the Formula III 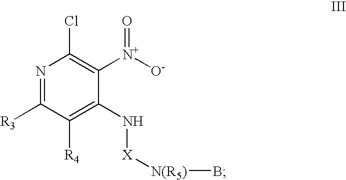
(iii) reacting a compound of the Formula III with an alkali metal azide to provide a compound of Formula IV 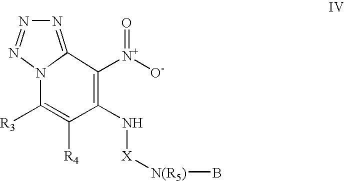
wherein B is as defined above;
(x) removing the amine protecting group from the compound of Formula IV to provide a compound of the Formula XIV 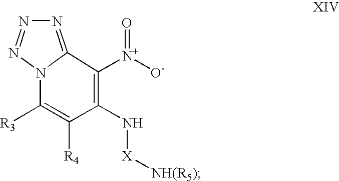
(xi) reacting the compound of Formula XIV with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O,
R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl; wherein R₁, R₇ and R₈ are as defined in claim 15; to provide a compound of Formula XV 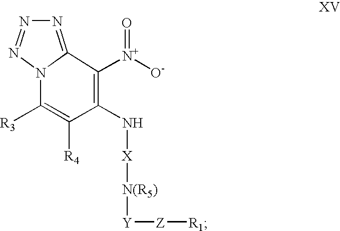
(xii) reducing the compound of Formula XV to provide a compound of the Formula XVI 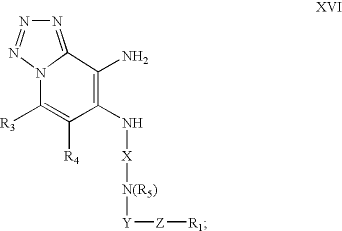
and
(xiii) reacting a compound of Formula XVI (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein each alkyl contains 1 to 8 carbon atoms; to provide a compound of the Formula XI

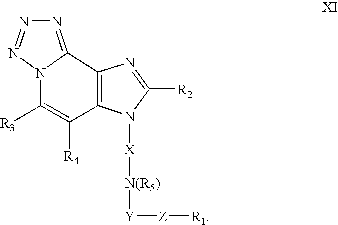

24. The process of claim 23, wherein in step (iii) the compound of Formula III is reacted with the alkali metal azide in the presence of cerium III chloride.

25. A process for preparing a 1H-imidazo[4,5-c]pyridin-4-amine compound of the Formula I 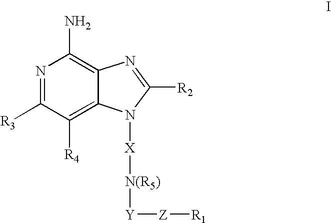
or a pharmaceutically acceptable salt thereof, wherein
X is alkylene or alkenylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl);
R₃ and R₄ are independently selected from hydrogen, alkyl alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 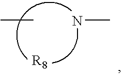
or when R₅ is C₁₋₁₀ alkyl, R₁ is alkyl, and Z is a bond, then R₅ and R₁ can join to form a ring having the structure 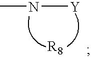
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 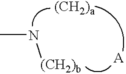
wherein A is selected from —O—, —S(O)₀₋₂—, —N(R₆)—, and —CH₂—; and a and b are independently integers from 1 to 6 with the proviso that a+b is less than or equal to 7;
and
R₈ is C₃₋₈ alkylene;
which process comprises the steps of:
providing a compound of Formula XI 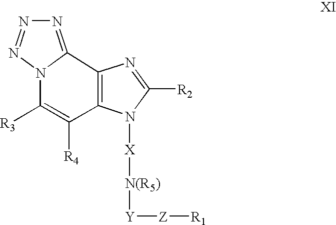
wherein R₁, R₂, R₃, R₄, R₅, X, Y, and Z are as defined above;
reacting the compound of the Formula XI with triphenylphosphine to provide an N-triphenylphosphinyl compound of Formula XII 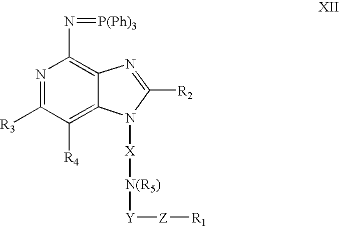
wherein R₁, R₂, R₃, R₄, R₅, X, Y, and Z are as defined above; and
hydrolyzing the N-triphenylphosphinyl compound of the Formula XII to provide a compound of Formula I or a pharmaceutically acceptable salt thereof.

26. The process of claim 25, further comprising the step of isolating the compound of the Formula I or a pharmaceutically acceptable salt thereof.

27. The process of claim 25, wherein R₁, R₂, R₃, R₄, R₅, X, Y and Z are independently selected as follows:
R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇ is C₁₋₄ alkyl which may be interrupted by one or more heteroatoms, R₁ and R₇ can join to form a ring having the structure 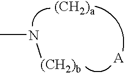
wherein A is selected from —O—, —S(O)₀₋₂—, —N(R₆)—, and —CH₂—; and a and b are independently integers from 1 to 4 with the proviso that a+b is less than or equal to 7;
R₂ is selected from hydrogen, C₁₋₄ alkyl and C₁₋₄ alkylene-O—C₁₋₄ alkyl;
R₃ and R₄ are independently selected from hydrogen and methyl;
R₅ is selected from hydrogen and C₁₋₄ alkyl;
X is selected from C₁₋₄ alkylene and C₁₋₄ alkenylene;
Y is selected from —CO— and —SO₂—; and
Z is selected from a bond and —N(R₇)— wherein R₇ is hydrogen, C₁₋₄ alkyl which may be interrupted by one or more heteroatoms, or R₇ is joined with R₁ as described above.

28. The process of claim 27, wherein
R₁ is selected from methyl, phenyl, and cyclohexyl, or R₁ along with the nitrogen atom to which it is attached is joined with R₇ to form a morpholino ring;
R₂ is selected from hydrogen, methyl, ethyl, propyl, isopropyl, n-butyl, sec-butyl, isobutyl, tert-butyl, 2-methoxyethyl, ethoxymethyl, and cyclopropylmethyl;
R₃ and R₄ are methyl or R₃ is methyl and R₄ is hydrogen;
R₅ is hydrogen;
X is selected from ethylene, propylene, and butylene;
Y is selected from —CO— and —SO₂—; and
Z is selected from a bond and —N(R₇)— wherein R₇ is hydrogen or R₇ along with the nitrogen atom to which it is attached is joined with R₁ to form a morpholino ring.

29. The process of claim 25, further comprising the steps of providing a compound of the Formula VIII 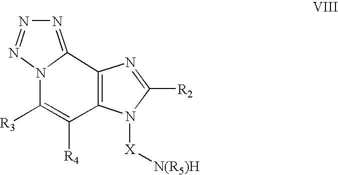
wherein R₂, R₃, R₄, R₅, and X are as defined in claim 25; and
reacting the compound of the Formula VIII with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl, wherein R₁, R₇, and R₈ are as defined in claim 25, to provide a compound of Formula XI defined in claim 25.

30. The process of claim 29, further comprising one or more steps selected from steps (i), (ii), (iii), (iv), (v), and (vi) wherein R₂, R₃, R₄, R₅, and X are as defined in claim 29:
(i) providing a compound of Formula II 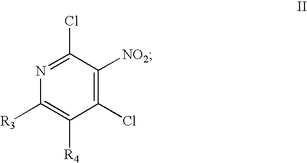
(ii) reacting the compound of Formula II (a) with an amine of the formula N(R₅)—X—NH₂ to provide a compound of Formula XIII 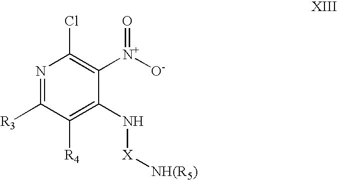
and protecting the —N(R₅)— amino group with a protecting group B, or (b) with an amine of the formula B—N(R₅)—X—NH₂; wherein B is a protecting group for the —N(R₅)— amino group; to provide a compound of the Formula III 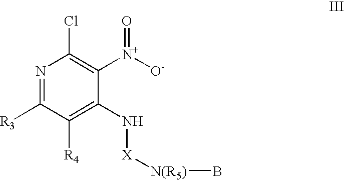
wherein B is as defined above;
(iii) reacting a compound of the Formula III with an alkali metal azide to provide a compound of Formula IV 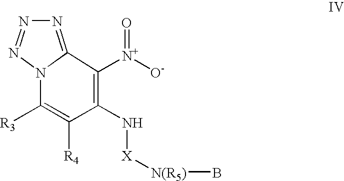
wherein B is as defined above;
(iv) reducing a compound of the Formula IV to provide a compound of Formula V 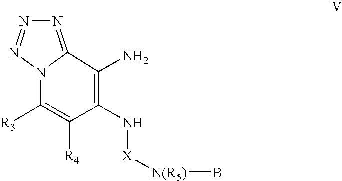
wherein B is as defined above;
(v) reacting a compound of Formula V (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein each alkyl contains 1 to 8 carbon atoms; to provide a compound of Formula VII 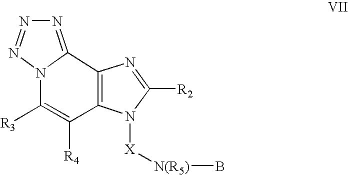
wherein B is as defined above; and
(vi) removing the amine protecting group from a compound of the Formula VII to provide a compound of Formula VIII

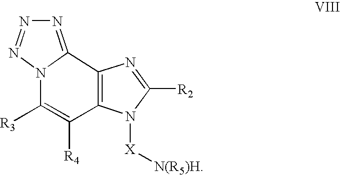

31. The process of claim 30, wherein in step (iii) the compound of Formula III is reacted with the alkali metal azide in the presence of cerium III chloride.

32. The process of claim 30, wherein step (v) includes the steps of (v-a) reacting a compound of the Formula V with a carboxylic acid of the formula R₂CO₂H or the corresponding acyl halide to form a compound of Formula VI 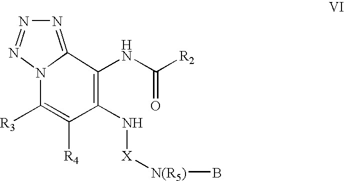
wherein B, R₂, R₃, R₄, R₅, and X are as defined in claim 30; and
(v-b) subjecting the compound of the Formula VI to cyclization conditions, during step (v-a) or subsequent to the completion of step (v-a), to provide a compound of Formula VII 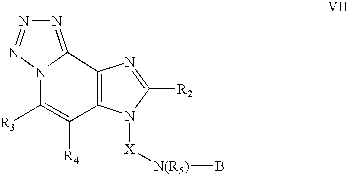
wherein B, R₂, R₃, R₄, R₅, and X are as defined in claim 30.

33. The process of claim 25, further comprising one or more steps selected from steps (i), (ii), (iii), (x), (xi), (xii), and (xiii) wherein R₁, R₂, R₃, R₄, R₅, X, Y, and Z are as defined in claim 25:
(i) providing a compound of Formula II 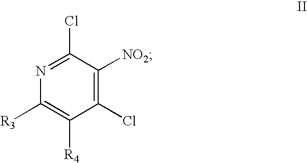
(ii) reacting the compound of Formula II (a) with an amine of the formula N(R₅)—X—NH₂ to provide a compound of Formula XIII 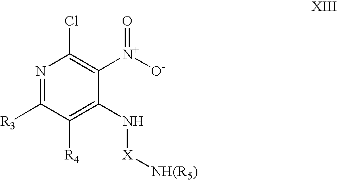
and protecting the —N(R₅)— amino group with a protecting group B, or (b) with an amine of the formula B—N(R₅)—X—NH₂; wherein B is a protecting group for the —N(R₅)— amino group; to provide a compound of the Formula III 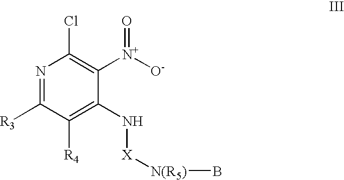
wherein B is as defined above;
(iii) reacting a compound of the Formula III with an alkali metal azide to provide a compound of Formula IV 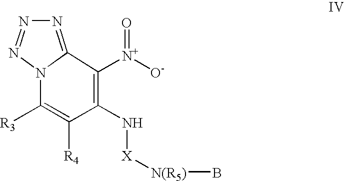
wherein B is as defined above;
(x) removing the amine protecting group from the compound of Formula IV to provide a compound of the Formula XIV 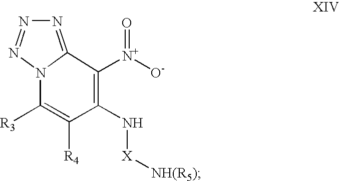
(xi) reacting the compound of Formula XIV with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl, wherein R₁, R₇ and R₈ are as defined in claim 25, to provide a compound of Formula XV 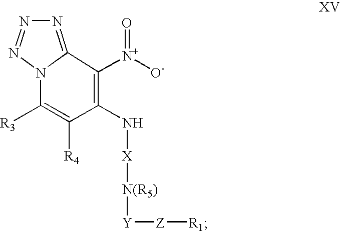
(xii) reducing the compound of Formula XV to provide a compound of the Formula XVI 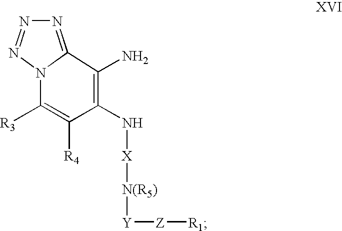
and
(xiii) reacting a compound of Formula XVI (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein each alkyl contains 1 to 8 carbon atoms; to provide a compound of the Formula XI

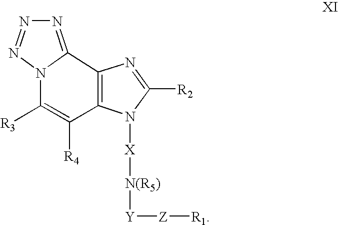

34. The process of claim 33, wherein in step (iii) the compound of Formula III is reacted with the alkali metal azide in the presence of cerium III chloride.

35. A process for preparing a chemical compound comprising the steps of:
providing a compound of Formula III 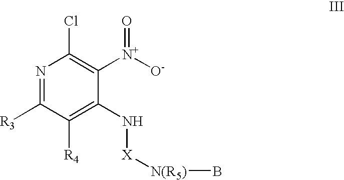
wherein
X is alkylene or alkenylene;
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 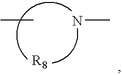
R₈ is C₃₋₈ alkylene; and
B is a protecting group for the —N(R₅)— amino group; and
reacting the compound of the Formula III with an alkali metal azide to provide a compound of Formula IV 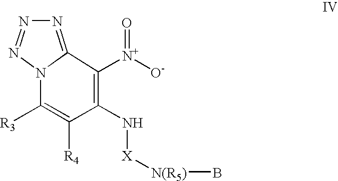
wherein B, R₃, R₄, R₅, and X are as defined above.

36. The process of claim 35, further comprising the step of isolating the compound of Formula IV.

37. The process of claim 35, wherein R₃, R₄, X, and B are independently selected as follows:
R₃ and R₄ are independently selected from hydrogen and methyl;
X is selected from C₁₋₄ alkylene and C₁₋₄ alkenylene; and
B is selected from t-butoxycarbonyl, iso-butoxycarbonyl, benzyloxycarbonyl, ethoxycarbonyl, methoxycarbonyl, benzoyl, pivaloyl, propionyl, acetyl, and phthalimide.

38. The process of claim 37, wherein
R₃ and R₄ are methyl or R₃ is methyl and R₄ is hydrogen;
X is selected from ethylene, propylene, and butylene; and
B is t-butoxycarbonyl.

39. The process of claim 35, wherein the compound of Formula III is reacted with the alkali metal azide in the presence of cerium III chloride.

40. The process of claim 35, further comprising one or more steps selected from steps (iv), (v), (vi),(vii-1), and (viii-a):
(iv) reducing a compound of the Formula IV to provide a compound of the Formula V 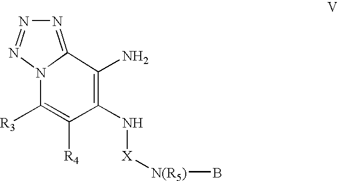
wherein B, R₃, R₄, R₅, and X are as defined in claim 35;
(v) reacting a compound of Formula V (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein each alkyl contains 1 to 8 carbon atoms; R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl); and
each R₆ is independently H or C₁₋₁₀ alkyl; to provide a compound of Formula VII 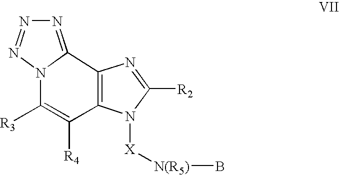
wherein B, R₃, R₄, R₅, and X are as defined in claim 35; and R₂ is as defined above;
(vi) removing the amine protecting group from a compound of the Formula VII to provide a compound of the Formula VIII 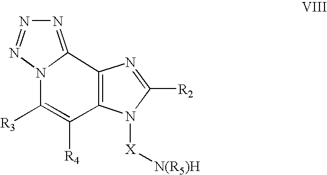
wherein R₂, R₃, R₄, R₅, and X are as defined above in step (v);
(vii-1) providing a compound of the Formula Villa 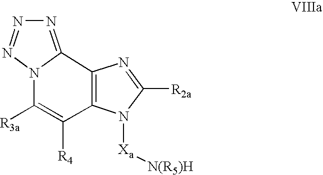
wherein
Xₐ is alkylene;
R₂ₐ is selected from:
-hydrogen,
-alkyl,
-alkylene-O-alkyl,
-alkylene-S-alkyl, and
-alkyl substituted by one or more substituents selected from:
—OH,
-halogen,
—N(R₆)₂,
—CO—N(R₆)₂,
—CS—N(R₆)₂,
—SO₂—N(R₆)₂,
—NR₆—CO—C₁₋₁₀ alkyl,
—NR₆—CS—C₁₋₁₀ alkyl,
—NR₆—SO₂—C₁₋₁₀ alkyl,
—CO—C₁₋₁₀ alkyl,
—CO—O—C₁₋₁₀ alkyl,
—N₃,
-heterocyclyl, and
-substituted heterocyclyl; and
R₃ₐ and R₄ₐ are independently selected from hydrogen, alkyl, halogen, alkoxy, amino, alkylamino, dialkylamino, and alkylthio;
R₅ is H or C₁₋₁₀ alkyl, or when R₅ is C₁₋₁₀ alkyl, then R₅ can join with a carbon atom of Xₐ to form a ring having the structure 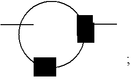
each R₆ is independently H or C₁₋₁₀ alkyl; and
R₈ is C₃₋₈ alkylene;
reductively removing the tetrazolo ring from a compound of the Formula VIIIa to provide a compound of Formula Xa 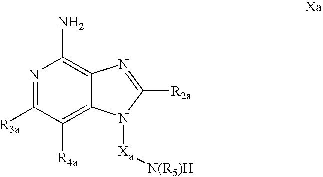
wherein R₂ₐ, R₃ₐ, R₄ₐ, R₅, and Xₐ are as defined above; and
(viii-a) reacting a compound of the Formula Xa with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl;
wherein:
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 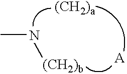
wherein A is selected from —O—, —S(O)₀₋₂—, —N(R₆)—, and —CH₂-; and a and b are independently integers from 1 to 6 with the proviso that a+b is less than or equal to 7;
and
R₈ is C₃₋₈ alkylene; to provide a 1H-imidazo[4,5-c]pyridin-4-amine compound of Formula Ia 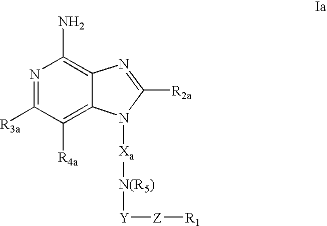
or a pharmaceutically acceptable salt thereof; wherein R₁ is as defined above, R₂ₐ, R₃ₐ, R₄ₐ, and Xₐ are as defined in step (vii-1) above, R₅ is H or C₁₋₁₀ alkyl, or when R₅ is C₁₋₁₀ alkyl, then R₅ can join with a carbon atom of X to form a ring having the structure 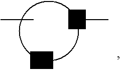
or when R₅ is C₁₋₁₀ alkyl, R₁ is alkyl, and Z is a bond, then R₅ and R₁ can join to form a ring having the structure 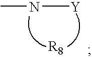
Y is —CO—, —CS—, or —SO₂—; and Z is a bond, —N(R₇)—, —N(R₇)—CO—, or —N(R₇)—SO₂—; with the proviso that when Y is —SO₂— then Z is a bond or —N(R₇)—.

41. The process of claim 40, wherein step (v) includes the steps of
(v-a) reacting a compound of the Formula V with a carboxylic acid of the formula R₂CO₂H or the corresponding acyl halide to form a compound of Formula VI 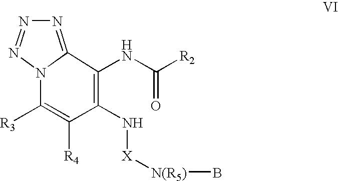
wherein B, R₂, R₃, R₄, R₅, and X are as defined in step (v) of claim 40; and
(v-b) subjecting the compound of the Formula VI to cyclization conditions, during step (v-a) or subsequent to the completion of step (v-a), to provide a compound of Formula VII 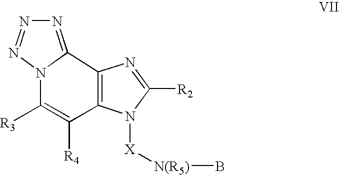
wherein B, R₂, R₃, R₄, R₅, and X are as defined in step (v) of claim 40.

42. The process of claim 40, wherein R₁, R₂, R₃, R₄, R₅, X, R₂ₐ, R₃ₐ, R₄ₐ, Xₐ, Y and Z are independently selected as follows:
R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇ is C₁₋₄ alkyl which may be interrupted by one or more heteroatoms, R₁ and R₇ can join to form a ring having the structure 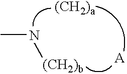
wherein A is selected from —O—, —S(O)₀₋₂—, —N(R₆)—, and —CH₂—; and a and b are independently integers from 1 to 4 with the proviso that a+b is less than or equal to 7;
R₂ and R₂ₐ are selected from hydrogen, C₁₋₄ alkyl and C₁₋₄ alkylene-O—C₁₋₄ alkyl;
R₃, R₃ₐ, R₄, and R₄ₐ are independently selected from hydrogen and methyl;
R₅ is selected from hydrogen and C₁₋₄ alkyl;
X is selected from C₁₋₄ alkylene and C₁₋₄ alkenylene;
Xₐ is C₁₋₄ alkylene;
Y is selected from —CO— and —SO₂—; and
Z is selected from a bond and —N(R₇)— wherein R₇ is hydrogen, C₁₋₄ alkyl which may be interrupted by one or more heteroatoms, or R₇ is joined with R₁ as described above.

43. The process of claim 42, wherein
R₁ is selected from methyl, phenyl, and cyclohexyl, or R₁ along with the nitrogen atom to which it is attached is joined with R₇ to form a morpholino ring;
R₂ and R₂ₐ are selected from hydrogen, methyl, ethyl, propyl, isopropyl, n-butyl, sec-butyl, isobutyl, tert-butyl, 2-methoxyethyl, ethoxymethyl, and cyclopropylmethyl;
R₃, R₃ₐ, R₄, and R₄ₐ are methyl or R₃ and R₃ₐ are methyl and R₄ and R₄ₐ are hydrogen;
R₅ is hydrogen;
X and Xₐ are selected from ethylene, propylene, and butylene;
Y is selected from —CO— and —SO₂—; and
Z is selected from a bond and —N(R₇)— wherein R₇ is hydrogen or R₇ along with the nitrogen atom to which it is attached is joined with R₁ to form a morpholino ring.

44. The process of claim 35, further comprising one or more steps selected from steps (iv), (v), (vi), (vii-a), (vii-b), and (viii-b):
(iv) reducing a compound of the Formula IV to provide a compound of Formula V 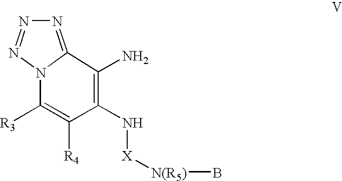
wherein B, R₃, R₄, R₅, and X are as defined in claim 35;
(v) reacting a compound of Formula V (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein each alkyl contains 1 to 8 carbon atoms; R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl); and
each R₆ is independently H or C₁₋₁₀ alkyl;
to provide a compound of Formula VII 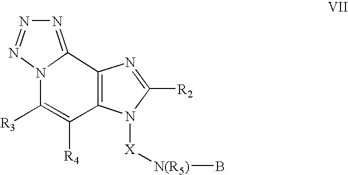
wherein B, R₃, R₄, R₅, and X are as defined in claim 35; and R₂ is as defined above;
(vi) removing the amine protecting group from a compound of the Formula VII to provide a compound of Formula VIII 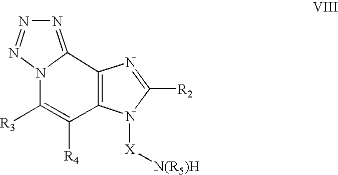
wherein R₂, R₃, R₄, R₅, and X are as defined above in step (v);
(vii-a) reacting a compound of the Formula VIII with triphenylphosphine to provide an N-triphenylphosphinyl compound of Formula IX 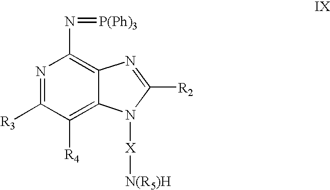
wherein R₂, R₃, R₄, R₅, and X are as defined above in step (v);
(vii-b) hydrolyzing an N-triphenylphosphinyl compound of the Formula IX to provide a compound of Formula X 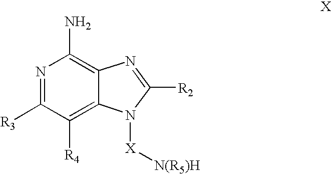
wherein R₂, R₃, R₄, R₅, and X are as defined above in step (v); and
(viii-b) reacting a compound of the Formula X with a compound selected from R₁—C(O)Cl, R₁(R₇)N—C(O)Cl, Cl—R₈—C(O)Cl, R₁—C(O)OC(O)R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl;
wherein:
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 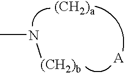
wherein A is selected from —O—, —S(O)₀₋₂—, —N(R₆)—, and —CH₂—; and a and b are independently integers from 1 to 6 with the proviso that a+b is less than or equal to 7;
and
R₈ is C₃₋₈ alkylene;
to provide a 1H-imidazo[4,5-c]pyridin-4-amine compound of Formula I 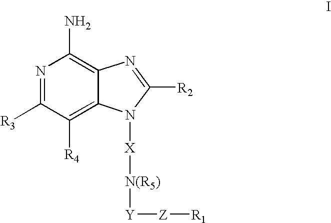
or a pharmaceutically acceptable salt thereof, wherein R₁, R₇, and R₈ are as defined above, R₂, R₃, R₄, and X are as defined in step (v) above, R₅ is H or C₁₋₁₀ alkyl, or when R₅ is C₁₋₁₀ alkyl, then R₅ can join with a carbon atom of X to form a ring having the structure 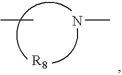
or when R₅ is C₁₋₁₀ alkyl, R₁ is alkyl, and Z is a bond, then R₅ and R₁ can join to form a ring having the structure 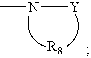
Y is —CO—, —CS—, or —SO₂—; and Z is a bond, —N(R₇)—, —N(R₇)—CO—, or —N(R₇)—SO₂—; with the proviso that when Y is —SO₂— then Z is a bond or —N(R₇)—.

45. The process of claim 44, wherein step (v) includes the steps of
(v-a) reacting a compound of the Formula V with a carboxylic acid of the formula R₂CO₂H or the corresponding acyl halide to form a compound of the Formula VI 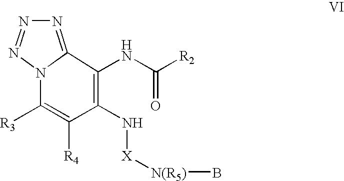
wherein B, R₂, R₃, R₄, R₅, and X are as defined in step (v) of claim 44; and
(v-b) subjecting the compound of the Formula VI to cyclization conditions, during step (v-a) or subsequent to the completion of step (v-a), to provide a compound of Formula VII 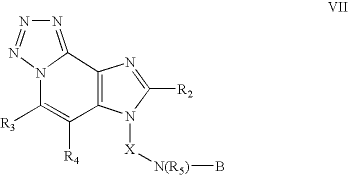
wherein B, R₂, R₃, R₄, R₅, and X are as defined in step (v) of claim 44.

46. The process of claim 44, wherein R₁, R₂, R₃, R₄, R₅, X, Y and Z are independently selected as follows:
R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇ is C₁₋₄ alkyl which may be interrupted by one or more heteroatoms, R₁ and R₇ can join to form a ring having the structure 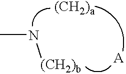
wherein A is selected from —O—, —S(O)₀₋₂—, —N(R₆)—, and —CH₂—; and a and b are independently integers from 1 to 4 with the proviso that a+b is less than or equal to 7;
R₂ is selected from hydrogen, C₁₋₄ alkyl and C₁₋₄ alkylene-O—C₁₋₄ alkyl;
R₃ and R₄ are independently selected from hydrogen and methyl;
R₅ is selected from hydrogen and C₁₋₄ alkyl;
X is selected from C₁₋₄ alkylene and C₁₋₄ alkenylene;
Y is selected from —CO— and —SO₂—; and
Z is selected from a bond and —N(R₇)— wherein R₇ is hydrogen, C₁₋₄ alkyl which may be interrupted by one or more heteroatoms, or R₇ is joined with R₁ as described above.

47. The process of claim 46, wherein
R₁ is selected from methyl, phenyl, and cyclohexyl, or R₁ along with the nitrogen atom to which it is attached is joined with R₇ to form a morpholino ring;
R₂ is selected from hydrogen, methyl, ethyl, propyl, isopropyl, n-butyl, sec-butyl, isobutyl, tert-butyl, 2-methoxyethyl, ethoxymethyl, and cyclopropylmethyl;
R₃ and R₄ are methyl or R₃ is methyl and R₄ is hydrogen;
R₅is hydrogen;
X is selected from ethylene, propylene, and butylene;
Y is selected from —CO— and —SO₂—; and
Z is selected from a bond and —N(R₇)— wherein R₇ is hydrogen or R₇ along with the nitrogen atom to which it is attached is joined with R₁ to form a morpholino ring.

48. The process of claim 35, further comprising one or more steps selected from steps (iv), (v), (vi), (viii-c), and (vii-2):
(iv) reducing a compound of the Formula IV to provide a compound of Formula V 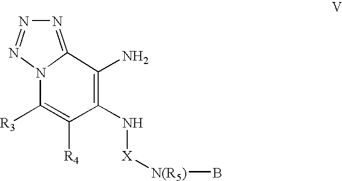
wherein B, R₃, R₄, R₅, and X are as defined in claim 35;
(v) reacting a compound of Formula V (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein each alkyl contains 1 to 8 carbon atoms; R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl), and
each R₆ is independently H or C₁₋₁₀ alkyl;
to provide a compound of Formula VII 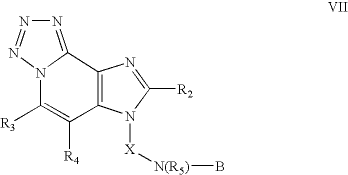
wherein B, R₃, R₄, R₅, and X are as defined in claim 35; and R₂ is as defined above.
(vi) removing the amine protecting group from a compound of the Formula VII to provide a compound of the Formula VIII 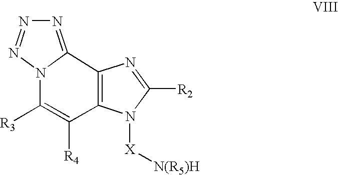
wherein R₂, R₃, R₄, R₅, and X are as defined above in step (v);
(viii-c) providing a compound of the Formula VIIIa 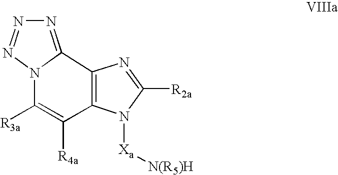
wherein
X₁ is alkylene;
R₂ₐ is selected from:
-hydrogen,
-alkyl,
-alkylene-O-alkyl,
-alkylene-S-alkyl, and
-alkyl substituted by one or more substituents selected from:
—OH,
-halogen,
—N(R₆)₂,
—CO—N(R₆)₂,
—CS—N(R₅)₂,
—SO₂—N(R₆)₂,
—NR₆—CO—C₁₋₁₀ alkyl,
—NR₆—CS—C₁₋₁₀ alkyl,
—NR₆—SO₂—C₁₋₁₀ alkyl,
—CO—C₁₋₁₀ alkyl,
—CO—O—C₁₋₁₀ alkyl,
—N₃,
-heterocyclyl, and
-substituted heterocyclyl; and
R₃ₐ and R₄ₐ are independently selected from hydrogen, alkyl, halogen, alkoxy, amino, alkylamino, dialkylamino, and alkylthio;
R₅ is H or C₁₋₁₀ alkyl, or when R₅ is C₁₋₁₀ alkyl, then R₅ can join with a carbon atom of Xₐ to form a ring having the structure 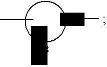
or when R₅ is C₁₋₁₀ alkyl, R₁ is alkyl, and Z is a bond, then R₅ and R₁ can join to form a ring having the structure 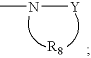
each R₆ is independently H or C₁₋₁₀ alkyl; and
R₈ is C₃₋₈ alkylene;
reacting a compound of the Formula VIIIa with a compound selected from R₁ₐ—C(O)Cl, R₁ₐ(R₇)N—C(O)Cl, Cl—R₈—C(O)Cl, R₁ₐ—C(O)OC(O)—R₁ₐ, R₁ₐ(R₇)N—C(O)OC(O)—N(R₇)R₁ₐ, R₁ₐ—N═C═O, R₁ₐ—C(O)—N═C═O, R₁ₐ—S(O)₂—N═C═O, R₁ₐ—N═C═S, R₁ₐ—C(O)—N═C═S, R₁ₐ—S(O)₂—N═C═S, R₁ₐ—S(O)₂Cl, Cl—R₈—S(O)₂Cl, R₁—S(O)₂OS(O)₂—R₁ₐ, and R₁ₐ(R₇)N—S(O)₂Cl;
wherein:
R₁ₐ is alkyl or heterocyclyl, each of which may be unsubstituted or substituted by one or more substituents independently selected from:
-alkyl;
-heterocyclyl;
-substituted cycloalkyl;
-substituted heterocyclyl;
—O-alkyl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl and heterocyclyl, oxo;
each R₆ is independently H or C₁₋₁₀ alkyl;
R₇ is H or C₁₋₁₀ alkyl which may be interrupted by one or more heteroatoms, or when R₁ₐ is alkyl, Z is —N(R₇)—, and R₇ is C₁₋₁₀ alkyl which may be interrupted by one or more heteroatoms, R₇ and R₁ₐ can join to form a ring having the structure 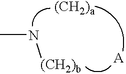
wherein A is selected from —O—, —S(O)₀₋₂—, —N(R₆)—, and —CH₂—; and a and b are independently integers from 1 to 6 with the proviso that a+b is less than or equal to 7; and
R₈ is C₃₋₈ alkylene;
to provide a compound of Formula XIa 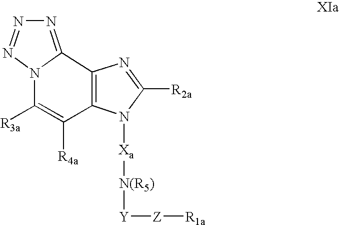
wherein
Xₐ is alkylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—; and
R₁ₐ, R₂ₐ, R₃ₐ, R₄ₐ, and R₅ are as defined above;
(vii-2) reductively removing the tetrazolo ring from the compound of Formula XIa to provide a 1H-imidazo[4,5-c]pyridin-4-amine compound of Formula Ib 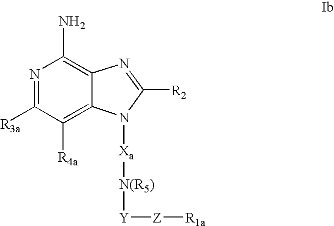
or a pharmaceutically acceptable salt thereof wherein R₁ₐ, R₂ₐ, R₃ₐ, R₄ₐ, R₅, Xₐ, Y, and Z are as defined in Formula XIa.

49. The process of claim 48, wherein step (v) includes the steps of
(v-a) reacting a compound of the Formula V with a carboxylic acid of the formula R₂CO₂H or the corresponding acyl halide to form a compound of Formula VI 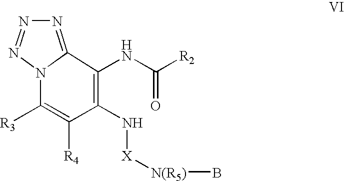
wherein B, R₂, R₃, R₄, R₅, and X are as defined in step (v) of claim 48; and
(v-b) subjecting the compound of the Formula VI to cyclization conditions, during step (v-a) or subsequent to the completion of step (v-a), to provide a compound of Formula VII 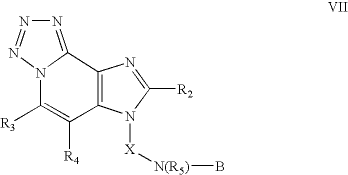
wherein B, R₂, R₃, R₄, R₅, and X are as defined in step (v) claim 48.

50. The process of claim 48, wherein R₁, R₂, R₃,R₄, R₅, X, R₁ₐ, R₂ₐ, R₃ₐ, R₄ₐ, Xₐ, Y and Z are independently selected as follows:
R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇is C₁₋₄ alkyl which may be interrupted by one or more heteroatoms, R₁ and R₇ can join to form a ring having the structure 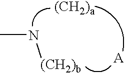
wherein A is selected from —O—, —S(O)₀₋₂—, —N(R₆)—, and —CH₂—; and a and b are independently integers from 1 to 4 with the proviso that a+b is less than or equal to 7;
R₁ₐ is selected from C₁₋₄ branched alkyl, C₁₋₄ straight chain alkyl, cycloalkyl, and substituted cycloalkyl, or when R₁ₐ is C₁₋₄ straight chain alkyl, Z is —N(R₇)—, and R₇ is C₁₋₄ alkyl which may be interrupted by one or more heteroatoms, R₁ₐ and R₇ can join to form a ring having the structure 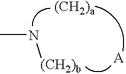
wherein A is selected from —O—, —S(O)₀₋₂—, —N(R₆)—, and —CH₂—; and a and b are independently integers from 1 to 4 with the proviso that a+b is less than or equal to 7;
R₂ and R₂ₐ are selected from hydrogen, C₁₋₄ alkyl and C₁₋₄ alkylene-O—C₁₋₄ alkyl;
R₃, R₃ₐ, R₄, and R₄ₐ are independently selected from hydrogen and methyl;
R₅ is selected from hydrogen and C₁₋₄ alkyl;
X is selected from C₁₋₄ alkylene and C₁₋₄ alkenylene;
X is C₁₋₄ alkylene;
Y is selected from —CO— and —SO₂—; and
Z is selected from a bond and —N(R₇)— wherein R₇ is hydrogen, C₁₋₄ alkyl which may be interrupted by one or more heteroatoms, or R₇ is joined with R₁ or R₁ₐ as described above.

51. The process of claim 50, wherein
R₁ is selected from methyl, phenyl, and cyclohexyl, or R₁ along with the nitrogen atom to which it is attached is joined with R₇ to form a morpholino ring;
R₁ₐ is selected from methyl, and cyclohexyl, or R₁ₐ along with the nitrogen atom to which it is attached is joined with R₇ to form a morpholino ring;
R₂ and R₂ₐ are selected from hydrogen, methyl, ethyl, propyl, isopropyl, n-butyl, sec-butyl, isobutyl, tert-butyl, 2-methoxyethyl, ethoxymethyl, and cyclopropylmethyl;
R₃, R₃ₐ, R₄, and R₄ₐ are methyl or R₃ and R₃ₐ are methyl and R₄ and R₄ₐ are hydrogen;
R₅ is hydrogen;
X and Xₐ are selected from ethylene, propylene, and butylene;
Y is selected from —CO— and —SO₂—; and
Z is selected from a bond and —N(R₇)— wherein R₇ is hydrogen or R₇ along with the nitrogen atom to which it is attached is joined with R₁ or R₁ₐ to form a morpholino ring.

52. The process of claim 35, further comprising one or more steps selected from steps (iv), (v), (vi), (viii-d), (vii-a-1), and (vii-b-1):
(iv) reducing a compound of the Formula IV to provide a compound of the Formula V 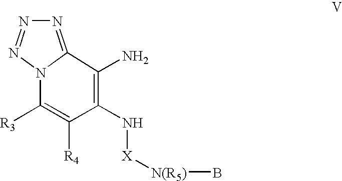
wherein B, R₃, R₄, R₅, and X are as defined in claim 35;
(v) reacting a compound of Formula V (a) with a carboxylic acid of the formula R₂CO₂H; an equivalent thereof selected from the corresponding acyl halide, R₂C(O-alkyl)₃, and R₂C(O-alkyl)₂(O(O═)C-alkyl); or a mixture thereof, or (b) with an imidate of the formula alkyl-O—C(═N)—R₂, wherein each alkyl contains 1 to 8 carbon atoms; R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl), and
each R₆ is independently H or C₁₋₁₀ alkyl;
to provide a compound of Formula VII 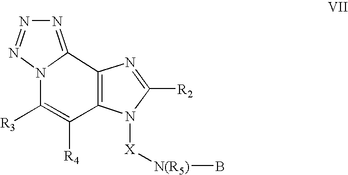
wherein B, R₃, R₄, R₅, and X are as defined in claim 35; and R₂ is as defined above;
(vi) removing the amine protecting group from a compound of the Formula VII to provide a compound of Formula VIII 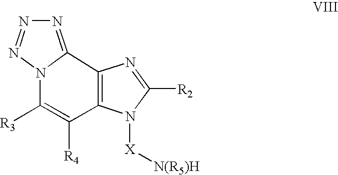
wherein R₂, R₃, R₄, R₅, and X are as defined above in step (v);
(viii-d) reacting a compound of the Formula VIII with a compound selected from R₁—C(O)Cl, R₁(R₇)N—C(O)Cl, Cl—R₈—C(O)Cl, R₁—C(O)OC(O)—R₁, R₁(R₇)N—C(O)OC(O)—N(R₇)R₁, R₁—N═C═O, R₁—C(O)—N═C═O, R₁—S(O)₂—N═C═O, R₁—N═C═S, R₁—C(O)—N═C═S, R₁—S(O)₂—N═C═S, R₁—S(O)₂Cl, Cl—R₈—S(O)₂Cl, R₁—S(O)₂OS(O)₂—R₁, and R₁(R₇)N—S(O)₂Cl;
wherein:
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO—alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 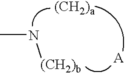
wherein A is selected from —O—, —S(O)₀₋₂—, —N(R₆)—, and —CH₂—; and a and b are independently integers from 1 to 6 with the proviso that a+b is less than or equal to 7;
and
R₈ is C₃₋₈ alkylene;
to provide a compound of Formula XI 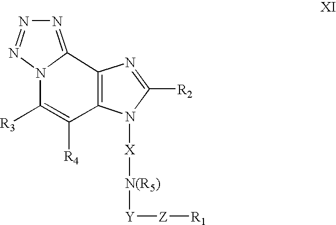
wherein R₂, R₃, R₄, and X are as defined in step (v);
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ is as defined above;
R₅ is H or C₁₋₁₀ alkyl, or when R₅ is C₁₋₁₀ alkyl, then R₅ can join with a carbon atom of X to form a ring having the structure 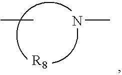
or when R₅ is C₁₋₁₀ alkyl, R₁ is alkyl, and Z is a bond, then R₅ and R₁ can join to form a ring having the structure 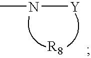
and
R₈ is C₃₋₈ alkylene;
(vii-a-1 ) reacting a compound of the Formula XI with triphenylphosphine to provide an N-triphenylphosphinyl compound of Formula XII 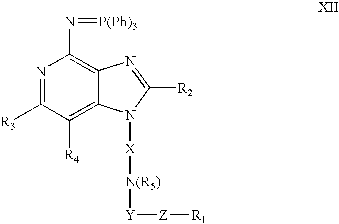
wherein R₁, R₂, R₃, R₄, R₅, X, Y, and Z are as defined in step (viii-d) above; and
(vii-b-1) hydrolyzing an N-triphenylphosphinyl compound of the Formula XII to provide a 1H-imidazo[4,5-c]pyridin-4-amine compound of Formula I 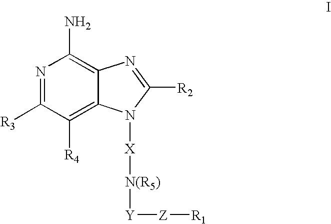
or a pharmaceutically acceptable salt thereof; wherein R₁, R₂, R₃, R₄, R₅, X, Y, and Z are as defined in the Formula XII.

53. The process of claim 52, wherein step (v) includes the steps of
(v-a) reacting a compound of the Formula V with a carboxylic acid of the formula R₂CO₂H or the corresponding acyl halide to form a compound of the Formula VI 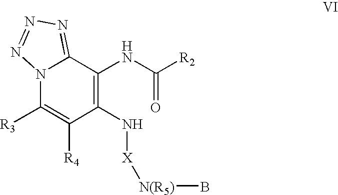
wherein B, R₂, R₃, R₄, R₅, and X are as defined in claim 52; and
(v-b) subjecting the compound of the Formula VI to cyclization conditions, during step (v-a) or subsequent to the completion of step (v-a), to provide a compound of the Formula VII 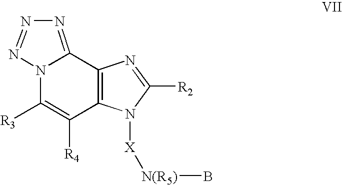
wherein B, R₂, R₃, R₄, R₅, and X are as defined in claim 52.

54. The process of claim 52, wherein R₁, R₂, R₃, R₄, R₅, X, Y and Z are independently selected as follows:
R₁ is selected from C₁₋₄ branched alkyl, C₁₋₄ straight chain alkyl, aryl, substituted aryl, cycloalkyl, and substituted cycloalkyl, or when R₁ is C₁₋₄ straight chain alkyl, Z is —N(R₇)—, and R₇ is C₁₋₄ alkyl which may be interrupted by one or more heteroatoms, R₁ and R₇ can join to form a ring having the structure 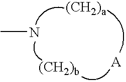
wherein A is selected from —O—, —S(O)₀₋₂—, —N(R₆)—, and —CH₂—; and a and b are independently integers from 1 to 4 with the proviso that a+b is less than or equal to 7;
R₂ is selected from hydrogen, C₁₋₄ alkyl and C₁₋₄ alkylene-O—C₁₋₄ alkyl;
R₃ and R₄ are independently selected from hydrogen and methyl;
R₅ is selected from hydrogen and C₁₋₄ alkyl;
X is selected from C₁₋₄ alkylene and C₁₋₄ alkenylene;
Y is selected from —CO— and —SO₂—; and
Z is selected from a bond and —N(R₇)— wherein R₇ is hydrogen, C₁₋₄ alkyl which may be interrupted by one or more heteroatoms, or R₇ is joined with R₁ as described above.

55. The process of claim 54, wherein
R₁ is selected from methyl, phenyl, and cyclohexyl, or R₁ along with the nitrogen atom to which it is attached is joined with R₇ to form a morpholino ring;
R₂ is selected from hydrogen, methyl, ethyl, propyl, isopropyl, n-butyl, sec-butyl, isobutyl, tert-butyl, 2-methoxyethyl, ethoxymethyl, and cyclopropylmethyl;
R₃ and R₄ are methyl or R₃ is methyl and R₄ is hydrogen;
R₅ is hydrogen;
X is selected from ethylene, propylene, and butylene;
Y is selected from —CO— and —SO₂—; and
Z is selected from a bond and —N(R₇)— wherein R₇ is hydrogen or R₇ along with the nitrogen atom to which it is attached is joined with R₁ to form a morpholino ring.

56. A compound of the Formula XIV 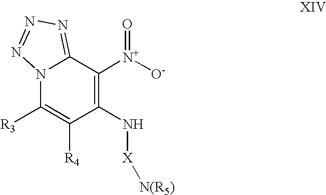
wherein
X is alkylene or alkenylene;
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 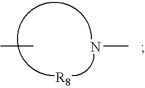
and
R₈ is C₃₋₈ alkylene.

57. A compound of the Formula IV 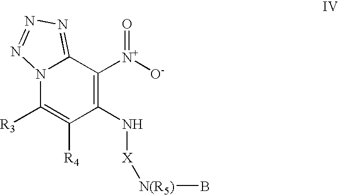
wherein
B is a protecting group for the —N(R₅)— amino group;
X is alkylene or alkenylene;
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 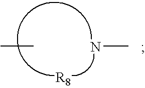
and
R₈ is C₃₋₈ alkylene.

58. A compound of the Formula XV 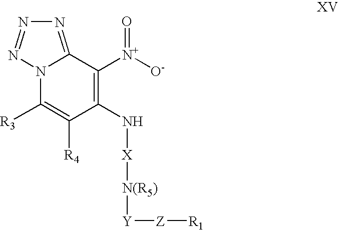
wherein
X is alkylene or alkenylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 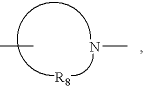
or when R₅ is C₁₋₁₀ alkyl, R₁ is alkyl, and Z is a bond, then R₅ and R₁ can join to form a ring having the structure 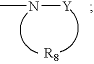
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 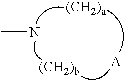
wherein A is selected from —O—, —S(O)₀₋₂—, —N(R)—, and —CH₂—; and a and b are independently integers from 1 to 6 with the proviso that a+b is less than or equal to 7;
and
R₈ is C₃₋₈ alkylene.

59. A compound of the Formula V 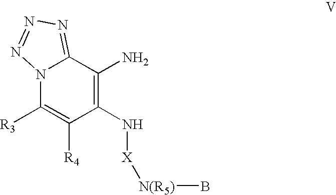
wherein
B is a protecting group for the —N(R₅)— amino group;
X is alkylene or alkenylene;
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 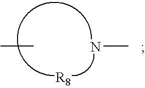
and
R₈ is C₃₋₈ alkylene.

60. A compound of the Formula XVI 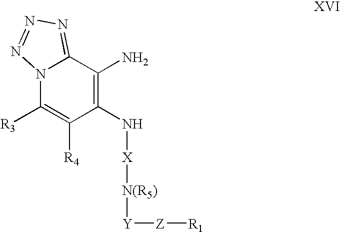
wherein
X is alkylene or alkenylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 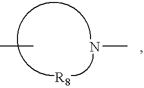
or when R₅ is C₁₋₁₀ alkyl, R₁ is alkyl, and Z is a bond, then R₅ and R₁ can join to form a ring having the structure 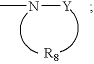
each R₆ is independently H or C₁₋₁₀ alkyl;
R₇ is H or C₁₋₁₀ alkyl which may be interrupted by one or more heteroatoms, or when R₁ is alky, Z is —N(R₇)—, and R₇ is C₁₋₁₀ alkyl which may be interrupted by one or more heteroatoms, R₇ and R₁ can join to form a ring having the structure 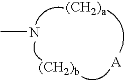
wherein A is selected from —O—, —S(O)₀₋₂—, —N(R₆)—, and —CH₂—; and a and b are independently integers from 1 to 6 with the proviso that a+b is less than or equal to 7;
and
R₈ is C₃₋₈ alkylene.

61. A compound of the Formula VI 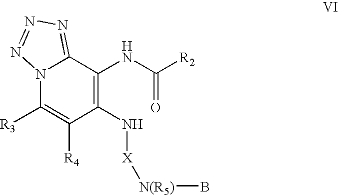
wherein
B is a protecting group for the —N(R₅)— amino group;
X is alkylene or alkenylene;
R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl);
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 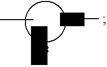
each R₆ is independently H or C₁₋₁₀ alkyl; and
R₈ is C₃₋₈ alkylene.

62. A compound of the Formula VII 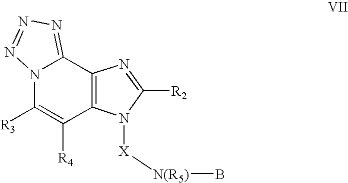
wherein
B is a protecting group for the —N(R₅)— amino group;
X is alkylene or alkenylene;
R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl);
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 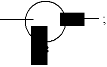
each R₆ is independently H or C₁₋₁₀ alkyl; and
R₈ is C₃₋₈ alkylene.

63. A compound of the Formula VIII 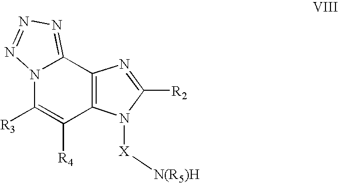
wherein
X is alkylene or alkenylene;
R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl);
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 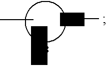
each R₆ is independently H or C₁₋₁₀ alkyl; and
R₈ is C₃₋₈ alkylene.

64. A compound of the Formula XI 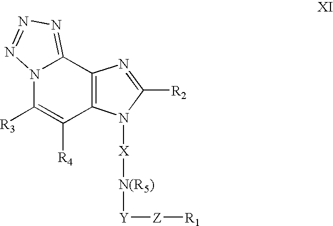
wherein
X is alkylene or alkenylene;
Y is —CO—, —CS—, or —SO₂—;
Z is a bond, —N(R₇)—, —N(R₇)—CO—, or —N(R₇)—SO₂—; with the proviso that when Y is —SO₂— then Z is a bond or —N(R₇)—;
R₁ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkylene)₀₋₁-aryl;
—O-(alkylene)₀₋₁-substituted aryl;
—O-(alkylene)₀₋₁-heteroaryl;
—O-(alkylene)₀₋₁-substituted heteroaryl;
—O-(alkylene)₀₋₁-heterocyclyl;
—O-(alkylene)₀₋₁-substituted heterocyclyl;
—COOH;
—CO—O-alkyl;
—CO-alkyl;
—S(O)₀₋₂-alkyl;
—S(O)₀₋₂-(alkylene)₀₋₁-aryl;
—S(O)₀₋₂-(alkylene)₀₋₁-substituted aryl;
—S(O)₀₋₂-(alkylene)₀₋₁-heteroaryl;
—S(O)₀₋₂-(alkylene)₀₋₁-substituted heteroaryl;
—S(O)₀₋₂-(alkylene)₀₋₁-heterocyclyl;
—S(O)₀₋₂-(alkylene)₀₋₁-substituted heterocyclyl;
-(alkylene)₀₋₁-N(R₆)₂;
-(alkylene)₀₋₁-NR₆—CO—O-alkyl;
-(alkylene)₀₋₁-NR₆—CO-alkyl;
-(alkylene)₀₋₁-NR₆—CO-aryl;
-(alkylene)₀₋₁-NR₆—CO-substituted aryl;
-(alkylene)₀₋₁-NR₆—CO-heteroaryl;
-(alkylene)₀₋₁-NR₆—CO-substituted heteroaryl;
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₂ is selected from:
-hydrogen;
-alkyl;
-alkenyl;
-aryl;
-substituted aryl;
-heteroaryl;
-substituted heteroaryl;
-alkylene-O-alkyl;
-alkylene-S-alkyl;
-alkylene-O-aryl;
-alkylene-S-aryl;
-alkylene-O-alkenyl;
-alkylene-S-alkenyl; and
-alkyl or alkenyl substituted by one or more substituents selected from:
—OH;
-halogen;
—N(R₆)₂;
—CO—N(R₆)₂;
—CS—N(R₆)₂;
—SO₂—N(R₆)₂;
—NR₆—CO—C₁₋₁₀ alkyl;
—NR₆—CS—C₁₋₁₀ alkyl;
—NR₆—SO₂—C₁₋₁₀ alkyl;
—CO—C₁₋₁₀ alkyl;
—CO—O—C₁₋₁₀ alkyl;
—N₃;
-aryl;
-substituted aryl;
-heteroaryl;
-substituted heteroaryl;
-heterocyclyl;
-substituted heterocyclyl;
—CO-aryl;
—CO-(substituted aryl);
—CO-heteroaryl; and
—CO-(substituted heteroaryl);
R₃ and R₄ are independently selected from hydrogen, alkyl, alkenyl, halogen, alkoxy, amino, alkylamino, dialkylamino and alkylthio;
R₅ is H or C₁₋₁₀ alkyl, or when R₅ is C₁₋₁₀ alkyl, then R₅ can join with a carbon atom of X to form a ring having the structure 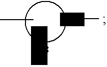
or when R₅ is C₁₋₁₀ alkyl, R₁ is alkyl, and Z is a bond, then R₅ and R₁ can join to form a ring having the structure 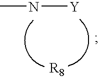
each R₆ is independently H or C₁₋₁₀ alkyl;
R₇ is H or C₁₋₁₀ alkyl which may be interrupted by one or more heteroatoms, or when R₁ is alkyl, Z is —N(R₇)—, and R₇ is C₁₋₁₀ alkyl which may be interrupted by one or more heteroatoms, R₇ and R₁ can join to form a ring having the structure 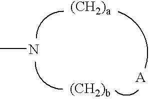
wherein A is selected from —O—, —S(O)₀₋₂—, —N(R₆)—, and —CH₂—; and a and b are independently integers from 1 to 6 with the proviso that a+b is less than or equal to 7;
and R₈ is C₃₋₈ alkylene.
